(19) United States
(12) Patent Application Publication
(10) Pub. No.: US 2011/0105853 A1
(43) Pub. Date: May 5, 2011

(54) SYSTEMS AND METHODS FOR HEALTHCARE DELIVERY, OBSERVATION, AND COMMUNICATION BETWEEN A DE-CENTRALIZED HEALTHCARE SYSTEM AND A PATIENT LIVING AT HOME

(76) Inventors: Richard R. Rakowski, Fairfield, CT (US); David Chess, Stratford, CT (US); Alan London, Toronto (CA); Judy Saita, Franklin, TN (US); Matt Walton, Westport, CT (US)

(21) Appl. No.: 12/879,192

(22) Filed: Sep. 10, 2010

Related U.S. Application Data
(60) Provisional application No. 61/241,719, filed on Sep. 11, 2009.

(51) Int. Cl. A61B 5/00

(52) U.S. Cl. 600/300

(57) ABSTRACT

The disclosure describes de-centralized healthcare systems and methods of using the same to provide healthcare to a subject living at home. A de-centralized healthcare system of the invention provides at or above hospital-level care through the implementation of care pathway protocols for effective treatment of designated health conditions. A subject is monitored by a central station, which is staffed by healthcare practitioners and providers. A dispatch system allows healthcare providers to visit the subject for appointments and deliveries. The de-centralized healthcare system provides for the subject's laboratory, nutrition, and hygiene needs.

This application claims priority to U.S. Provisional Patent Application No. 61/241,719, filed on Sep. 11, 2009, which is incorporated by reference herein in its entirety.

### FIELD OF THE INVENTION

The invention provides systems and methods for the provision of healthcare to subjects outside a hospital environment, for example, at home.

### BACKGROUND

The world is currently suffering a healthcare crisis that is unprecedented in magnitude and touches all people. The surging numbers and climbing age of the global population has created a scarcity of healthcare resources that threatens to erode the quality of healthcare, even in first-world nations where people have access to hospitals and health insurance. Conventional healthcare facilities lack the resources and bed-space necessary to met the increasing demands for quality and outcome-oriented healthcare delivery. Conventional approaches to this issue are not addressing this demand.

### SUMMARY OF THE INVENTION

In some embodiments, the invention contemplates a de-centralized healthcare system comprising: a) a central station staffed by at least one healthcare practitioner; b) a plurality of care pathway protocols, wherein each care pathway protocol comprises a protocol for providing healthcare for a selected designated health condition to a subject in need or want thereof; and c) a communication system for communication between the subject and the healthcare practitioner, wherein the subject resides at a location remote from the central station and is registered with the de-centralized healthcare system.

In some embodiments, the invention contemplates a method of providing healthcare, the method comprising: a) providing a central station for the management of a de-centralized healthcare system, wherein the central station is staffed by at least one healthcare practitioner; b) registering a subject in the de-centralized healthcare system, wherein the subject comprises a designated health condition; c) providing the subject with means for a self-administered healthcare procedure; and d) providing a communication system comprising communication between the subject and the central station, whereby the healthcare practitioner advises the subject on performing the self-administered healthcare procedure according to a care pathway protocol, wherein the subject resides at a location remote from the central station.

In some embodiments, the invention contemplates a method of providing at-home healthcare, the method comprising: a) providing a central station for the management of a de-centralized healthcare system; b) providing a dispatch system staffed by at least one healthcare provider; c) registering a subject in the de-centralized healthcare system, wherein the subject comprises a designated health condition; d) providing a communication system comprising audio/visual communication between the subject and the central station, optionally whereby the subject requests healthcare; e) optionally providing one or more devices and durable medical equipment to the home of the subject, wherein the devices are effective to alert the central station to a need for at-home healthcare; f) dispatching the healthcare provider from the dispatch system to the subject, wherein the subject is in need or want of healthcare; and g) administering a healthcare service to the subject via the healthcare provider according to a care pathway protocol.

In some embodiments, the invention contemplates a method of selecting a subject for a de-centralized healthcare system, the method comprising: a) providing a subject comprising a condition; b) providing a list comprising a plurality of designated health conditions; c) diagnosing the condition of the subject to provide a diagnosed condition; d) comparing the diagnosed condition to the list comprising a plurality of designated health conditions; e) selecting a subject based on the results of step d) to provide a selected subject; and f) registering the selected subject in the de-centralized healthcare system to provide a registered subject, wherein the de-centralized healthcare system comprises: 1) a central station staffed by at least one healthcare practitioner; 2) a plurality of care pathway protocols, wherein each care pathway protocol comprises a protocol for providing healthcare for a selected designated health condition; and 3) a communication system for communication between the registered subject and the healthcare practitioner, wherein the registered subject resides at a location remote from the central station.

In some embodiments, the invention contemplates a method of selecting a subject for a de-centralized healthcare system, the method comprising: a) providing a subject comprising a condition; b) diagnosing the condition of the subject to provide a diagnosed condition; c) performing a cost/benefit analysis for caring for the subject in a hospital system; d) performing a cost/benefit analysis for caring for the subject in the de-centralized healthcare system; e) comparing the results of steps c) and d); f) selecting a subject based on the results of step e) to provide a selected subject; and g) registering the selected subject in the de-centralized healthcare system to provide a registered subject, wherein the de-centralized healthcare system comprises: 1) a central station staffed by at least one healthcare practitioner; 2) a plurality of care pathway protocols, wherein each care pathway protocol comprises a protocol for providing healthcare for a selected designated health condition; and 3) a communication system for communication between the registered subject and the healthcare practitioner, wherein the registered subject resides at a location remote from the central station.

In some embodiments, the invention contemplates a care pathway protocol, comprising:

a) subject qualification and admission, comprising:
1) stability criteria, comprising: subject status, psychological and social safety status; and
2) assessments, comprising: subject's health, home assessments, and nutritional needs;

b) orders, treatments, and activities, comprising: medications, subject activities, nutritional supplementation, procedures, durable medical equipment, and optionally interventions effective to improve the health of the subject;

c) monitoring, comprising: biometric and telecommunication equipment to monitor the subject;

d) education and care coordination/integration, comprising: instructive exercises and literature; and

e) advanced medical directive, comprising: discussions regarding one or both of chronic care management and advanced care planning.

In some embodiments, the invention contemplates a method of providing acute healthcare, the method comprising:

a) qualifying and admitting a subject, comprising:
1) evaluating the subject's stability criteria, comprising the subject's status, and psychological and social safety status; and
2) assessing the subject's health, home, and nutritional needs;

b) issuing orders, treatments, and activities for the subject, comprising: medications, subject activities, nutritional supplementation, procedures, durable medical equipment, and optionally interventions effective to improve the health of the subject;

c) monitoring the subject, comprising: providing biometric and telecommunication equipment to monitor the subject and monitoring the subject with the biometric and telecommunication equipment;

d) educating the subject on care coordination/integration, the educating comprising: instructive exercises and literature; and

e) providing an advanced medical directive, comprising: discussing one or both of chronic care management and advanced care planning

### BRIEF DESCRIPTION OF THE FIGURES

FIG. 1 illustrates an embodiment of the de-centralized healthcare system of the invention disclosed herein. A is a hygiene service. B is a food service. C is a central station comprising: a healthcare practitioner, records, monitoring devices, and communication devices. D is a dispatch system comprising a healthcare provider. E is a laboratory. F is a subject's home comprising: the subject, communication devices, and medical devices. 1) represents a hygiene provider visiting the subject's home to change/clean bed sheets and inspect the sanitation conditions. 2) represents a food service provider visiting the subject's home to provide food, nutritional supplements, vitamins, etc. 3) represents two-way sharing of information. 4) represents two-way sharing of information. 5) represents two-way audio/visual communication. 6) represents two-way sharing of information. 7) represents two-way sharing of information. 8) represents a healthcare provider visiting the subject's home to provide healthcare services; deliver kits; deliver/inspect devices; and make observations. 9) represents a healthcare provider collecting kits; collecting tissue, fluid, and discharge samples; and reporting on subject/device status. 10) represents lab results. 11) represents kits; and tissue, fluid, and discharge samples.

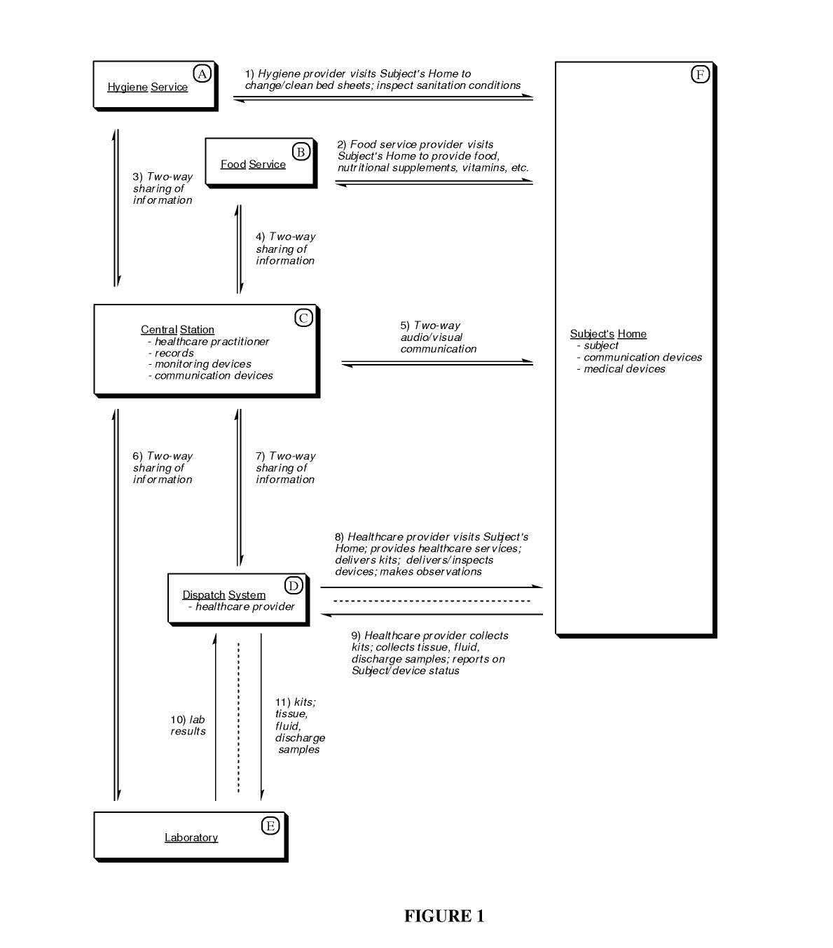

FIG. 2 illustrates an embodiment of the timeline of at-home healthcare as contemplated by the invention. 1) Subject candidate investigation: determine health, symptoms, psychological and social safety status. 2) Subject candidate registration: subject candidate becomes a subject. 3) Subject candidate rejection. 4) Investigate subject's home; equip home with suitable monitoring/communication devices and durable medical equipment. 5) Refer subject candidate to appropriate healthcare facility. 6) Acute phase (about 3-5 days). 7) Post-acute phase (about 0-10 days). 8) Episode prevention phase (ends with physician's approval). 9) Transition subject into long-term care (if appropriate). 10) Subject is discharged; devices and equipment are collected.

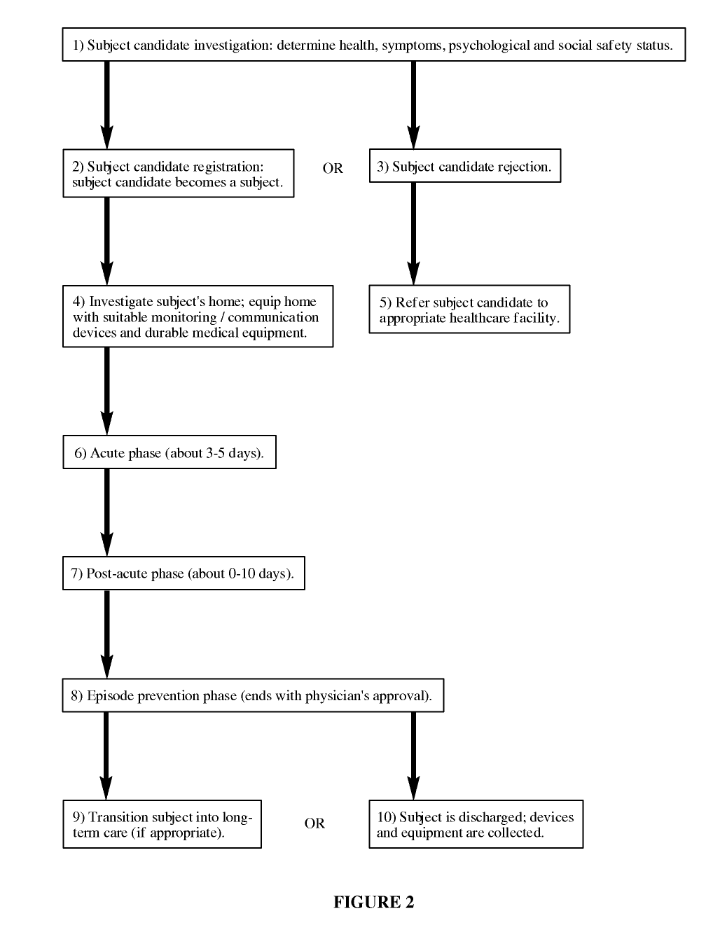

### DETAILED DESCRIPTION OF THE INVENTION

### Definitions

As used herein, the term:

“acute care” means the provision of an urgent medical intervention directed towards a defined diagnosis or condition that would ordinarily result in the temporary admission to a conventional healthcare facility, for example, a hospital. In some embodiments, the condition comprises a designated health condition.

“conventional healthcare facility” means a facility that a subject visits to receive acute care services for a condition. Non-limiting examples of a conventional healthcare facility include a hospital, a clinic, a veterinary hospital, and a hospice.

“de-centralized healthcare facility” means an institution that oversees and provides healthcare services to subjects who do not reside at the facility itself, wherein the healthcare services comprise one or more of a service provided through a communication system and a service provided by a healthcare provider who visits the home of the subject.

“designated health condition” means a condition, indication, handicap, or disease that can be treated equally or more effectively at home under the systems and methods of the instant invention than in a conventional healthcare facility. Herein, this term is not to be confused with the term, “condition,” which is a more general term referring to, for example, the current state of health of a subject or the functional status of a device or instrument. In some embodiments, a designated health condition requires acute care.

“de-centralized healthcare system” or “remote healthcare system,” used interchangeably herein, mean a system that provides healthcare for subjects outside of a conventional healthcare facility, for example, at home. In some embodiments, the de-centralized healthcare system is operated from a de-centralized healthcare facility such as a central station.

“self-administration” means the act of the subject providing healthcare services to oneself. Self-administration is performed at the guidance or direction of the de-centralized healthcare system, and can be done on a scheduled, routine, or emergency basis. A self-administered task can, in some cases, be performed by a relative, friend, or caretaker of the subject who is not associated with the de-centralized healthcare system. The term, “self-administration,” is not intended to describe acts that the subject might choose to perform to oneself without receiving any guidance, direction, or encouragement to do so from the de-centralized healthcare system.

“care pathway protocol” means a collection of methods, instructions, assessments, interventions, education, advanced care planning, care orders, or care coordination that a healthcare practitioner or provider follows when providing healthcare services for a designated health condition. In some embodiments, the methods and instructions comprise proven, evidence-based clinical guidelines and standardized procedures. A specific care pathway protocol corresponds to one or more specific designated health conditions.

“healthcare practitioner” means a healthcare professional comprising all necessary training and licensing for the legal and competent issuance of prescriptions and medical orders. Non-limiting examples of healthcare practitioners comprise doctors of medicine, doctors of osteopathy, and nurse practitioners. A healthcare practitioner falls within the scope of a healthcare provider. In embodiments wherein healthcare treatment is provided to a non-human animal, the healthcare practitioner is a veterinarian.

“healthcare provider” means a healthcare professional who participates in the provision of healthcare to a subject. Non-limiting examples of healthcare providers comprise physicians, clinicians, nurses, pharmacists, paramedics, therapists, and social workers. Non-limiting examples of physicians comprise doctors of medicine, osteopathy, chiropractic, podiatry, optometry, and dentistry. Non-limiting examples of nurses include licensed practical nurses, infusion nurses, and nurse practitioners. A healthcare provider can be a physician's assistant. All healthcare practitioners are healthcare providers, but not all healthcare providers are healthcare practitioners. The healthcare provider is a veterinarian wherein healthcare treatment is provided to a non-human animal, the subject is a non-human animal, or the subject is the owner of an animal.

“healthcare service” means an act provided to a subject with the intent of improving, sustaining, investigating, diagnosing, or monitoring the health of the subject.

“episode” means the period of time during which a subject receives acute care within the home for a designated health condition plus an optional contiguous follow-up period. In some embodiments, the follow-up period comprises at least about 30 days.

“chronic care management” means a period of healthcare provision after the episode and an optional contiguous follow-up period.

“stability criteria” means the physical and social parameters that define the potential for a subject to be cared for in a de-centralized healthcare system.

The concepts of the present invention are not limited to the scope of the embodiments disclosed herein, which are provided for example. The contemplated scope of the invention extends well beyond the scope of the instant disclosure.

Throughout the disclosure, the invention contemplates terms both in the singular and the plural, even if only one of the singular or the plural is used. For example, a reference to a system comprising, “a subject,” also contemplates a system comprising a plurality of subjects.

The invention disclosed herein contemplates systems and methods of providing healthcare to subjects living outside a healthcare facility. The overarching goal of the de-centralized healthcare system described herein is to allow subjects to receive treatment outside of a conventional healthcare facility, such as a hospital or clinic, in some cases in their own homes, and receive healthcare that is equal or superior to the quality of healthcare available in a conventional healthcare facility. In some embodiments of the invention, the healthcare that is provided to the subject comprises acute care. Another aspect of the invention is the provision of healthcare to subjects residing at home, wherein the cost of providing healthcare at the subject's home is lower than the cost of providing analogous healthcare in a conventional healthcare facility, such as a hospital or clinic.

In some embodiments, the subject does not leave home to receive healthcare service from the de-centralized healthcare system. In some embodiments, the subject does not visit the de-centralized healthcare system to receive healthcare service from the de-centralized healthcare system.

The de-centralized healthcare systems disclosed herein uniquely integrate a plurality sites that work together to provide healthcare to the subject. These sites comprise a de-centralized healthcare facility, which provides and oversees the provision of healthcare services; the subject's home, where healthcare services are provided in person or by telecommunication; and conventional healthcare facilities, which provide, for example, staff, medical records, and access to subjects who can enjoy equal or greater quality of life in the system of the instant invention than in the conventional healthcare facility.

A subject's home can be any place where the subject chooses to reside, such as a house, an apartment, a nursing home, a correctional facility, or an assisted living facility.

Careful and diligent determinations have been made as to what varieties of conditions and indications can be managed equally or more effectively at home than in a conventional healthcare facility. These determinations focus primarily on the medical status and social conditions defining the subject's condition, considering the tractability and convenience to the subject of providing such care, and secondarily on fiscal responsibility. To this end, a compilation of designated health conditions has been assimilated. Only subjects comprising at least one designated health condition are considered for registration in the de-centralized healthcare system described herein. A subject comprising at least one designated health condition can comprise other additional health conditions that are not necessarily also designated health conditions, and in this scenario, the systems of the instant invention provide care for the additional health conditions as needed.

In some embodiments, a de-centralized healthcare system of the present invention provides healthcare only to subjects registered with the system.

An additional goal of the inventions disclosed herein is the improvement of the quality of life and happiness of subjects cared for by a de-centralized healthcare system. By spending more time at home, with family, and enjoying personal pursuits, the subjects of the instant invention are happier than subjects being treated at a conventional healthcare facility. This high probability of increasing the quality of life of the subject should encourage subjects to choose both to become registered with a de-centralized healthcare system, and to remain associated with the system for the enjoyment of a higher quality of life.

Quality of life issues are particularly relevant in cases wherein the subject is at or near the end-of-life. End-of-life subjects wish to spend their end-of-life enjoying their remaining time without the constant inconvenience of a conventional healthcare facility lifestyle. The instant invention provides end-of-life subjects with means of increasing the happiness and quality of life, means which are not currently available under other healthcare regimes. For these reasons, the healthcare system of the instant invention would be highly desired by end-of-life subjects.

The qualification and admission of a subject in a de-centralized healthcare system is followed by a preparation process involving two major steps. The first step comprises the outfitting of the subject's home with the facilities, instruments, durable medical equipment, systems, and devices associated with the healthcare system of the instant invention. The second step comprises an at-home education of the subject in the use of the facilities, instruments, systems, and devices associated with the healthcare system of the instant invention. In some embodiments, the subject self-administers a variety of healthcare procedures. The subject becomes knowledgeable of the methods of providing self-administered healthcare services through the education provided by the system, and also through literature provided by the system, and periodic communication with healthcare practitioners and providers. In some embodiments, the healthcare practitioner or provider is a veterinarian, and the subject is the owner of an animal, wherein the animal is the recipient of healthcare services. Systems and methods of the instant invention are effective for monitoring and maintaining the health status of high-value animals.

Systems of the instant invention comprise a variety of healthcare practitioners and providers. These healthcare practitioners and providers can provide healthcare services either remotely or at the subject's home, as described herein.

In some embodiments, the systems and methods comprise communication between the subject and a healthcare practitioner working from within a de-centralized healthcare facility. The healthcare practitioner has access to means and devices effective to monitor the subject from a remote location. By communicating with the healthcare practitioner, the subject can make and conduct appointments for both remote and at-home care delivery, and receive advice, guidance, or instructions on the self-administration of healthcare.

The systems and methods of the instant invention provide for the delivery of healthcare services at the subject's home. A healthcare provider optionally visits the subject for the purpose of providing a variety of healthcare services, as described herein.

An aspect of the instant invention is the development and implementation of a plurality of care pathway protocols. A care pathway protocol comprises one or more of methods, assessments, interventions, education, advanced care planning, care orders, and care coordination for the performance of at least one healthcare service. These methods are based on the opinions and experience of healthcare experts who have thoroughly evaluated the differences between providing healthcare services in the home versus in a conventional healthcare facility and have determined how to accommodate the needs of the subject by means that are practical, accommodating, and convenient to the subject. In some embodiments, a care pathway protocol addresses the methods used to provide healthcare for one or more specific designated health conditions, and is not used to provide healthcare services for any other health condition. All healthcare practitioners and providers are trained extensively in the selection and performance of the appropriate care pathway protocol for a particular designated health condition of the subject at hand.

In the cases of subjects who are unable to provide for their daily personal needs, some embodiments of the invention contemplate providing the subject with hygiene and/or food-delivery services.

The de-centralized healthcare systems described herein provide hospital-level, or above-hospital-level, acute care for subjects residing remotely from the de-centralized healthcare facility. The acute care lasts for an episode, during which the subject resides within the subject's home, and the de-centralized healthcare system provides the acute care. At the end of the episode, the subject is evaluated, and, if appropriate, is optionally discharged from the system. In some embodiments, upon the termination of the episode, an optional follow-up period begins, wherein the system continues to monitor the subject for changes, improvements, regressions, or relapses in the condition of the subject. In some embodiments, the system continues to provide healthcare services for a subject during the follow-up period. In some embodiments, the system provides reduced healthcare services for the subject during the follow-up period. In some embodiments, the follow-up period is effective to reduce the percentage of re-admissions to the system for a subsequent episode of the same designated health condition characteristic of the initial episode. In some embodiments, the follow-up period is terminated, either as scheduled or prematurely, and immediately segues into a subsequent episode. In some embodiments, the follow-up period lasts at least 30 days. During or after the episode, the system may contemplate chronic care management for the subject. If the system admits the subject to chronic care management, then the subject continues to receive healthcare services from the system for a period of time. The period of time may be either designated or undesignated. In some embodiments, the period of time is adjustable. In some embodiments, the staff of the system optionally adjusts the period of time. In some embodiments, the subject's primary care provider optionally adjusts the period of time. In some embodiments, after the episode, the subject is discharged from the de-centralized healthcare system, optionally into the care of the subject's primary care provider. In some embodiments, after the episode the system contemplates advanced care planning for the subject. The advanced care planning for the subject comprises facilitating discussions or enabling access of the subject to information that the subject and optionally the subject's primary care provider use to plan for the subject's healthcare at or near the end-of-life.

A subject candidate for a de-centralized healthcare system can be identified among people living at home in need or want of remote healthcare, or among subjects residing in a conventional healthcare facility, a hospice, or a correctional institution. Subject candidates can also be referred by a conventional healthcare facility, a hospice, a correctional institution, a physician's office, an after hour care center, or a nursing home. Non-limiting examples of candidates include subjects who would otherwise be admitted to a conventional healthcare facility, hospital subjects who are likely to be discharged early, subjects who are at high risk for episode recurrence, and subjects who are suitable for extended (i.e., hospice) care. Prior to transferring a subject from a conventional healthcare facility to at-home care, the subject is observed for a period suitable for a healthcare practitioner to assess the mutual suitability of the subject and the de-centralized healthcare system. This period can be up to an hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 48 hours, several hours, several days, or a week. During this period, the subject can receive preliminary therapy to allow observation of the subject's response to the therapy. The observations are used to determine whether to admit the subject to the de-centralized healthcare system, or re-direct the subject to other suitable healthcare environments.

Information useful in the identification of subject candidates includes electronic and paper medical records, and referral from, for example, a clinical case manager or a healthcare provider or practitioner, such as a primary care provider, a nurse, or a veterinarian. Information can be analyzed by clinical case managers, triage nurses, a subject candidate's primary care provider, or a healthcare provider or practitioner associated with a de-centralized healthcare system disclosed herein.

A healthcare practitioner interviews the subject candidate and reviews any available records. The healthcare practitioner investigates the candidate's history with the designated health condition, including onset, duration, severity, location, changes in condition, symptoms, and treatment. The healthcare practitioner inquires regarding the candidate's history, comprising incidents of other conditions, for example, anemia, angina, anxiety, arrhythmia, benign prostatic hyperplasia, cold agglutinin disease, cancer, cataract, clostridium difficile, chronic heart failure, constipation, chronic obstructive pulmonary disease, cerebrovascular accident, dementia, depression, dyslipidemia, diabetes mellitus, deep vein thrombosis, gastroesophegeal reflux disease, gastrointestinal bleeding, glaucoma, hypertension, hypothyroind, intubation, myocardial infarction, pulmonary embolism, pneumonia, psychiatric history, peptic ulcer disease, peripheral vascular disease, osteoarthritis, obesity, osteoporosis, rheumatoid arthritis, renal insufficiency, seizure, and urinary incontinence. The healthcare practitioner further investigates regarding, for example, surgical history, family availability, mobility, independence, and substance use, for example, alcohol, tobacco, and prescription and non-prescription drugs.

The healthcare practitioner investigates the candidate's symptoms. Non-limiting examples of symptoms to be discussed or treated by the present invention include: a) constitution abnormalities, including, for example, fever, change in mental status, change in function, change in health status, change in weight, and pain; b) gastrointestinal abnormalities, including, for example, nausea, vomiting, obseity, abdominal pain, diarrhea, constipation, melena, heme-occult, dysphagia, dyspepsia, change in appetite, and change in stool; c) neurological abnormalities, including, for example, syncope, aphasia, head ache, vertigo, focal weakness, paraesthesia, seizures, change in speech, change in sensory perceptions, and change in temperature perceptions; d) musculoskeletal abnormalities, including, for example, joint pain, swelling, myalgia, anthralgia, change in range of motion, risk of falls, history of falls, and gait disorder; e) respiratory abnormalities, including, for example, shortness of breath, cough, wheezing, change in sputum amount, change in sputum color, and change in sputum tenacity; f) head-eyes-ears-nose-and-throat (HEENT) abnormalities, including, for example, visual changes, hearing changes, vision aids, tinnitus, dental pain, and dentures; g) genitourinary abnormalities, including, for example, dysuria, hematuria, change in frequency, urgency, nocturia, change in continence, and change in hydration; h) psychiatric abnormalities, including, for example, anxiety, depression, sleep disturbance, combativeness, psychosis, hallucinations, delusions, and substance abuse; i) cardiovascular/pulminaryvascular abnormalities, including, for example, chest pain, palpitations, dizziness, dyspnea on exertion, and edema; and j) dermatological abnormalities, including, for example, rash, pruritus, bruising, and open areas. The healthcare practitioner can optionally observe if the candidate's psychological state appears appropriate, cooperative, anxious, agitated, or depressed. Any of the foregoing symptoms can be monitored by the de-centralized healthcare system. The healthcare practitioner can investigate the candidate's allergies and allergy history.

The healthcare practitioner further orders laboratory work on the subject, such as a blood or urine test, according to laboratory protocols known to one of skill in the art. The healthcare practitioner can further investigate the candidate's nutritional, hygiene, and exercise needs.

Evaluation of the criteria described above leads to a diagnosis and a determination whether the candidate can be registered with the de-centralized healthcare system. A pre-registration safety check is done to ensure that all appropriate treatments, procedures, and investigations have been conducted

A candidate who is suitable for a de-centralized healthcare system is registered with the system, and if previously housed outside the subject's home, is transported to the subject's home. The subject's home is then equipped with devices suitable for provision of at-home healthcare, and the devices are tested for quality control. Follow-up quality control inspections can be made at any time. Devices include monitoring devices and communication devices as described herein, and durable medical equipment. Non-limiting examples of durable medical equipment include beds, adjustable beds, bed wedges, blood pressure units, pulse oximeters, oxygen concentrators, urinals, commodes, wheelchairs, walkers, canes, bedpans, spacers, pneumatic compression stockings, pneumatic compression machines, glucometers, thermometers, pedal cycles, stretch bands, pill boxes, handrails, shower seats, nebulizers, catheters, infusion equipment, IV delivery devices, suction machines, portable oxygen units, and continuous positive airway pressure devices. All devices are catalogued by the central station for identification, maintenance, and re-commissioning for future subjects. The subject is educated on the use of all devices installed in the subject's home, and is optionally provided instructive literature.

The subject can optionally be provided with an assessment regarding the subject's tolerance to exercise, and optionally be provided with an exercise regimen. The subject can optionally be provided with a nutritional/dietary regimen. The subject can optionally be offered access to a chaplain or other emotional support provider.

The de-centralized healthcare system can undergo self-quality control updates as needed to ensure that the most effective and safest healthcare delivery methods are being used in the subject's home. Non-limiting examples of quality control measures include interviews, subject satisfaction questionnaires, failure-point analyses, redundancies, checklists, biofeedback loops, continuing education for subjects and staff, and satisfaction of a subject's primary care provider.

In some embodiments, a de-centralized healthcare facility of the instant invention comprises a central station. The central station, which is operated by a staff comprising at least one healthcare practitioner and optionally comprising support personnel, is a facility that provides healthcare and oversees and administrates the de-centralized healthcare system by managing both the subjects and the healthcare practitioners and providers. In some embodiments, the central station is staffed by a healthcare practitioner. The central station comprises one or more of communication systems; record-keeping systems; computer systems; reference materials, libraries, and systems; a compilation of care pathway protocols; medical or diagnostic kits, equipment, devices, or instruments; prescription and non-prescription drugs; means to write or fill a prescription; and a communication system for contacting a dispatch system. The central station tracks the location, health status, and vital signs of the subjects, and maintains a calendar of events for each subject, for example, appointments, device maintenance, administration of medication, food delivery, hygiene provision, and discharge.

The central station comprises communication systems that are effective for communication with the subject in the subject's home. This communication system allows communication between a healthcare practitioner and a subject. The communication system also enables the healthcare practitioner to monitor and observe the subject through the use of facilities, systems, and devices located within the subject's home for the purpose of assessing the subject's health status. Based on the healthcare practitioner's observations, the healthcare practitioner can initiate correspondence with the subject, by written, audio, visual, or audio/visual communication. In some embodiments, the correspondence is initiated by an emergency device within the subject's home, which alerts the healthcare practitioner at the central station to the need or want of correspondence or healthcare on the part of the subject. In some embodiments, the healthcare practitioner communicates with a relative, friend, or caretaker of the subject in the subject's home. In some embodiments, the healthcare practitioner communicates with a healthcare provider, employed by the de-centralized healthcare system, in the subject's home.

The central station maintains record-keeping systems comprising at least one record for each subject registered with the de-centralized healthcare system. The staff can access these records for the purpose of enhancing the quality and productivity of communication with the subject. These records can be stored by electronic and/or hard copy. The records contain information regarding, for example, one or more of personal information; address; clinical history; designated health conditions characteristic of the subject; prescriptions and regimens characteristic of the subject; a history of correspondence between the subject and the central station; a history of visits to the subject's home by the de-centralized healthcare system; a list of the facilities, systems, and devices located in the subject's home and information regarding the condition, maintenance, and warranty of the facilities, systems, and devices; emergency contacts; next of kin; and any special needs that the subject might have, for example, handicap accessibility, accommodations for diminishment or loss of sight or hearing, or a need for household assistance for hygiene and/or provision/preparation of food. Regarding issues of subject information, the systems disclosed herein comply with relevant laws and ethical standards of handling such information, for example, HIPAA.

The staff at the central station can comprise more than one, or many more than one, healthcare practitioners. A healthcare practitioner can be, for example, a doctor of medicine, a doctor of osteopathy, or a nurse practitioner. The healthcare practitioner can take appointments with the subject, be on-call for subject requests, or be available for emergency communication with the subject. In some embodiments, the healthcare practitioner has a specialty. In some embodiments, the staff comprises two or more healthcare practitioners working simultaneously, and there is a benefit wherein the two or more healthcare practitioners comprise the same specialty, complementary specialties, or distinct specialties. In cases of the same or complementary specialties, for example, a gerontologist and a nurse practitioner, the healthcare practitioners are able to collaborate on the provision of healthcare services to similar subjects. In cases of distinct specialties, the healthcare practitioners are able to provide healthcare service to different populations of subjects. The staff at the central station optionally comprises one or more healthcare providers wherein the healthcare providers are or are not healthcare practitioners.

Communication between the central station and the subject can take several forms. The communication can be a request for an appointment made by either the subject or the central station, wherein an appointment made by the central station is made by a healthcare practitioner or support personnel. The communication can also be the fulfillment of a scheduled event, wherein the communication is initiated by either the subject or the central station. The communication can also regard a routine event that happens periodically or regularly. The communication can also comprise an emergency event, wherein the communication is initiated either by the subject or by the central station, wherein the central station was alerted to the need for emergency communication by an emergency device, wherein the emergency device is optionally located in the subject's home.

During a communication between the subject and the central station, the healthcare practitioner may optionally provide a healthcare service to the subject. In some embodiments, the subject requests the healthcare service. In some embodiments, the healthcare practitioner offers the healthcare service. The healthcare practitioner can offer the healthcare service for a number of reasons, for example, that: i) the healthcare service was a scheduled event, such as an appointment; ii) the healthcare service was a routine event; iii) the central station became aware of a want or need of a healthcare service by the subject, optionally via a device located in the subject's home; or iv) a care pathway protocol applicable to the subject's designated health condition advised providing the healthcare service. In some embodiments, the service comprises a scheduled service, a routine service, or an emergency service. In some embodiments, the healthcare practitioner has a specialty and provides to the subject a healthcare service associated with the specialty, wherein the subject comprises a designated health condition that corresponds to the specialty.

Non-limiting examples of specialties applicable to any healthcare practitioner, healthcare provider, or primary care provider described herein include allerology, andrology, anesthesia, angiology, cardiology, dermatology, emergency, endocrinology, family, gastroenterology, geriatrics, gerontology, gynecology, hematology, hepatology, immunology, infectious diseases, intensive care, nephrology, neurology, neurosurgery, obstetrics, oncology, ophthalmology, oral and maxillofacial surgical, orthopedics, otolaryngology, paleopathology, palliation, pathology, pediatrics, podiatry, psychiatry, pulmonology, radiology, rehabilitation, rheumatology, serology, sexual health, sports, surgery, toxicology, transplantation, and urology.

In some embodiments, the healthcare service comprises an advice, an instruction, or a recommendation. In some embodiments, the advice, instruction, or recommendation comprises information regarding a regimen or a self-administration of a healthcare procedure. In some embodiments, the healthcare service comprises an inquiry. In some embodiments, the inquiry comprises a request for information regarding a regimen, a self-administration of a healthcare procedure, a present state of health, a mental state, an emotional state, a state of morale, or a condition of a system, device or instrument located in the subject's residence. In some embodiments, the healthcare service comprises a prescription. In some embodiments, the healthcare service comprises the discussion, explanation, or provision of the results of a test, assay, or diagnosis. In some embodiments, the subject is the owner of an animal, and the subject administers the healthcare procedure to the animal.

In some embodiments of the invention, subjects will be encouraged, instructed, or advised to follow, participate in, or live by one or more regimens. The regimens exist to improve the quality of the subject's health and life and to provide simple and lucid recommendations that the subject can easily follow after initial training by staff of the de-centralized healthcare system. Staff, or in some embodiments, healthcare practitioners, optionally communicate with the subject regarding the practice, success, convenience, enjoyment, relevance, or improvement of the regimen. In some embodiments, communication regarding a regimen comprises a questionnaire, an appointment, or an interview. Periodically, a regimen can be updated, modified, or improved for a single subject, for some subjects, or for all subjects in the de-centralized healthcare system. In some embodiments, a care pathway protocol comprises one or more regimens. A regimen can be either optional or mandatory. In some embodiments, the regimen comprises one or more of a medical regimen, an exercise regimen, a nutritional regimen, a mental aptitude regimen, a morale regimen, or a hygiene regimen. A medical regimen comprises the medication that a subject should take including dosing information, for example, amounts, frequency, time of day, with or without food, etc, and/or the medical procedures that a subject should self-administer. An exercise regimen comprises methods of maintaining the physical and cardiovascular strength of the subject, and may include recommendations for frequency, vigor, time of day, specific exercise to perform, and exercise equipment to be used, which is optionally provided by the de-centralized healthcare system. A nutritional regimen comprises the foods that a subject should eat, in some embodiments, to sustain or improve the strength of the subject or to influence or prevent changes in the subject's body weight. A nutritional regimen optionally comprises the use of specific nutritional supplements or vitamins, which are optionally provided by the de-centralized healthcare system. A mental aptitude regimen comprises methods and mental exercises for sustaining the intellectual wellness of a subject, wherein the subject optionally comprises one or more forms of dementia. A morale regimen comprises methods, advice, and encouragement directed towards improving or sustaining the emotional wellbeing of the subject. A hygiene regimen comprises methods for sustaining the physical cleanliness and topical health of the subject, optionally comprising sanitizing or disinfecting areas of the subject's body in need thereof, and optionally further comprises sustaining or improving the cleanliness and sanitation of the subject's home.

In some embodiments, the subject is educated in the self-administration of healthcare procedures. The de-centralized healthcare system provides the subject with the materials, equipment, supplies, pharmaceuticals, instruments, devices, and kits needed to perform the self-administrated procedures. Non-limiting examples of a self-administered healthcare procedure comprise a self-injection of an injectable material; a self-medication with a medication; a self-application of a topical material; a self-examination; a use of a device; a physical therapy; or a collection of a bodily fluid, a tissue or a discharge. In some embodiments, the self-administered procedure improves the condition of the subject. In some embodiments, the self-administered procedure collects information or collects a bodily fluid, a tissue, or a discharge for the purpose of finding information. In some embodiments, the information is used for a diagnosis, an observation, or a follow-up. In some embodiments, the topical material comprises a medicament that is applied to the skin, a device that is worn on the body, or an article of clothing. Non-limiting examples of a medicament that is applied to the skin include a lotion, a salve, an ointment, a cream, and an oil. The self-examination and the collection of a bodily tissue, fluid, or discharge can optionally comprise the use of a kit, which comprises some or all of the materials needed to perform the examination or collection. A kit can be accompanied by literature providing instructions for the use of the kit, or the subject can conduct an appointment with the staff at the central station to learn the proper use of the kit. A kit can contain any combination of: medications disclosed herein or suitable for providing healthcare for a designated health condition; devices disclosed herein or suitable for providing healthcare for a designated health condition; materials required to perform a regimen or self-administered healthcare service disclosed herein or suitable for providing healthcare for a designated health condition; and literature describing any aspect of the invention and/or providing instructions to the subject. All materials, devices, instruments, injectables, medication, medicaments, air/oxygen supplies, articles of clothing, and kits used in the self-administration of the healthcare procedure are optionally provided by the de-centralized healthcare system.

In some embodiments, the subject is the owner of an animal, wherein the animal is the recipient of the healthcare. In some embodiments, the animal is a mammal, a reptile, a bird, an amphibian, or an aquatic animal. In some embodiments, the animal is a mammal. In some embodiments, the animal is a high-value animal. In some embodiments, the animal is a pet, a guardian, a livestock, a service animal, a show animal, a working animal, a racing animal, or a breeding animal. The systems and methods contemplated in this invention can be applied to the health monitoring and healthcare provision of animals equally as effectively as to humans. In embodiments wherein healthcare services are provided to a non-human animal, the healthcare practitioner and provider is a veterinarian.

The de-centralized healthcare system is highly conscientious in the selection of subjects who are appropriate for registration with the de-centralized healthcare system. In some embodiments, the system selects subjects who comprise at least one designated health condition, which the system has previously determined to be compatible with the goals of the system and with at least one care pathway protocol. In some embodiments, the de-centralized healthcare system comprises staff that specialize in selecting subjects for admission to the de-centralized healthcare system. The staff that specializes in selecting subjects for admission to the de-centralized healthcare system can be a nurse practitioner.

Prior to and during the admission and registration processes, the system will consult the subject's primary care provider, who will participate in the decision of admitting or not admitting the subject into the de-centralized healthcare system. The primary care provider can provide relevant information regarding the medical status and social conditions characteristic of the subject and how the same apply to the healthcare services provided by the de-centralized healthcare system. In some embodiments, the primary care provider is involved in the healthcare services provided during the episode. In some embodiments, the primary care provider is involved in the decisions regarding the termination of the episode. In some embodiments, the primary care provider is involved in the decisions regarding the termination of the follow-up period. In some embodiments, the primary care provider is involved in the decisions regarding re-admission. In some embodiments, the primary care provider collaborates with the staff of the de-centralized healthcare system. In some embodiments, the primary care provider is involved in the delivery of healthcare services at the subject's home.

The subject lives at a location remote from any facilities that provide the management or operation of the de-centralized healthcare system, and can live in a residence that the subject recognizes as the subject's home. The subject may rent, own, borrow, or share the residence, which can be, for example, a private home, a nursing home, an apartment, a hotel, or an assisted-living facility. The subject does not reside in a hospital or a clinic that is owned, operated, or affiliated with the de-centralized healthcare system. In some embodiments, the subject is at or near the end-of-life. All subjects are registered with the de-centralized healthcare system, and the system possesses records to that effect. Persons who are not registered with, or not admitted to, the system do not have means to contact the system or the central station to request or discuss healthcare. Persons, for example, relatives, friends, and caretakers, acting on behalf of a subject who is registered with the system, especially in the case of an emergency, can contact the system or the central station using the means available to the subject. In some embodiments, the subject maintains a calendar of scheduled and/or routine events and healthcare services, and both the subject and the central station can access, modify, and request appointments for the calendar.

The de-centralized healthcare system comprises a plurality of care pathway protocols. Each care pathway protocol is applicable to at least one designated health condition, and each designated health condition has at least one care pathway protocol. Although the specific details of the care pathway protocols can vary substantially from one another, all care pathway protocols comprise instructions, and optionally regimens, that the staff of the de-centralized healthcare system and the subjects follow in the performance of healthcare services. In some embodiments, healthcare providers who are not directly affiliated with the de-centralized healthcare system also act according to one or more care pathway protocols when providing healthcare to a subject, wherein the subject is registered with, or admitted to, the de-centralized healthcare system. A care pathway protocol can comprise one or more of steps, processes, procedures, methods, medications, appointments, or use of devices, instruments, kits or facilities. Different care pathway protocols can share common elements, thus, different care pathway protocols may or may not be mutually exclusive in their respective elements. In some embodiments, a care pathway protocol comprises a series of mandatory steps, elements, or actions. In some embodiments, a care pathway protocol comprises a series of optional steps, elements, or actions. In some embodiments, a care pathway protocol comprises a series of steps, elements, or actions, wherein some of the steps, elements, or actions are mandatory and some of the steps, elements, or actions are optional. In some embodiments, the optional steps, elements, or actions are the option of the healthcare provider of the de-centralized healthcare system. In some embodiments, the optional steps, elements, or actions are the option of the subject of the de-centralized healthcare system.

In some embodiments, a care pathway protocol comprises at least one of the following elements:

a) Subject Qualification and Admission, comprising: 1) Stability Criteria, comprising: Subject status, Psychological and Social safety status; and 2) Assessments, comprising: Subject's Health, Home Assessments, and Nutritional Needs;

b) Orders, Treatments and Activities, comprising: medications, subject activities, nutritional supplementation, procedures, durable medical equipment, and optionally interventions effective to improve the health of the subject;

c) Monitoring, comprising: biometric and telecommunication equipment to monitor the subject

d) Education and Care Coordination/Integration, comprising: Instructive exercises and literature; and

e) Advanced Medical Directive, comprising: discussions regarding one or both of chronic care management and advanced care planning

In some embodiments, a care pathway protocol comprises all of the above elements. In some embodiments, a care pathway protocol consists of all of the above elements.

In some embodiments, the care pathway protocols are periodically, regularly, or frequently reviewed for efficacy and frequency of use. Care pathway protocols found to be especially effective can be identified to healthcare providers as the favored or front-line care pathway protocol for a given designated health condition. A care pathway protocol can be updated, modified, improved, or removed from the system. New care pathway protocols can be written, added to the system, or used experimentally on a sample of subjects wherein the subject has optionally consented to the use of an experimental care pathway protocol, wherein the experimental care pathway protocol is optionally evidence-based.

In some embodiments, a healthcare provider of a de-centralized healthcare system is trained in at least one care pathway protocol. In some embodiments, a healthcare provider is trained in all care pathway protocols. In some embodiments, a healthcare provider having a specialty is trained primarily in care pathway protocols corresponding to the healthcare provider's specialty. In some embodiments, a healthcare provider comprising a specialty is trained exclusively in care pathway protocols corresponding to the healthcare provider's specialty. In some embodiments, a healthcare provider having a specialty is trained both in care pathway protocols corresponding to the healthcare provider's specialty and in care pathway protocols not corresponding to the healthcare provider's specialty.

In some embodiments, healthcare providers of the de-centralized healthcare system only provide healthcare according to the care pathway protocols applicable to the designated health condition of the subject. In some embodiments, the healthcare providers of the de-centralized healthcare system can supplement a care pathway protocol with judgment based on education, training, and experience in relevant healthcare fields. In some embodiments, the system determines which care pathway protocol will be used for a subject based on the designated health condition of the subject. In some embodiments, a healthcare provider of the system makes a conscientious determination of which care pathway protocol to apply to a subject. This conscientious determination may be based on observation, subject history, or the education, training, and experience of the healthcare provider in relevant healthcare fields. In emergency situations, there is a greater likelihood that a healthcare provider invokes judgment or a conscientious determination.

A designated health condition can have one or more care pathway protocols associated with it. In some embodiments, different types of healthcare providers use the same care pathway protocols. In some embodiments, different types of healthcare provider's use different care pathway protocols. For example, a nurse, a paramedic, and a doctor of medicine may follow different care pathway protocols for providing healthcare to the same subject for the same designated health condition. In some embodiments, if more than one care pathway protocol exists for a specific designated health condition, the care pathway protocols are effective to be used independently of one another. In some embodiments, more than one care pathway protocols are effective to be used synergistically with one another, meaning that different care pathway protocols can be used together to provide benefits that are cumulative or complementary. The synergistic use of care pathway protocols can be done by a single healthcare provider or by multiple healthcare providers. In the case of multiple healthcare providers, in some embodiments, each healthcare provider performs a single care pathway protocol, and in some embodiments, more than one healthcare provider can collaborate on at least one care pathway protocol. Care pathway protocols used synergistically can be used simultaneously, sequentially, or at different times. In some embodiments, care pathway protocols are designed to be used synergistically. In some embodiments, new care pathway protocols are written for the purpose of being used synergistically with an existing care pathway protocol.

The communication system is an integral feature of the de-centralized healthcare system, in that the communication system allows the subject to reside at a location remote from the central station. The communication system ensures the continued correspondence between the subject and the system and allows the delivery of healthcare services to the subject wherein the subject is in need or want thereof. In some embodiments, the communication system comprises whereby the healthcare practitioner provides a healthcare service to the subject. In some embodiments, the communication system is effective to promote a diagnosis, an evaluation, or an assessment of the subject by the healthcare practitioner at the central station. Thus, hospital-quality observations of the subject can be made from a remote location. The communication system provides a two-way system, wherein either the subject or the central station can initiate or receive communication. In some embodiments, the communication system can be activated and deactivated by the central station or the subject. In some embodiments, the communication system is always active. In some embodiments, the central station can activate or inactivate the communication system in the subject's home. In some embodiments, the communication system comprises more than one component. In some embodiments, the communication system comprises more than one component, optionally wherein each individual component can be activated or deactivated independently by either the subject or the central station.

In some embodiments, the communication system comprises one or more of written, audio, visual, monitoring, or audio/visual communication. Written communication comprises one or more of handwritten or typed communication, for example, e-mail, electronic chat, dictation, or notes taken by the subject and sent to the central station. Non-limiting examples of audio communication comprise telephone, telecom, and audio signaling, such as alerts or alarms, which bring information to the attention of either the subject or the central station, for example, new, or emergency information. Non-limiting examples of visual communication provide one-way viewing of the subject by the central station or two-way viewing between the subject and the central station, for example, by a camera, a webcam, streaming video, or still-photography; illustrative communication, for example, to visualize instructions for a self-administered healthcare service; or sign-language communication for subjects with limited hearing or hearing loss. In some embodiments, the communication system comprises audio/visual communication. An audio/visual communication system optionally embodies some or all of the features of all other communication systems described herein, and provides the subject with the satisfaction and confidence of an in-person meeting with a healthcare practitioner. In some embodiments, subjects and healthcare practitioners are encouraged to participate in audio/visual communication.

In some embodiments, the communication system allows monitoring. Monitoring of the subject allows the central station to remain aware of the status of the subject at all times, including times when a subject is asleep, non-communicative, or unwilling or unable to correspond.

Monitoring of the subject for a designated health condition by the central station is equally as effective as the monitoring of a subject for the same designated health condition in a hospital. In some embodiments, monitoring of the subject for a designated health condition by the central station is more effective than monitoring by a hospital. Monitoring of the subject comprises the use of one or more of audio surveillance, video surveillance, journalistic entries by the subject, or a use of a device. Audio and video surveillance allow the central station to observe the subject and make observations on, for example, the subject's status, health, morale, nutrition, exercise, and sleep habits or regimens. Audio and video surveillance allow the central station to become aware of emergency situations, for example, a fall or a wound, even in situations wherein the subject is unable to initiate communication with the central station. Journalistic entries by the subject allow the central station to monitor the periodic activities of the subject and optionally gain insight into the mental aptitude and emotional wellbeing of the subject. Non-limiting examples of devices used for the monitoring of a subject comprise a medical device, a positioning device, or an emergency device. A medical device may be worn on the body or situated in the residence of the subject and used to make observations of the subject and communicate the data to the central station.

Non-limiting examples of devices used for monitoring the subject comprise glucose meters, pulse oximeter, weight scales, blood pressure cuffs, electrocardiographic (EKG) devices, stethoscopes, two-way audio/video devices, wireless hubs, computers, inclinometers, motion detectors, personal emergency response units, bed sensors, activities of daily life (ADL) monitors, biometric skin sensors, opthalmoscopes, otoscopes, ultrasound/echocardiogram devices, impedance monitors, veterinary equipment, and temperature-reading devices. The devices allow the central station to monitor the health and vital signs of the subject. Non-limiting examples of a positioning device comprise a tracking device or a global positioning device. A tracking device allows the central station to determine where the subject is presently situated in the residence. A global positioning device allows the central station to determine if the subject has left the residence, and if so, where the subject can be found. The global positioning device is particularly helpful should the subject suffer an emergency while outside the home and require specific assistance such as at-home healthcare, emergency care, or transportation to the residence or to a local healthcare facility. An emergency device, such as a personal emergency response unit, can alert the central station to the subject's need or want for emergency care, for example, wherein the subject is physically unable to communicate by other means, or cannot reach or access an audio, visual, or audio/visual communication system.

In some embodiments, the invention contemplates a de-centralized healthcare system comprising a dispatch system, wherein the dispatch system comprises a staff with at least one healthcare provider. In some embodiments, the dispatch system is staffed by at least one healthcare provider. The dispatch system is located at a site effective to send healthcare providers directly to the residence of a subject, wherein the subject is in need or want of at-home healthcare services. In this capacity, the dispatch system is complementary to the central station and provides the subject with the in-person healthcare that is often required or desired for the effective treatment of a designated health condition. In some embodiments, the de-centralized healthcare system comprises both a central station and a dispatch system. The dispatch system can be located in the same facility as the central station, or in a different facility. There is constant communication between the central station and the dispatch system to ensure that healthcare providers are dispatched to the subject promptly with the most accurate information.

Non-limiting examples of healthcare providers comprise a physician, a clinician, a nurse, a paramedic, a therapist, and a pharmacist, wherein the physician is, for example, a doctor of medicine, osteopathy, chiropractic, podiatry, optometry, or dentistry. In some embodiments, the healthcare provider is available by appointment. In some embodiments, the healthcare provider is on-call. In some embodiments, the healthcare provider is available for emergencies. In some embodiments, a healthcare practitioner is a healthcare provider. In some embodiments, a healthcare practitioner and a healthcare provider are mutually exclusive. In some embodiments, a healthcare provider specializes in one or both of remote healthcare services via the central station or at-home healthcare service via the dispatch system.

The dispatch system dispatches one or more healthcare providers to the subject to provide healthcare services. Two exemplary methods by which a healthcare provider is dispatched to the subject: i) wherein the subject requested the dispatch, and ii) wherein the subject did not request the dispatch.

Cases wherein the subject requested the dispatch comprise scheduled and emergency services. Subjects can request scheduled events, for example, appointments with healthcare providers by contacting the central station for an at-home appointment. Subjects can request emergency service by contacting the central station through a communication system, for example, an audio, visual, audio/visual, or written system, or via an emergency device.

Cases wherein the subject did not request the dispatch comprise scheduled, routine, and emergency services. The central station can make or request a scheduled event, such as an in-home appointment, with the subject via the communication system. Routine services, such as routine entry into the subject's home to perform a task or healthcare service, can be added to the subject's calendar by the central station and performed, in some embodiments, with, or, in some embodiments, without, the participation of the subject. Emergency services that were not requested by the subject are initiated, for example, by an observation made by the central station or a signal received from a device in the subject's home alerting the central station to a need for an emergency healthcare service.

In some embodiments, the de-centralized healthcare system of the invention comprises a number of emergency protocols. An emergency protocol can be initiated by the subject, or a friend, relative, or caretaker thereof, or by the central station. Non-limiting examples of emergencies contemplated by the emergency protocols of the present invention include the inability to breathe; labored breathing; moderate, severe, or crushing chest pain or discomfort; inability to move all or part of the body; unconsciousness/unresponsiveness; likelihood of losing consciousness; profound bleeding; bloody vomit; seizure; severe burns; choking; confusion/disorientation; weakness; fever; falling or nearly falling; increasing, unexplained, or intolerable pain; severe headache; allergic reaction; rash; nausea; vomiting; diarrhea; bloody, burning, or increased urine; irregular pulse; palpitations; depression; extreme, sudden, or unexplained sadness; and suicidal, self-harming or self-hating thoughts.

In some embodiments, the scheduled healthcare service or the routine healthcare service comprises an appointment, an examination of the subject, an examination of a device, a check-up, a follow-up, a visit, an adjustment, an injection, an infusion, or a delivery or a pick-up of an item. Devices are periodically inspected for efficacy and condition, and can be repaired, replaced, refurbished, or updated as needed or desired. Devices or other items worn by the subject or mounted or affixed within the residence can be physically adjusted. In some embodiments, a technical support personnel participates in the inspection or adjustment. Non-limiting examples of an item include a medication, a vitamin, a dietary supplement, an article of clothing, a device, a report, a questionnaire, a kit, and a sample, wherein the sample comprises a body fluid, a tissue, or a discharge.

Non-limiting examples of emergency healthcare services provided by the healthcare provider comprise a resuscitation, a defibrillation, an injection, a rescue from a sudden condition, or a transporting of the subject to a clinic or a hospital. Non-limiting examples of a sudden condition comprise a heart attack, a stroke, an instance of shock, a loss of blood pressure, a loss of consciousness, a fall, a wound, an adverse reaction to medication, an allergic reaction, or a failure of a device. In some embodiments, the healthcare provider arrives at the residence aware of the nature of the emergency and aware of the methods used to provide healthcare services for the emergency, as communication between the subject and the healthcare provider might be impossible.

When the healthcare provider arrives at the residence, the healthcare provider acts according to the most relevant care pathway protocols for the designated health condition embodied by the subject. The healthcare provider, or in some embodiments, more than one healthcare provider, performs one or more care pathway protocols, which can be performed independently or synergistically, and make all appropriate judgments and conscientious determinations as described herein.

In some embodiments, a healthcare provider and a healthcare practitioner collaborate in providing one or more healthcare services to a subject, wherein the healthcare practitioner at the central station communicates with the subject and/or the healthcare provider via the communication system in the subject's residence. In some embodiments, the healthcare provider and the healthcare practitioner perform the same care pathway protocol. In some embodiments, the healthcare provider and the healthcare practitioner perform different care pathway protocols independently. In some embodiments, the healthcare provider and the healthcare practitioner perform different care pathway protocols synergistically. In some embodiments, care pathway protocols are designed to be performed via the collaboration of a healthcare practitioner and a healthcare provider.

In some embodiments, the subject's home optionally comprises means of entry by the healthcare practitioner. Non-limiting examples of the means of entry include a key, an access code, an access card, or an audio, visual, or audio/visual intercom between the subject and the entry to the subject's residence.

In some embodiments, the invention contemplates a de-centralized healthcare system comprising a plurality of kits. In some embodiments, the de-centralized healthcare system comprises a central station, a dispatch system, and a plurality of kits. Each kit independently comprises materials effective for self-administration of a healthcare service. Kits can be delivered to the subject either by the de-centralized healthcare system or via a typical parcel carrier, for example, the United States Post Office. Each kit serves a specific purpose and enables the self-administration of a specific healthcare service. In some embodiments, the subject uses the kit to collect a bodily tissue, a fluid, or a discharge. In some embodiments, the kit is diagnostic. In some embodiments, the use of the kit by the subject produces a result, for example, data, or a bodily tissue, a fluid, or a discharge. The subject might require assistance in the use of a kit, especially when a certain kit is being used by the subject for the first time. Thus, the subject can contact the central station to discuss the proper use of the kit with a healthcare practitioner. In some embodiments, the kits comprise instruction manuals, wherein the instruction manuals optionally comprise illustrations.

In some embodiments, the invention contemplates a de-centralized healthcare system comprising a laboratory. In some embodiments, the de-centralized healthcare system comprises a central station, a dispatch system, and a laboratory. In some embodiments, the de-centralized healthcare system comprises a central station, a dispatch system, a plurality of kits, and a laboratory. The laboratory comprises facilities effective to perform experiments and analyses relevant to determining, monitoring, diagnosing, sustaining, or improving the health of the subject. In some embodiments, the laboratory analyzes the kits used by the subject. In some embodiments, the laboratory performs an experiment or assay on a sample provided by the subject, wherein the sample comprises a bodily tissue, a fluid, or a discharge. In some embodiments, the laboratory analyzes data provided by the subject. In some embodiments, the de-centralized healthcare system owns the laboratory. In some embodiments, the laboratory is affiliated or associated with the de-centralized healthcare system. In some embodiments, the laboratory is contracted by the de-centralized healthcare system. In some embodiments, the laboratory is a component of a conventional healthcare facility cooperating with the de-centralized healthcare system.

Many subjects registered with the de-centralized healthcare system are subjects of limited physical capacities. In some embodiments, the subject is not ambulatory. In some embodiments, the subject is wheelchair-bound. In some embodiments, the subject is physically weak, aged, atrophied, decrepit, or lacks ordinary motor skills or functions. In some embodiments, the subject is a shut-in. For the benefit of subjects of limited physical means, and to ensure the health, happiness, and general wellbeing of such subjects, some embodiments of the invention contemplate providing services for the maintenance of the subject's residence.

In some embodiments, the invention contemplates a de-centralized healthcare system comprising a hygiene system. In some embodiments, the de-centralized healthcare system comprises a central station, a dispatch system, and a hygiene system. In some embodiments, the de-centralized healthcare system comprises a central station, a dispatch system, a plurality of kits, a laboratory, and a hygiene system. In some embodiments, the hygiene system is staffed by at least one hygienist.

In some embodiments, a subject is physically unable to perform essential day-to-day hygiene duties. Non-limiting examples of hygiene duties comprise laundry, cleaning bed sheets, sanitizing, and disinfecting. In some embodiments, a hygienist enters the home of the subject to perform various hygiene duties as needed or desired. The hygienist can enter the subject's residence to perform hygiene duties, for example, daily, semi-weekly, weekly, biweekly, or monthly.

In some embodiments, the invention contemplates a de-centralized healthcare system comprising a food delivery system. In some embodiments, the de-centralized healthcare system comprises a central station, a dispatch system, and a food delivery system. In some embodiments, the de-centralized healthcare system comprises a central station, a dispatch system, a hygiene system, and a food delivery system. In some embodiments, the de-centralized healthcare system comprises a central station, a dispatch system, a plurality of kits, a laboratory, a hygiene system, and a food delivery system. In some embodiments, the subject is incapable of providing food. The food delivery system provides food or a food supply to the subject, for example, daily, semi-weekly, weekly, biweekly, or monthly. Non-limiting examples of food or a food supply comprise prepared meals, packaged foods, frozen foods, groceries, nutritional supplements, vitamins, or a nutritional regimen, wherein the nutritional regimen is provided by the de-centralized healthcare system.

The de-centralized healthcare systems of the invention disclosed herein also contemplate systems wherein the de-centralized healthcare system affiliates, collaborates, communicates, or cooperates with conventional healthcare facilities. The affiliation, collaboration, communication, or cooperation with conventional healthcare facilities allows the de-centralized healthcare system to, for example, access records, staff, and persons with potential to become admitted subjects to the de-centralized healthcare system. This integration of the conventional healthcare facility into the de-centralized healthcare system enhances the communication of medical records and assists subjects in moving their episodes of acute care from, for example, a hospital to their own homes.

The invention described herein also contemplates methods of providing healthcare via one or more of the systems described herein. In some embodiments of the methods, a de-centralized healthcare system supplies a subject with the means to perform a self-administered healthcare service. Non-limiting examples of means suitable to perform a self-administered healthcare service include kits, syringes, needles, IV bags, tubing, medications, nutritional supplements, vitamins, bandages, patches, lotions, ointments, creams, diagnostic devices, therapeutic devices, and monitoring devices; any of the foregoing devices can be provided with instructions for suitable use by a person with no special clinical training or experience. In some embodiments, the means suitable to perform a self-administered healthcare service are effective for self-administration of a healthcare service. In some embodiments of the methods, the healthcare services are provided remotely via the communication system or at-home.

The invention described herein also contemplates methods of selecting a subject for a de-centralized healthcare system. The selection of subjects is related to the efficacy of the system, as the subject should be capable of experiencing equal or better health in the de-centralized healthcare system than in a conventional healthcare facility. In some embodiments, the subject is at or near the end-of-life. In some embodiments, the subject is selected based on a diagnosis of the subject and subsequent comparison of the diagnosis to a list of designated health conditions. In some embodiments, a physician employed by the system makes the diagnosis. In some embodiments, the diagnosis was made previously by the subject's private physician, and was optionally verified by a physician associated with the de-centralized healthcare system. In some embodiments, the subject is selected by determining the costs and benefits of at-home living versus conventional healthcare facility living for the subject. In some embodiments, the costs and benefits comprise quality of health and quality of life costs and benefits. In some embodiments, the costs and benefits comprise emotional costs and benefits, for example, the personal happiness of the subject. In some embodiments, the costs and benefits comprise economic costs and benefits to both the subject and the system. In some embodiments, selecting a subject comprises all of the factors described herein. Once a subject has been selected, the subject becomes registered with the system, and records for the subject are created at the central station.

Non-limiting examples of designated health conditions include: asthma/bronchitis, cellulitis, chronic heart failure (CHF), chronic obstructive pulmonary disease (COPD), deep venous thrombosis, diverticulitis, disequilibrium (vertigo), gastroenteritis/esophagitis, kidney and urinary tract infection, pneumonia, dehydration, viral illness, diversion diagnosis, post-op central nervous system (CNS) procedures, post-op cardiac bypass, post-op orthopedic procedures, medical back problems, strokes, seizures, neoplasms and lymphoma related hospitalizations, chronic infections, major cardiac surgery, coronary bypass surgery, permanent cardiac pacemaker, vascular procedures, post-myocardial infarction (MI), gastrointestinal (GI) procedures (small and large bowel), pulmonary embolism, respiratory failure with vent support, specific end-stage disease states, ventilator care, external feeding care, New York Heart Association (NYHA) class IV heart failure, severe/symptomatic valvular heart disease in individuals who are poor surgical candidates, Global Initiative for Chronic Obstructive Lung Disease (GOLD) classification stage IV COPD/respiratory failure, malignant disease, failure to thrive of senescence, acquired immunodeficiency syndrome (AIDS), cirrhosis in individuals who are not candidates for transplantation, and amyotrophic lateral sclerosis.

Non-limiting examples of designated health conditions include: heart transplant or implant of heart assist system; craniotomy and endovascular intracranial procedures; extracranial procedures; multiple sclerosis and cerebellar ataxia; intracranial hemorrhage or cerebral infarction; transient ischemia; nonspecific cerebrovascular disorders; seizures; headaches; disequilibrium (vertigo); major chest procedures; other respiratory system operating room procedures; pulmonary embolism; respiratory infections and inflammations; respiratory neoplasms; pulmonary edema and respiratory failure; chronic obstructive pulmonary disease; simple pneumonia and pleurisy; bronchitis and asthma; respiratory signs and symptoms; respiratory system diagnosis with ventilator support; deep venous thrombosis, chronic heart failure; cardiac valve and other major cardiothoracic procedures; cardiac defibrillator implant with cardiac catheterization with acute myocardial infarction/heart failure/shock; cardiac defibrillator implant without cardiac catheterization with major coronary bypass with percutaneous transluminal coronary angioplasty; major cardiovascular procedures or thoracic aorta; permanent cardiac pacemaker implant; percutaneous cardiovascular procedure with drug-eluting stent or other circulatory system operating room procedures; other vascular procedures; cardiac pacemaker revision except device replacement; other circulatory system operating room procedures; acute myocardial infarction; circulatory disorders except acute myocardial infarction, with cardiac catheterization; heart failure and shock; deep vein thrombophlebitis; dehydration; diversion diagnosis; end-stage disease states; external feeding; New York Heart Association class IV heart failure; severe/symptomatic valvular heart disease in individuals who are poor surgical candidates; Global Initiative for Chronic Obstructive Lung Disease classification stage IV COPD/respiratory failure; malignant disease; failure to thrive of senescence; acquired immunodeficiency syndrome (AIDS); cirrhosis in individuals who are not candidates for transplantation; amyotrophic lateral sclerosis; peripheral vascular disorders; atherosclerosis; hypertension; cardiac arrhythmia and conduction disorders; syncope and collapse; chest pain; other circulatory system diagnoses; diverticulitis; post-operative central nervous system procedures; strokes; neoplasms and lymphoma-related hospitalizations; chronic infections; stomach, esophageal and duodenal procedures; major small and large bowel procedures with other digestive system operating room procedures; peritoneal adhesiolysis; hernia procedures except inguinal and femoral; other digestive system operating room procedures; major gastrointestinal disorders and peritoneal infection; digestive malignancy; gastrointestinal hemorrhage; complicated peptic ulcer; gastrointestinal obstruction; esophagitis, gastroenterological and miscellaneous digestive disorders; other digestive system diagnoses; laparoscopic cholecystectomy without common bile duct exploration; cirrhosis and alcoholic hepatitis; malignancy of hepatobiliary system or pancreas; disorders of pancreas except malignancy; disorders of liver except malignancy, cirrhosis, alcoholic hepatitis; disorders of the biliary tract; spinal fusion except cervical; wound debridement and skin graft except hand, for musculoskeletal-connective tissue; revision of hip or knee replacement; major joint replacement or reattachment of lower extremity; hip and femur procedures except major joint; knee procedures with past diagnosis of infection; back and neck procedures except spinal fusion or disctectomy; lower extremety and humer procedures except hip, foot, femur; other musculoskeletal system and connective tissue operating room procedures; fractures of hip and pelvis; osteomyelitis; pathological fractures and musculoskeletal and connective tissue fractures, sprains, strains and dislocations except femur, hip, pelvis and skin graft and/or debridement for skin ulceration or cellulitis; connective tissue disorders; medical back problems; tendonitis, myositis and bursitis; fracture, sprain, strain and dislocation except femur, hip, and pelvis; skin graft and/or debridement for skin ulcer or cellulitis; other skin, subcutaneous tissue and breast procedures; skin ulcers; cellulitis; skin grafts and wound debridement for endocrine, nutrition, and metabolic disorders; other endocrine, nutrition and metabolic operating room procedures; diabetes; nutritional and miscellaneous metabolic disorders; endocrine disorders; major bladder procedures; kidney and ureter procedures for neoplasm; transurethral procedures; other kidney and urinary tract procedures; renal failure; kidney and urinary tract infections; urinary stones without electronic shockwave lithotripsy; other kidney and urinary tract diagnoses; major male pelvic procedures, conization, laparoscopy and tubal interruption; other female reproductive system operating procedures; lymphoma and non-acute leukemia; infectious and parasitic diseases with operating room procedure; postoperative or post-traumatic infections within operating rooms; fever; viral illness; septicemia or severe sepsis without mechanical ventilation for at least 96 hours; poisoning and toxic effects of drugs; complications of treatment; signs and symptoms; HIV with major related conditions; extensive operating room procedure unrelated to principal disorder; and non-extensive operating room procedure unrelated to principal disorder.

In some embodiments, any designated health condition described herein is contemplated with complications/comorbidities or without complications/comorbidities. In some embodiments, any designated health condition described herein is contemplated with major complications/major comorbidities or without major complications/major comorbidities. In some embodiments, any designated health condition described herein is contemplated with complications/comorbidities and with major complications/major comorbidities. In some embodiments, any designated health condition described herein is contemplated without complications/comorbidities and without major complications/major comorbidities. In some embodiments, any designated health condition described herein is contemplated with complications/comorbidities and without major complications/major comorbidities. In some embodiments, any designated health condition described herein is contemplated without complications/comorbidities and with major complications/major comorbidities.

In some embodiments, the subject is in late-term pregnancy, and receives healthcare suitable to sustain health and promote post-delivery wellness for both mother and child.

In some embodiments, the de-centralized healthcare system of the invention further comprises a dispatch system.

In some embodiments, the de-centralized healthcare system of the invention further comprises a plurality of kits, wherein each kit independently comprises materials effective for self-administration of a healthcare service.

In some embodiments, the de-centralized healthcare system of the invention further comprises a laboratory.

In some embodiments, the de-centralized healthcare system of the invention further comprises a hygiene system.

In some embodiments, the de-centralized healthcare system of the invention further comprises a food delivery system.

In some embodiments, the invention contemplates a de-centralized healthcare system for providing healthcare to one or more subjects, the system comprising: a) a central station comprising a healthcare practitioner; b) a subject, wherein the subject resides at a location remote from the central station and is registered with the de-centralized healthcare system; c) a plurality of care pathway protocols, wherein each care pathway protocol comprises a protocol for providing healthcare for a selected designated health condition to a subject in need or want thereof; and d) a communication system comprising communication between the subject and the healthcare practitioner.

In some embodiments, the central station is effective to monitor the subject. In some embodiments, the central station is effective to monitor the health of the subject. In some embodiments, the central station further comprises a record-keeping system. In some embodiments, the record-keeping system comprises at least one record for each subject registered with the de-centralized healthcare system. In some embodiments, the healthcare practitioner has access to the record for each subject registered with the de-centralized healthcare system. In some embodiments, the central station further comprises a computing system. In some embodiments, the central station further comprises a library of health reference materials. In some embodiments, the central station comprises at least two healthcare practitioners. In some embodiments, each of the at least two healthcare practitioners independently comprise a specialty. In some embodiments, the specialties comprise the same specialty, complementary specialties, or distinct specialties.

In some embodiments, the healthcare practitioner comprises a doctor of medicine, a doctor of osteopathy, or a nurse practitioner. In some embodiments, the healthcare practitioner is available by appointment. In some embodiments, the healthcare practitioner is on-call. In some embodiments, the healthcare practitioner is available for emergencies. In some embodiments, the healthcare practitioner provides a healthcare service to the subject. In some embodiments, the healthcare practitioner comprises a specialty. In some embodiments, the subject comprises a designated health condition. In some embodiments, the healthcare practitioner provides a healthcare service to the subject, wherein the specialty corresponds to the designated health condition.

In some embodiments, the healthcare practitioner is a veterinarian. In some embodiments, the subject comprises the owner of an animal. In some embodiments, the animal is in need of healthcare or the owner is in want of healthcare for the animal. In some embodiments, the animal is a pet, a guardian, a livestock, a service animal, a show animal, a working animal, a racing animal, or a breeding animal. In some embodiments, the animal is a mammal.

In some embodiments, the subject resides at a private home, a nursing home, an apartment, a hotel, or an assisted-living facility. In some embodiments, the subject comprises a designated health condition.

In some embodiments, the subject requests a healthcare service. In some embodiments, the subject requests a healthcare service via the communication system. In some embodiments, the healthcare service is a scheduled healthcare service, a routine healthcare service, or an emergency healthcare service. In some embodiments, the healthcare service comprises an advice, an instruction, or a recommendation. In some embodiments, the advice, instruction, or recommendation regards one or more of a medical regimen, an exercise regimen, a nutritional regimen, a mental aptitude regimen, a morale regimen, a hygiene regimen, or a self-administration of a healthcare procedure. In some embodiments, the self-administration of a healthcare procedure comprises a self-injection of an injectable material, a self-medication with a medication, a self-application of a topical material, a self-examination, a use of a device, a physical therapy, or a collection of a bodily fluid, a tissue or a discharge. In some embodiments, the topical material comprises a lotion, a salve, an ointment, a cream, an oil, an article of clothing, or a device worn on the body. In some embodiments, the injectable material, the medication, the topical material, or the device is provided by the de-centralized healthcare system. In some embodiments, the self-examination comprises the use of a kit provided by the de-centralized healthcare system. In some embodiments, the collection of a bodily fluid, a tissue, or a discharge comprises the use of a kit provided by the de-centralized healthcare system. In some embodiments, the subject is at or near the end-of-life. In some embodiments, the subject is the owner of an animal. In some embodiments, the animal is in need of healthcare or the owner is in want of healthcare for the animal. In some embodiments, the animal is a pet, a guardian, a livestock, a service animal, a show animal, a working animal, a racing animal, or a breeding animal. In some embodiments, the animal is a mammal. In some embodiments, the healthcare practitioner is a veterinarian. In some embodiments, each care pathway protocol of the plurality of care pathway protocols is independently effective to provide healthcare to the subject for one or more designated health conditions.

The invention contemplates a number of regimens that a subject can undertake. Regimens can be practiced alone or with the instruction of a healthcare provider. Non-limiting examples of regimens that a subject undertakes include self-monitoring, occupational therapy, speech therapy, social service, home health aide, hospice, wound care, medication management, complication prevention, diet, weight management, fluid management, exacerbation prevention, oxygen therapy, health education, breathing techniques, diabetic counseling, pain management, and infusion with one or more of antibiotics, hydration, steroids, saline, or nutrition.

Non-limiting examples of nutritional/dietary regimens include normal diet, low-salt diet, low sugar/diabetic diet, low fat/low cholesterol diet, renal (low protein/low potassium) diet, high protein/high calorie diet, low carbohydrate/calorie restricted diet, liquid diet, fluid restricted diet, ground food diet, soft pureed food diet, thickened liquid diet, and clear liquid diet. The nutritional/dietary regimen can be modified as is appropriate for the subject.

In an example of a self-monitoring regimen, a subject takes self-observations and records the results. The subject checks and records each of blood pressure, body temperature, oxygen levels, and glucose levels in the morning, afternoon, early evening, and late evening, and records the same. The subject takes and records the taking of appropriate medications at these times. The subject checks and records body weight in the morning, and exercises as tolerated in the morning.

A subject can have a regimen of light exercise, low impact exercise; rest; elevation of an extremity; measurement of the girth of an extremity; or exercising an extremity.

In some embodiments, a subject takes medications. Medications can include, for example, furosemide; methylprednisolone; ceftriaxone; normal saline; levofloxacin; vancomycin; enoxaparin; and nebulizers. Non-limiting examples of medications used in the present invention include torsemide; zaroxolyn; hydrochlorothiazide; enalapril; lisinopril; valsortan; losortan; asprin; carvedilol; metoprolol; spironolactone; digoxin; heparin; isosorbide mononitrate; hydralazine; albuterol; ipratropium; tiotropium; prednisone; zithromax; ceftriaxone; phenergan with codeine; acetylcysteine; lovenox; coumadin; azithromycin; moxifloxacin; guaifenesin; furosemide; methylprednisolone; ceftriaxone normal saline levofloxacin vancomycin; enoxaparin; and nebulizers. In some embodiments, a healthcare practitioner determines what medications a subject takes guided by the best practice of medicine. In some embodiments, a healthcare practitioner prescribes any medication known to one of skill in the art to be effective for the subject's condition. For examples of medications known to one of skill in the art, see: Goodman and Gilman's THE PHARMACOLOGICAL BASIS OF THERAPEUTICS, 11th Ed. (The McGraw-Hill Companies, Inc. 2006) (1941); THE MERCK INDEX, 14th Ed. (Merck and Co. 2006) (1889); THE MERCK VETERINARY MANUAL, 9^{th} Ed. (Merck and Co. 2005) (1955); REMINGTON: THE SCIENCE AND PRACTICE OF PHARMACY, 21st Ed. (Lippincott Williams and Wilkins 2005) (1886); and 2009 PHYSICIANS' DESK REFERENCE (Thomson Reuters 2008), each of which is incorporated by reference herein in its entirety for the teachings of medications and uses thereof.

Any of the medications can be administered orally, subcutaneously, intramuscularly, transdermally, anally, nasally, intravenously, intraperitoneally, or by infusion. In some embodiments, the medication is administered intravenously. The amount of medication given to a subject per dose is determined by a healthcare practitioner. In some embodiments, a subject is further administered a coagulant, or an anti-coagulant. In some embodiments, a subject is administered a medication known by one of skill in the art to be suitable for providing therapy to a subject having a certain designated health condition. In some embodiments, a healthcare practitioner determines the method of administration guided by the best practice of medicine. For examples of methods of administration known to one of skill in the art, see: Goodman and Gilman's THE PHARMACOLOGICAL BASIS OF THERAPEUTICS, 11th Ed.; THE MERCK INDEX, 14th Ed.; THE MERCK VETERINARY MANUAL, 9^{th} Ed.; REMINGTON: THE SCIENCE AND PRACTICE OF PHARMACY, 21st Ed.; and 2009 PHYSICIANS' DESK REFERENCE, each of which is incorporated by reference herein in its entirety for the teachings of methods of administration.

In some embodiments, each designated health condition has at least one care pathway protocol. In some embodiments, a designated health condition has one care pathway protocol.

In some embodiments, a designated health condition has more than one care pathway protocol. In some embodiments, each of the care pathway protocols is used independently. In some embodiments, the healthcare practitioner performs at least one of the care pathway protocols. In some embodiments, the more than one care pathway protocols are used synergistically. In some embodiments, the healthcare practitioner performs more than one care pathway protocol.

In some embodiments, the communication system is effective to enable a diagnosis, an evaluation, or an assessment of the subject by the healthcare practitioner. In some embodiments, the communication system comprises one or more of written, audio, visual, monitoring, or audio/visual communication. In some embodiments, the monitoring comprises one or more of audio surveillance, video surveillance, journalistic entries by the subject, or a use of a device. In some embodiments, the device comprises a medical device, a positioning device, or an emergency device. In some embodiments, the positioning device comprises a tracking device or a global positioning device. In some embodiments, the communication system is whereby the healthcare practitioner provides a healthcare service to the subject. In some embodiments, the healthcare practitioner provides a healthcare service to the subject according to a care pathway protocol. In some embodiments, the first healthcare service comprises one or more of an advice, an instruction, a recommendation, an inquiry, or a prescription. In some embodiments, the advice, instruction, recommendation, inquiry, or prescription regards one or more of a medical regimen, an exercise regimen, a nutritional regimen, a mental aptitude regimen, a morale regimen, a hygiene regimen, or a self-administration of a healthcare procedure. In some embodiments, the self-administration of a healthcare procedure comprises a self-injection of an injectable material, a self-medication with a medication, a self-application of a topical material, a self-examination, a use of a device, a physical therapy, or a collection of a bodily fluid, a tissue or a discharge. In some embodiments, the topical material comprises a lotion, a salve, an ointment, a cream, an oil, an article of clothing, or a device worn on the body. In some embodiments, the injectable material, the medication, the topical material, or the device is provided by the de-centralized healthcare system. In some embodiments, the self-examination comprises the use of a kit provided by the de-centralized healthcare system. In some embodiments, the collection of a bodily fluid, a tissue or a discharge comprises the use of a kit provided by the de-centralized healthcare system.

In some embodiments, the de-centralized healthcare system further comprises a dispatch system. In some embodiments, the dispatch system is located at the same facility as the central station. In some embodiments, the dispatch system is located at a different facility than the central station. In some embodiments, the dispatch system further comprises at least one healthcare provider. In some embodiments, the at least one healthcare provider comprises a physician, a clinician, a nurse, a paramedic, a therapist, a pharmacist, or a veterinarian. In some embodiments, the physician comprises a doctor of medicine, osteopathy, chiropractic, podiatry, optometry, or dentistry. In some embodiments, the dispatch system dispatches the healthcare provider to the subject, wherein the subject is in need or want of healthcare.

In some embodiments, the healthcare provider provides a healthcare service to the subject according to a care pathway protocol. In some embodiments, the subject requests wherein the dispatch system dispatches the healthcare provider to the subject. In some embodiments, the subject had not previously requested that the dispatch system dispatch the at least one healthcare provider to the subject. In some embodiments, the at least one healthcare provider provides a healthcare service comprising a scheduled healthcare service, a routine healthcare service, or an emergency healthcare service. In some embodiments, the subject requests the emergency healthcare service. In some embodiments, a monitoring device alerts the central station to a need for the emergency healthcare service. In some embodiments, the emergency healthcare service comprises a resuscitation, a defibrillation, an injection, a rescue from a sudden condition, or a transporting of the subject to a clinic or a hospital. In some embodiments, the sudden condition comprises a heart attack, a stroke, an instance of shock, a loss of blood pressure, a loss of consciousness, a fall, a wound, an adverse reaction to medication, an allergic reaction, or a failure of a device. In some embodiments, the scheduled healthcare service or the routine healthcare service comprises an appointment, an examination of the subject, an examination of a device, a check-up, a follow-up, a visit, an adjustment, or a delivery or a pick-up of an tem. In some embodiments, the item comprises a medication, a vitamin, a dietary supplement, an article of clothing, a device, a report, a questionnaire, a kit, or a sample, wherein the sample comprises a body fluid, a tissue, or a discharge.

In some embodiments, the de-centralized healthcare system further comprises a plurality of kits, wherein each kit independently comprises materials effective for self-administration of a healthcare service.

In some embodiments, the de-centralized healthcare system further comprises a laboratory. In some embodiments, the laboratory comprises facilities for performing an analysis of a sample provided by the subject. In some embodiments, the laboratory is independent of the de-centralized healthcare system and cooperates with the de-centralized healthcare system.

The laboratory can perform any clinical test known to one of skill in the art. Non-limiting examples of laboratory tests include basic metabolic panel (BMP); complete blood count (CBC); hemaglobin (Hgb); hematocrit (Hct); white blood cell (wbc); neutrophils (neu); lymphocytes (lym); platelet count (plt); prothrombin time (PT); international normalized ratio (INR); partial thromboplastin time (PTT); sodium chemistry; chloride chemistry; potassium chemistry; carbon dioxide chemistry; blood urea nitrogen (BUN) chemistry; creatinine chemistry; glomerular filtration rate (GFR) chemistry; glucose chemistry; troponin; creatine phosphokinase; D-dimer; B-type natriuretic (BNP); brain natriuretic peptide; thyroid stimulating hormone; electrocardiograph (EKG); and digoxin level.

In some embodiments, the de-centralized healthcare system further comprises a hygiene system. In some embodiments, the hygiene system comprises a hygienist, wherein the hygienist enters the residence of the subject to perform hygiene duties. In some embodiments, the subject is incapable of performing hygiene duties. In some embodiments, the hygienist enters the residence of the subject daily, semi-weekly, weekly, biweekly, or monthly. In some embodiments, the hygiene duties comprise laundry, cleaning bed sheets, sanitizing, and disinfecting. In some embodiments, the hygiene system is independent of the de-centralized healthcare system and cooperates with the de-centralized healthcare system.

In some embodiments, the de-centralized healthcare system further comprises a food delivery system. In some embodiments, the subject is incapable of providing food. In some embodiments, the food delivery system comprises daily, semi-weekly, weekly, bi-weekly, or monthly delivery of a food supply. In some embodiments, the food supply comprises one or more of prepared meals, packaged foods, frozen foods, groceries, nutritional supplements, or foods comprising a nutritional regimen, wherein the nutritional regimen is provided by the de-centralized healthcare system. In some embodiments, the food delivery system is independent of the de-centralized healthcare system and cooperates with the de-centralized healthcare system.

In some embodiments, the invention contemplates a central station for providing healthcare to one or more subjects, the central station comprising: a) a computer system comprising a database comprising a subject, wherein the subject comprises a designated health condition and resides at a location remote from the central station, wherein the database comprises a record for the subject; b) a staff comprising a healthcare practitioner; c) a first communication system comprising audio/visual communication between the staff and the subject; and d) a second communication system comprising communication between the staff and a dispatch system comprising a healthcare provider, wherein the dispatch system dispatches the healthcare provider to the subject, wherein the subject is in need or want of healthcare. In some embodiments, each of the subjects independently resides at a private home, a nursing home, an apartment, a hotel, or an assisted-living facility. In some embodiments, the first communication system is effective to monitor the subject from the central station.

In some embodiments, the invention contemplates a method of providing healthcare to a subject, the method comprising: a) providing a central station for the management of a de-centralized healthcare system, wherein the central station comprises a healthcare practitioner; b) registering a subject in the de-centralized healthcare system, wherein the subject comprises a designated health condition; c) providing the subject with means for a self-administered healthcare procedure; and d) providing a communication system comprising communication between the subject and the central station, whereby the healthcare practitioner advises the subject on performing the self-administered therapy according to a care pathway protocol, wherein the subject resides at a location remote from the central station. In some embodiments, the subject resides at a private home, a nursing home, an apartment, a hotel, or an assisted-living facility. In some embodiments, the self-administered healthcare procedure comprises one or more of a medical regimen, an exercise regimen, a nutritional regimen, a mental aptitude regimen, a morale regimen, a hygiene regimen, a self-injection of an injectable material, a self-medication with a medication, a self-application of a topical material, a self-examination, a use of a device, a physical therapy, or a collection of a bodily fluid, a tissue or a discharge. In some embodiments, the topical material comprises a lotion, a salve, an ointment, a cream, an oil, an article of clothing, or a device worn on the body. In some embodiments, the injectable material, the medication, the topical material, or the device is provided by the de-centralized healthcare system. In some embodiments, the self-examination comprises the use of a kit provided by the de-centralized healthcare system. In some embodiments, the collection of a bodily fluid, a tissue or a discharge comprises the use of a kit provided by the de-centralized healthcare system. In some embodiments, the communication system comprising communication between the subject and the central station comprises written, audio, visual, monitoring, or audio/visual communication, or a combination thereof. In some embodiments, the communication system is effective to enable a diagnosis, an evaluation, or an assessment of the subject by the healthcare practitioner.

In some embodiments, the method further comprises providing at-home healthcare to the subject wherein the subject is in need or want of at-home healthcare. In some embodiments, the subject requests at-home healthcare. In some embodiments, a device alerts the central station to a need for at-home healthcare. In some embodiments, the at-home healthcare is a scheduled healthcare service, a routine healthcare service, or an emergency healthcare service. In some embodiments, the scheduled healthcare service or the routine healthcare service comprises an appointment, an examination of the subject, an examination of a device, a check-up, a follow-up, a visit, an adjustment, or a delivery or a pick-up of an item. In some embodiments, the item comprises a medication, a vitamin, a dietary supplement, an article of clothing, a device, a report, a questionnaire, a kit, or a sample, wherein the sample comprises a body fluid, a tissue, or a discharge. In some embodiments, the emergency healthcare service comprises a resuscitation, a defibrillation, an injection, a rescue from a sudden condition, or a transporting of the subject to a clinic or a hospital. In some embodiments, the sudden condition comprises a heart attack, a stroke, an instance of shock, a loss of blood pressure, a loss of consciousness, a fall, a wound, an adverse reaction to medication, an allergic reaction, or a failure of a device.

In some embodiments, the method further comprises: a) providing a dispatch system comprising a healthcare provider; b) dispatching the healthcare provider from the dispatch system to the subject, wherein the subject is in need or want of healthcare; and c) administering a healthcare service to the subject via the healthcare provider according to a care pathway protocol. In some embodiments, the healthcare provider is a physician, a clinician, a nurse, a paramedic, a therapist, a pharmacist, or a veterinarian. In some embodiments, the physician is a doctor of medicine, osteopathy, chiropractic, podiatry, optometry, or dentistry.

In some embodiments, the invention contemplates a method of providing at-home healthcare to an at-home subject, the method comprising: a) providing a central station for the management of a de-centralized healthcare system; b) providing a dispatch system comprising a healthcare provider; c) registering a subject in the de-centralized healthcare system, wherein the at-home subject comprises a designated health condition; d) providing a communication system comprising audio/visual communication between the subject and the central station, optionally whereby the subject requests healthcare; e) optionally providing one or more devices to the home of the at-home subject, wherein the one or more devices are effective to alert the central station to a need for at-home healthcare; f) dispatching the healthcare provider from the dispatch system to the subject, wherein the subject is in need or want of healthcare; and g) administering a healthcare service to the subject via the healthcare provider according to a care pathway protocol.

In some embodiments, the invention contemplates a method of selecting a subject for a de-centralized healthcare system, the method comprising: a) providing a subject comprising a condition; b) providing a list comprising a plurality of designated health conditions; c) diagnosing the condition of the subject to provide a diagnosed condition; d) comparing the diagnosed condition to the list comprising a plurality of designated health conditions; e) selecting a subject based on the results of step d) to provide a selected subject; and f) registering the selected subject in the de-centralized healthcare system to provide a registered subject.

In some embodiments, the invention contemplates a method of selecting a subject for a de-centralized healthcare system, the method comprising: a) providing a subject comprising a condition; b) diagnosing the condition of the subject to provide a diagnosed condition; c) performing a cost/benefit analysis for caring for the subject in a hospital system; d) performing a cost/benefit analysis for caring for the subject in the de-centralized healthcare system; e) comparing the results of steps c) and d); f) selecting a subject based on the results of step e) to provide a selected subject; and g) registering the selected subject in the de-centralized healthcare system to provide a registered subject.

In some embodiments, the invention contemplates a method of selecting a subject for end-of-life care in a de-centralized healthcare system, the method comprising: a) providing a subject, wherein the subject is at or near the end of life and comprises a quality of life; b) predicting the quality of life of the subject in a hospital system; c) predicting the quality of life of the subject in the de-centralized healthcare system; d) comparing the results of steps b) and c); e) selecting a subject based on the results of step d) to provide a selected subject; and f) registering the selected subject in the de-centralized healthcare system to provide a registered subject.

In some embodiments, the invention contemplates a de-centralized healthcare system for the treatment of a plurality of designated health conditions, wherein the designated health conditions include asthma/bronchitis, cellulitis, chronic heart failure (CHF), chronic obstructive pulmonary disease (COPD), deep venous thrombosis, diverticulitis, disequilibrium (vertigo), gastroenteritis/esophagitis, kidney and urinary tract infection, pneumonia, dehydration, viral illness, diversion diagnosis, post post-op central nervous system (CNS) procedures, post-op cardiac bypass, post-op orthopedic procedures, medical back problems, strokes, seizures, neoplasms and lymphoma related hospitalizations, chronic infections, major cardiac surgery, coronary bypass surgery, permanent cardiac pacemaker, vascular procedures, post-myocardial infarction (MI), gastrointestinal (GI) procedures (small and large bowel), pulmonary embolism, respiratory failure with vent support, specific end-stage disease states, ventilator care, external feeding care, New York Heart Association (NYHA) class IV heart failure, severe/symptomatic valvular heart disease in individuals who are poor surgical candidates, Global Initiative for Chronic Obstructive Lung Disease (GOLD) classification stage IV chronic obstructive pulmonary disease (COPD)/respiratory failure, malignant disease, failure to thrive of senescence, acquired immunodeficiency syndrome (AIDS), cirrhosis in individuals who are not candidates for transplantation, and amyotrophic lateral sclerosis.

In some embodiments, the invention contemplates a de-centralized healthcare system for the treatment of a plurality of designated health conditions, wherein the designated health conditions include: heart transplant or implant of heart assist system; craniotomy and endovascular intracranial procedures; extracranial procedures; multiple sclerosis and cerebellar ataxia; intracranial hemorrhage or cerebral infarction; transient ischemia; nonspecific cerebrovascular disorders; seizures; headaches; disequilibrium (vertigo); major chest procedures; other respiratory system operating room procedures; pulmonary embolism; respiratory infections and inflammations; respiratory neoplasms; pulmonary edema and respiratory failure; chronic obstructive pulmonary disease; simple pneumonia and pleurisy; bronchitis and asthma; respiratory signs and symptoms; respiratory system diagnosis with ventilator support; deep venous thrombosis, chronic heart failure; cardiac valve and other major cardiothoracic procedures; cardiac defibrillator implant with cardiac catheterization with acute myocardial infarction/heart failure/shock; cardiac defibrillator implant without cardiac catheterization with major coronary bypass with percutaneous transluminal coronary angioplasty; major cardiovascular procedures or thoracic aorta; permanent cardiac pacemaker implant; percutaneous cardiovascular procedure with drug-eluting stent or other circulatory system operating room procedures; other vascular procedures; cardiac pacemaker revision except device replacement; other circulatory system operating room procedures; acute myocardial infarction; circulatory disorders except acute myocardial infarction, with cardiac catheterization; heart failure and shock; deep vein thrombophlebitis; dehydration; diversion diagnosis; end-stage disease states; external feeding; New York Heart Association class IV heart failure; severe/symptomatic valvular heart disease in individuals who are poor surgical candidates; Global Initiative for Chronic Obstructive Lung Disease classification stage IV COPD/respiratory failure; malignant disease; failure to thrive of senescence; acquired immunodeficiency syndrome (AIDS); cirrhosis in individuals who are not candidates for transplantation; amyotrophic lateral sclerosis; peripheral vascular disorders; atherosclerosis; hypertension; cardiac arrhythmia and conduction disorders; syncope and collapse; chest pain; other circulatory system diagnoses; diverticulitis; post-operative central nervous system procedures; strokes; neoplasms and lymphoma-related hospitalizations; chronic infections; stomach, esophageal and duodenal procedures; major small and large bowel procedures with other digestive system operating room procedures; peritoneal adhesiolysis; hernia procedures except inguinal and femoral; other digestive system operating room procedures; major gastrointestinal disorders and peritoneal infection; digestive malignancy; gastrointestinal hemorrhage; complicated peptic ulcer; gastrointestinal obstruction; esophagitis, gastroenterological and miscellaneous digestive disorders; other digestive system diagnoses; laparoscopic cholecystectomy without common bile duct exploration; cirrhosis and alcoholic hepatitis; malignancy of hepatobiliary system or pancreas; disorders of pancreas except malignancy; disorders of liver except malignancy, cirrhosis, alcoholic hepatitis; disorders of the biliary tract; spinal fusion except cervical; wound debridement and skin graft except hand, for musculoskeletal-connective tissue; revision of hip or knee replacement; major joint replacement or reattachment of lower extremity; hip and femur procedures except major joint; knee procedures with past diagnosis of infection; back and neck procedures except spinal fusion or disctectomy; lower extremety and humer procedures except hip, foot, femur; other musculoskeletal system and connective tissue operating room procedures; fractures of hip and pelvis; osteomyelitis; pathological fractures and musculoskeletal and connective tissue fractures, sprains, strains and dislocations except femur, hip, pelvis and skin graft and/or debridement for skin ulceration or cellulitis; connective tissue disorders; medical back problems; tendonitis, myositis and bursitis; fracture, sprain, strain and dislocation except femur, hip, and pelvis; skin graft and/or debridement for skin ulcer or cellulitis; other skin, subcutaneous tissue and breast procedures; skin ulcers; cellulitis; skin grafts and wound debridement for endocrine, nutrition, and metabolic disorders; other endocrine, nutrition and metabolic operating room procedures; diabetes; nutritional and miscellaneous metabolic disorders; endocrine disorders; major bladder procedures; kidney and ureter procedures for neoplasm; transurethral procedures; other kidney and urinary tract procedures; renal failure; kidney and urinary tract infections; urinary stones without electronic shockwave lithotripsy; other kidney and urinary tract diagnoses; major male pelvic procedures, conization, laparoscopy and tubal interruption; other female reproductive system operating procedures; lymphoma and non-acute leukemia; infectious and parasitic diseases with operating room procedure; postoperative or post-traumatic infections within operating rooms; fever; viral illness; septicemia or severe sepsis without mechanical ventilation for at least 96 hours; poisoning and toxic effects of drugs; complications of treatment; signs and symptoms; HIV with major related conditions; extensive operating room procedure unrelated to principal disorder; and non-extensive operating room procedure unrelated to principal disorder.

In some embodiments, the invention contemplates a central station for the treatment of a plurality of designated health conditions, wherein the designated health conditions include asthma/bronchitis, cellulitis, chronic heart failure (CHF), chronic obstructive pulmonary disease (COPD), deep venous thrombosis, diverticulitis, disequilibrium (vertigo), gastroenteritis/esophagitis, kidney and urinary tract infection, pneumonia, dehydration, viral illness, diversion diagnosis, post post-op central nervous system (CNS) procedures, post-op cardiac bypass, post-op orthopedic procedures, medical back problems, strokes, seizures, neoplasms and lymphoma related hospitalizations, chronic infections, major cardiac surgery, coronary bypass surgery, permanent cardiac pacemaker, vascular procedures, post-myocardial infarction (MI), gastrointestinal (GI) procedures (small and large bowel), pulmonary embolism, respiratory failure with vent support, specific end-stage disease states, ventilator care, external feeding care, New York Heart Association (NYHA) class IV heart failure, severe/symptomatic valvular heart disease in individuals who are poor surgical candidates, Global Initiative for Chronic Obstructive Lung Disease (GOLD) classification stage IV chronic obstructive pulmonary disease (COPD)/respiratory failure, malignant disease, failure to thrive of senescence, acquired immunodeficiency syndrome (AIDS), cirrhosis in individuals who are not candidates for transplantation, and amyotrophic lateral sclerosis.

In some embodiments, the invention contemplates a central station for the treatment of a plurality of designated health conditions, wherein the designated health conditions include: heart transplant or implant of heart assist system; craniotomy and endovascular intracranial procedures; extracranial procedures; multiple sclerosis and cerebellar ataxia; intracranial hemorrhage or cerebral infarction; transient ischemia; nonspecific cerebrovascular disorders; seizures; headaches; disequilibrium (vertigo); major chest procedures; other respiratory system operating room procedures; pulmonary embolism; respiratory infections and inflammations; respiratory neoplasms; pulmonary edema and respiratory failure; chronic obstructive pulmonary disease; simple pneumonia and pleurisy; bronchitis and asthma; respiratory signs and symptoms; respiratory system diagnosis with ventilator support; deep venous thrombosis, chronic heart failure; cardiac valve and other major cardiothoracic procedures; cardiac defibrillator implant with cardiac catheterization with acute myocardial infarction/heart failure/shock; cardiac defibrillator implant without cardiac catheterization with major coronary bypass with percutaneous transluminal coronary angioplasty; major cardiovascular procedures or thoracic aorta; permanent cardiac pacemaker implant; percutaneous cardiovascular procedure with drug-eluting stent or other circulatory system operating room procedures; other vascular procedures; cardiac pacemaker revision except device replacement; other circulatory system operating room procedures; acute myocardial infarction; circulatory disorders except acute myocardial infarction, with cardiac catheterization; heart failure and shock; deep vein thrombophlebitis; dehydration; diversion diagnosis; end-stage disease states; external feeding; New York Heart Association class IV heart failure; severe/symptomatic valvular heart disease in individuals who are poor surgical candidates; Global Initiative for Chronic Obstructive Lung Disease classification stage IV COPD/respiratory failure; malignant disease; failure to thrive of senescence; acquired immunodeficiency syndrome (AIDS); cirrhosis in individuals who are not candidates for transplantation; amyotrophic lateral sclerosis; peripheral vascular disorders; atherosclerosis; hypertension; cardiac arrhythmia and conduction disorders; syncope and collapse; chest pain; other circulatory system diagnoses; diverticulitis; post-operative central nervous system procedures; strokes; neoplasms and lymphoma-related hospitalizations; chronic infections; stomach, esophageal and duodenal procedures; major small and large bowel procedures with other digestive system operating room procedures; peritoneal adhesiolysis; hernia procedures except inguinal and femoral; other digestive system operating room procedures; major gastrointestinal disorders and peritoneal infection; digestive malignancy; gastrointestinal hemorrhage; complicated peptic ulcer; gastrointestinal obstruction; esophagitis, gastroenterological and miscellaneous digestive disorders; other digestive system diagnoses; laparoscopic cholecystectomy without common bile duct exploration; cirrhosis and alcoholic hepatitis; malignancy of hepatobiliary system or pancreas; disorders of pancreas except malignancy; disorders of liver except malignancy, cirrhosis, alcoholic hepatitis; disorders of the biliary tract; spinal fusion except cervical; wound debridement and skin graft except hand, for musculoskeletal-connective tissue; revision of hip or knee replacement; major joint replacement or reattachment of lower extremity; hip and femur procedures except major joint; knee procedures with past diagnosis of infection; back and neck procedures except spinal fusion or disctectomy; lower extremety and humer procedures except hip, foot, femur; other musculoskeletal system and connective tissue operating room procedures; fractures of hip and pelvis; osteomyelitis; pathological fractures and musculoskeletal and connective tissue fractures, sprains, strains and dislocations except femur, hip, pelvis and skin graft and/or debridement for skin ulceration or cellulitis; connective tissue disorders; medical back problems; tendonitis, myositis and bursitis; fracture, sprain, strain and dislocation except femur, hip, and pelvis; skin graft and/or debridement for skin ulcer or cellulitis; other skin, subcutaneous tissue and breast procedures; skin ulcers; cellulitis; skin grafts and wound debridement for endocrine, nutrition, and metabolic disorders; other endocrine, nutrition and metabolic operating room procedures; diabetes; nutritional and miscellaneous metabolic disorders; endocrine disorders; major bladder procedures; kidney and ureter procedures for neoplasm; transurethral procedures; other kidney and urinary tract procedures; renal failure; kidney and urinary tract infections; urinary stones without electronic shockwave lithotripsy; other kidney and urinary tract diagnoses; major male pelvic procedures, conization, laparoscopy and tubal interruption; other female reproductive system operating procedures; lymphoma and non-acute leukemia; infectious and parasitic diseases with operating room procedure; postoperative or post-traumatic infections within operating rooms; fever; viral illness; septicemia or severe sepsis without mechanical ventilation for at least 96 hours; poisoning and toxic effects of drugs; complications of treatment; signs and symptoms; HIV with major related conditions; extensive operating room procedure unrelated to principal disorder; and non-extensive operating room procedure unrelated to principal disorder.

In some embodiments, the invention contemplates a method for treating a plurality of designated health conditions, wherein the designated health conditions include asthma/bronchitis, cellulitis, chronic heart failure (CHF), chronic obstructive pulmonary disease (COPD), deep venous thrombosis, diverticulitis, disequilibrium (vertigo), gastroenteritis/esophagitis, kidney and urinary tract infection, pneumonia, dehydration, viral illness, diversion diagnosis, post post-op central nervous system (CNS) procedures, post-op cardiac bypass, post-op orthopedic procedures, medical back problems, strokes, seizures, neoplasms and lymphoma related hospitalizations, chronic infections, major cardiac surgery, coronary bypass surgery, permanent cardiac pacemaker, vascular procedures, post-myocardial infarction (MI), gastrointestinal (GI) procedures (small and large bowel), pulmonary embolism, respiratory failure with vent support, specific end-stage disease states, ventilator care, external feeding care, New York Heart Association (NYHA) class IV heart failure, severe/symptomatic valvular heart disease in individuals who are poor surgical candidates, Global Initiative for Chronic Obstructive Lung Disease (GOLD) classification stage IV chronic obstructive pulmonary disease (COPD)/respiratory failure, malignant disease, failure to thrive of senescence, acquired immunodeficiency syndrome (AIDS), cirrhosis in individuals who are not candidates for transplantation, and amyotrophic lateral sclerosis.

In some embodiments, the invention contemplates a method for treating a plurality of designated health conditions, wherein the designated health conditions include: heart transplant or implant of heart assist system; craniotomy and endovascular intracranial procedures; extracranial procedures; multiple sclerosis and cerebellar ataxia; intracranial hemorrhage or cerebral infarction; transient ischemia; nonspecific cerebrovascular disorders; seizures; headaches; disequilibrium (vertigo); major chest procedures; other respiratory system operating room procedures; pulmonary embolism; respiratory infections and inflammations; respiratory neoplasms; pulmonary edema and respiratory failure; chronic obstructive pulmonary disease; simple pneumonia and pleurisy; bronchitis and asthma; respiratory signs and symptoms; respiratory system diagnosis with ventilator support; deep venous thrombosis, chronic heart failure; cardiac valve and other major cardiothoracic procedures; cardiac defibrillator implant with cardiac catheterization with acute myocardial infarction/heart failure/shock; cardiac defibrillator implant without cardiac catheterization with major coronary bypass with percutaneous transluminal coronary angioplasty; major cardiovascular procedures or thoracic aorta; permanent cardiac pacemaker implant; percutaneous cardiovascular procedure with drug-eluting stent or other circulatory system operating room procedures; other vascular procedures; cardiac pacemaker revision except device replacement; other circulatory system operating room procedures; acute myocardial infarction; circulatory disorders except acute myocardial infarction, with cardiac catheterization; heart failure and shock; deep vein thrombophlebitis; dehydration; diversion diagnosis; end-stage disease states; external feeding; New York Heart Association class IV heart failure; severe/symptomatic valvular heart disease in individuals who are poor surgical candidates; Global Initiative for Chronic Obstructive Lung Disease classification stage IV COPD/respiratory failure; malignant disease; failure to thrive of senescence; acquired immunodeficiency syndrome (AIDS); cirrhosis in individuals who are not candidates for transplantation; amyotrophic lateral sclerosis; peripheral vascular disorders; atherosclerosis; hypertension; cardiac arrhythmia and conduction disorders; syncope and collapse; chest pain; other circulatory system diagnoses; diverticulitis; post-operative central nervous system procedures; strokes; neoplasms and lymphoma-related hospitalizations; chronic infections; stomach, esophageal and duodenal procedures; major small and large bowel procedures with other digestive system operating room procedures; peritoneal adhesiolysis; hernia procedures except inguinal and femoral; other digestive system operating room procedures; major gastrointestinal disorders and peritoneal infection; digestive malignancy; gastrointestinal hemorrhage; complicated peptic ulcer; gastrointestinal obstruction; esophagitis, gastroenterological and miscellaneous digestive disorders; other digestive system diagnoses; laparoscopic cholecystectomy without common bile duct exploration; cirrhosis and alcoholic hepatitis; malignancy of hepatobiliary system or pancreas; disorders of pancreas except malignancy; disorders of liver except malignancy, cirrhosis, alcoholic hepatitis; disorders of the biliary tract; spinal fusion except cervical; wound debridement and skin graft except hand, for musculoskeletal-connective tissue; revision of hip or knee replacement; major joint replacement or reattachment of lower extremity; hip and femur procedures except major joint; knee procedures with past diagnosis of infection; back and neck procedures except spinal fusion or disctectomy; lower extremety and humer procedures except hip, foot, femur; other musculoskeletal system and connective tissue operating room procedures; fractures of hip and pelvis; osteomyelitis; pathological fractures and musculoskeletal and connective tissue fractures, sprains, strains and dislocations except femur, hip, pelvis and skin graft and/or debridement for skin ulceration or cellulitis; connective tissue disorders; medical back problems; tendonitis, myositis and bursitis; fracture, sprain, strain and dislocation except femur, hip, and pelvis; skin graft and/or debridement for skin ulcer or cellulitis; other skin, subcutaneous tissue and breast procedures; skin ulcers; cellulitis; skin grafts and wound debridement for endocrine, nutrition, and metabolic disorders; other endocrine, nutrition and metabolic operating room procedures; diabetes; nutritional and miscellaneous metabolic disorders; endocrine disorders; major bladder procedures; kidney and ureter procedures for neoplasm; transurethral procedures; other kidney and urinary tract procedures; renal failure; kidney and urinary tract infections; urinary stones without electronic shockwave lithotripsy; other kidney and urinary tract diagnoses; major male pelvic procedures, conization, laparoscopy and tubal interruption; other female reproductive system operating procedures; lymphoma and non-acute leukemia; infectious and parasitic diseases with operating room procedure; postoperative or post-traumatic infections within operating rooms; fever; viral illness; septicemia or severe sepsis without mechanical ventilation for at least 96 hours; poisoning and toxic effects of drugs; complications of treatment; signs and symptoms; HIV with major related conditions; extensive operating room procedure unrelated to principal disorder; and non-extensive operating room procedure unrelated to principal disorder.

In some embodiments, the invention contemplates a de-centralized healthcare system, wherein the de-centralized healthcare system comprises affiliation, collaboration, communication, or cooperation with at least one conventional healthcare facility. In some embodiments, the affiliation, collaboration, communication, or cooperation with at least one conventional healthcare facility provides subjects and records to the de-centralized healthcare system.

In some embodiments, the invention contemplates a de-centralized healthcare system comprising one or more care pathway protocols, wherein the care pathway protocol comprises:

a) Subject Qualification and Admission, comprising: 1) Stability Criteria, comprising: Subject status, Psychological and Social safety status; and 2) Assessments, comprising: Subject's Health, Home Assessments, and Nutritional Needs;

b) Orders, Treatments, and Activities, comprising: medications, subject activities, nutritional supplementation, procedures, durable medical equipment, and optionally interventions effective to improve the health of the subject;

c) Monitoring, comprising: biometric and telecommunication equipment to monitor the subject;

d) Education and Care Coordination/Integration, comprising: Instructive exercises and literature; and

e) Advanced Medical Directive, comprising: discussions regarding one or both of chronic care management and advanced care planning

In some embodiments, the invention contemplates a method comprising the use of one or more care pathway protocols, wherein the care pathway protocol comprises:

a) Subject Qualification and Admission, comprising: 1) Stability Criteria, comprising: Subject status, Psychological and Social safety status; and 2) Assessments, comprising: Subject's Health, Home Assessments, and Nutritional Needs;

b) Orders, Treatments, and Activities, comprising: medications, subject activities, nutritional supplementation, procedures, durable medical equipment, and optionally interventions effective to improve the health of the subject;

c) Monitoring, comprising: biometric and telecommunication equipment to monitor the subject;

d) Education and Care Coordination/Integration, comprising: Instructive exercises and literature; and

e) Advanced Medical Directive, comprising: discussions regarding one or both of chronic care management and advanced care planning

In some embodiments, the invention contemplates a care pathway protocol, comprising:

a) Subject Qualification and Admission, comprising: 1) Stability Criteria, comprising: Subject status, Psychological and Social safety status; and 2) Assessments, comprising: Subject's Health, Home Assessments, and Nutritional Needs;

b) Orders, Treatments, and Activities, comprising: medications, subject activities, nutritional supplementation, procedures, durable medical equipment, and optionally interventions effective to improve the health of the subject;

c) Monitoring, comprising: biometric and telecommunication equipment to monitor the subject;

d) Education and Care Coordination/Integration, comprising: Instructive exercises and literature; and

e) Advanced Medical Directive, comprising: discussions regarding one or both of chronic care management and advanced care planning

In some embodiments, the invention contemplates a method of providing acute healthcare to a subject, the method comprising:

a) Qualifying and admitting a subject, comprising: 1) Evaluating Stability Criteria, comprising: Subject status, Psychological and Social safety status; and 2) Assessing subject health, home, and nutritional needs;

b) Issuing Orders, Treatments, and Activities, comprising: medications, subject activities, nutritional supplementation, procedures, durable medical equipment, and optionally interventions effective to improve the health of the subject;

c) Monitoring, comprising: biometric and telecommunication equipment to monitor the subject;

d) Educating a subject on Care Coordination/Integration, the educating comprising: Instructive exercises and literature; and optionally

e) Providing an advanced medical directive, comprising: discussions regarding one or both of chronic care management and advanced care planning.

In some embodiments, the invention contemplates a de-centralized healthcare system for providing healthcare to one or more at-home subjects, the system comprising: a) a central station comprising a healthcare practitioner; b) a subject, wherein the subject resides at a location remote from the central station and is registered with the de-centralized healthcare system, and wherein the subject comprises a designated health condition; c) a communication system comprising audio/visual communication between the subject and the healthcare practitioner, whereby the healthcare practitioner provides a first healthcare service to the subject according to a first care pathway protocol; d) a dispatch system comprising a healthcare provider, wherein the dispatch system dispatches the healthcare provider to the subject, wherein the subject is in need or want of healthcare, and further wherein the healthcare provider provides a second healthcare service to the subject according to a second care pathway protocol; and e) a plurality of care pathway protocols, wherein each care pathway protocol comprises a protocol for providing healthcare for a selected designated health condition to a subject in need or want thereof.

In some embodiments, the invention contemplates a de-centralized healthcare system comprising: a) a central station staffed by at least one healthcare practitioner; b) a plurality of care pathway protocols, wherein each care pathway protocol comprises a protocol for providing healthcare for a selected designated health condition to a subject in need or want thereof; and c) a communication system for communication between the subject and the healthcare practitioner, wherein the subject resides at a location remote from the central station and is registered with the de-centralized healthcare system.

In some embodiments, the central station is effective to monitor the health, status, and location of the subject.

In some embodiments, the central station further comprises a record-keeping system, a computing system, and a library of reference materials, wherein the record-keeping system comprises a record for each subject registered with the de-centralized healthcare system.

In some embodiments, the designated health condition comprises one or more of asthma/bronchitis, cellulitis, chronic heart failure (CHF), chronic obstructive pulmonary disease (COPD), deep venous thrombosis, diverticulitis, disequilibrium (vertigo), gastroenteritis/esophagitis, kidney and urinary tract infection, pneumonia, dehydration, viral illness, diversion diagnosis, post-op central nervous system (CNS) procedures, post-op cardiac bypass, post-op orthopedic procedures, medical back problems, strokes, seizures, neoplasms and lymphoma related hospitalizations, chronic infections, major cardiac surgery, coronary bypass surgery, permanent cardiac pacemaker, vascular procedures, post-myocardial infarction (MI), gastrointestinal (GI) procedures (small and large bowel), pulmonary embolism, respiratory failure with vent support, specific end-stage disease states, ventilator care, external feeding care, New York Heart Association (NYHA) class IV heart failure, severe/symptomatic valvular heart disease in individuals who are poor surgical candidates, Global Initiative for Chronic Obstructive Lung Disease (GOLD) classification stage IV chronic obstructive pulmonary disease (COPD)/respiratory failure, malignant disease, failure to thrive of senescence, acquired immunodeficiency syndrome (AIDS), cirrhosis in individuals who are not candidates for transplantation, and amyotrophic lateral sclerosis.

In some embodiments, the designated health condition comprises one or more of heart transplant or implant of heart assist system; craniotomy and endovascular intracranial procedures; extracranial procedures; multiple sclerosis and cerebellar ataxia; intracranial hemorrhage or cerebral infarction; transient ischemia; nonspecific cerebrovascular disorders; seizures; headaches; disequilibrium (vertigo); major chest procedures; other respiratory system operating room procedures; pulmonary embolism; respiratory infections and inflammations; respiratory neoplasms; pulmonary edema and respiratory failure; chronic obstructive pulmonary disease; simple pneumonia and pleurisy; bronchitis and asthma; respiratory signs and symptoms; respiratory system diagnosis with ventilator support; deep venous thrombosis, chronic heart failure; cardiac valve and other major cardiothoracic procedures; cardiac defibrillator implant with cardiac catheterization with acute myocardial infarction/heart failure/shock; cardiac defibrillator implant without cardiac catheterization with major coronary bypass with percutaneous transluminal coronary angioplasty; major cardiovascular procedures or thoracic aorta; permanent cardiac pacemaker implant; percutaneous cardiovascular procedure with drug-eluting stent or other circulatory system operating room procedures; other vascular procedures; cardiac pacemaker revision except device replacement; other circulatory system operating room procedures; acute myocardial infarction; circulatory disorders except acute myocardial infarction, with cardiac catheterization; heart failure and shock; deep vein thrombophlebitis; dehydration; diversion diagnosis; end-stage disease states; external feeding; New York Heart Association class IV heart failure; severe/symptomatic valvular heart disease in individuals who are poor surgical candidates; Global Initiative for Chronic Obstructive Lung Disease classification stage IV COPD/respiratory failure; malignant disease; failure to thrive of senescence; acquired immunodeficiency syndrome (AIDS); cirrhosis in individuals who are not candidates for transplantation; amyotrophic lateral sclerosis; peripheral vascular disorders; atherosclerosis; hypertension; cardiac arrhythmia and conduction disorders; syncope and collapse; chest pain; other circulatory system diagnoses; diverticulitis; post-operative central nervous system procedures; strokes; neoplasms and lymphoma-related hospitalizations; chronic infections; stomach, esophageal and duodenal procedures; major small and large bowel procedures with other digestive system operating room procedures; peritoneal adhesiolysis; hernia procedures except inguinal and femoral; other digestive system operating room procedures; major gastrointestinal disorders and peritoneal infection; digestive malignancy; gastrointestinal hemorrhage; complicated peptic ulcer; gastrointestinal obstruction; esophagitis, gastroenterological and miscellaneous digestive disorders; other digestive system diagnoses; laparoscopic cholecystectomy without common bile duct exploration; cirrhosis and alcoholic hepatitis; malignancy of hepatobiliary system or pancreas; disorders of pancreas except malignancy; disorders of liver except malignancy, cirrhosis, alcoholic hepatitis; disorders of the biliary tract; spinal fusion except cervical; wound debridement and skin graft except hand, for musculoskeletal-connective tissue; revision of hip or knee replacement; major joint replacement or reattachment of lower extremity; hip and femur procedures except major joint; knee procedures with past diagnosis of infection; back and neck procedures except spinal fusion or disctectomy; lower extremety and humer procedures except hip, foot, femur; other musculoskeletal system and connective tissue operating room procedures; fractures of hip and pelvis; osteomyelitis; pathological fractures and musculoskeletal and connective tissue fractures, sprains, strains and dislocations except femur, hip, pelvis and skin graft and/or debridement for skin ulceration or cellulitis; connective tissue disorders; medical back problems; tendonitis, myositis and bursitis; fracture, sprain, strain and dislocation except femur, hip, and pelvis; skin graft and/or debridement for skin ulcer or cellulitis; other skin, subcutaneous tissue and breast procedures; skin ulcers; cellulitis; skin grafts and wound debridement for endocrine, nutrition, and metabolic disorders; other endocrine, nutrition and metabolic operating room procedures; diabetes; nutritional and miscellaneous metabolic disorders; endocrine disorders; major bladder procedures; kidney and ureter procedures for neoplasm; transurethral procedures; other kidney and urinary tract procedures; renal failure; kidney and urinary tract infections; urinary stones without electronic shockwave lithotripsy; other kidney and urinary tract diagnoses; major male pelvic procedures, conization, laparoscopy and tubal interruption; other female reproductive system operating procedures; lymphoma and non-acute leukemia; infectious and parasitic diseases with operating room procedure; postoperative or post-traumatic infections within operating rooms; fever; viral illness; septicemia or severe sepsis without mechanical ventilation for at least 96 hours; poisoning and toxic effects of drugs; complications of treatment; signs and symptoms; HIV with major related conditions; extensive operating room procedure unrelated to principal disorder; and non-extensive operating room procedure unrelated to principal disorder.

In some embodiments, the de-centralized healthcare system affiliates, collaborates, communicates, or cooperates with at least one conventional healthcare facility.

In some embodiments, each care pathway protocol comprises:

a) subject qualification and admission, comprising: 1) stability criteria, comprising: subject status, psychological and social safety status; and 2) assessments, comprising: subject's health, home assessments, and nutritional needs;

b) orders, treatments, and activities, comprising: medications, subject activities, nutritional supplementation, procedures, durable medical equipment, and optionally interventions effective to improve the health of the subject;

c) monitoring, comprising: biometric and telecommunication equipment to monitor the subject;

d) education and care coordination/integration, comprising: instructive exercises and literature; and

e) advanced medical directive, comprising: discussions regarding one or both of chronic care management and advanced care planning

In some embodiments, the central station is staffed by at least two healthcare practitioners.

In some embodiments, the healthcare practitioner is a doctor of medicine, a doctor of osteopathy, or a nurse practitioner.

In some embodiments, the healthcare practitioner is available by appointment, on-call, or for emergencies.

In some embodiments, the healthcare practitioner provides a healthcare service to the subject.

In some embodiments, the subject comprises a designated health condition.

In some embodiments, the healthcare practitioner is a veterinarian. In some embodiments, the subject is the owner of an animal. In some embodiments, the animal is in need of healthcare or the owner is in of want of healthcare for the animal. In some embodiments, the animal is a pet, a guardian, a livestock, a service animal, a show animal, a working animal, a racing animal, or a breeding animal. In some embodiments, the animal is a mammal, a reptile, a bird, an amphibian, or an aquatic animal. In some embodiments, the animal is a mammal.

In some embodiments, the subject resides at a private home, a nursing home, an apartment, a hotel, or an assisted-living facility.

In some embodiments, the subject does not leave home to receive healthcare service.

In some embodiments, the subject requests a healthcare service via the communication system.

In some embodiments, the healthcare service comprises an advice, an instruction, or a recommendation.

In some embodiments, the subject is educated in self-administration of a healthcare procedure, wherein the healthcare procedure comprises a self-injection of an injectable material, a self-medication with a medication, a self-application of a topical material, a self-examination, a use of a device, a physical therapy, or a collection of a bodily fluid, a tissue or a discharge.

In some embodiments, the healthcare procedure comprises the use of a kit provided by the de-centralized healthcare system.

In some embodiments, the subject is at or near the end-of-life.

In some embodiments, each care pathway protocol of the plurality of care pathway protocols is effective to provide healthcare to the subject for one or more designated health conditions.

In some embodiments, each designated health condition has at least one care pathway protocol.

In some embodiments, each of the care pathway protocols is used independently.

In some embodiments, the healthcare practitioner performs at least one of the care pathway protocols.

In some embodiments, more than one care pathway protocols are used synergistically.

In some embodiments, the healthcare practitioner diagnoses, evaluates, or assesses the subject via the communication system.

In some embodiments, the communication system comprises one or more of written, audio, visual, monitoring, and audio/visual communication.

In some embodiments, the healthcare practitioner provides a healthcare service to the subject via the communication system.

In some embodiments, the healthcare service comprises one or more of an advice, an instruction, a recommendation, an inquiry, or a prescription.

In some embodiments, the de-centralized healthcare system further comprises a dispatch system.

In some embodiments, the dispatch system is located at the same facility as the central station.

In some embodiments, dispatch system is located at a different facility than the central station.

In some embodiments, the dispatch system is staffed by at least one healthcare provider.

In some embodiments, the healthcare provider is a physician, a clinician, a nurse, a paramedic, a therapist, a pharmacist, or a veterinarian.

In some embodiments, the physician is a doctor of medicine, osteopathy, chiropractic, podiatry, optometry, or dentistry.

In some embodiments, the dispatch system dispatches the healthcare provider to the subject, wherein the subject is in need or want of healthcare.

In some embodiments, the healthcare provider provides a healthcare service to the subject according to a care pathway protocol.

In some embodiments, the subject requests that the dispatch system dispatch the healthcare provider to the subject.

In some embodiments, the subject requests an emergency healthcare service.

In some embodiments, the subject had not previously requested that the dispatch system dispatch the healthcare provider to the subject.

In some embodiments, a monitoring device alerts the central station to a need for the emergency healthcare service.

In some embodiments, the de-centralized healthcare system further comprises a plurality of kits, wherein each kit independently comprises materials effective for self-administration of a healthcare service.

In some embodiments, the de-centralized healthcare system further comprises a laboratory.

In some embodiments, the laboratory comprises facilities for performing an analysis of a sample provided by the subject.

In some embodiments, the de-centralized healthcare system further comprises a hygiene system. In some embodiments, the hygiene system comprises an at least one hygienist, wherein the at least one hygienist enters the residence of the subject to perform one or more hygiene duties. In some embodiments, the subject is incapable of performing the hygiene duties. In some embodiments, the hygienist enters the residence of the subject daily, semi-weekly, weekly, biweekly, or monthly. In some embodiments, the hygiene duties comprise laundry, cleaning bed sheets, sanitizing, and disinfecting.

In some embodiments, the de-centralized healthcare system further comprises a food delivery system. In some embodiments, the subject is incapable of providing food for the subject. In some embodiments, the food delivery system provides daily, semi-weekly, weekly, bi-weekly, or monthly delivery of a food supply. In some embodiments, the food supply comprises one or more of prepared meals, packaged foods, frozen foods, groceries, nutritional supplements, and foods comprising a nutritional regimen.

In some embodiments, the invention contemplates a method of providing healthcare, the method comprising: a) providing a central station for the management of a de-centralized healthcare system, wherein the central station is staffed by at least one healthcare practitioner; b) registering a subject in the de-centralized healthcare system, wherein the subject comprises a designated health condition; c) providing the subject with means for a self-administered healthcare procedure; and d) providing a communication system comprising communication between the subject and the central station, whereby the healthcare practitioner advises the subject on performing the self-administered healthcare procedure according to a care pathway protocol, wherein the subject resides at a location remote from the central station.

In some embodiments, the designated health condition comprises one or more of asthma/bronchitis, cellulitis, chronic heart failure (CHF), chronic obstructive pulmonary disease (COPD), deep venous thrombosis, diverticulitis, disequilibrium (vertigo), gastroenteritis/esophagitis, kidney and urinary tract infection, pneumonia, dehydration, viral illness, diversion diagnosis, post-op central nervous system (CNS) procedures, post-op cardiac bypass, post-op orthopedic procedures, medical back problems, strokes, seizures, neoplasms and lymphoma related hospitalizations, chronic infections, major cardiac surgery, coronary bypass surgery, permanent cardiac pacemaker, vascular procedures, post-myocardial infarction (MI), gastrointestinal (GI) procedures (small and large bowel), pulmonary embolism, respiratory failure with vent support, specific end-stage disease states, ventilator care, external feeding care, New York Heart Association (NYHA) class IV heart failure, severe/symptomatic valvular heart disease in individuals who are poor surgical candidates, Global Initiative for Chronic Obstructive Lung Disease (GOLD) classification stage IV chronic obstructive pulmonary disease (COPD)/respiratory failure, malignant disease, failure to thrive of senescence, acquired immunodeficiency syndrome (AIDS), cirrhosis in individuals who are not candidates for transplantation, and amyotrophic lateral sclerosis.

In some embodiments, the designated health condition comprises one or more of: heart transplant or implant of heart assist system; craniotomy and endovascular intracranial procedures; extracranial procedures; multiple sclerosis and cerebellar ataxia; intracranial hemorrhage or cerebral infarction; transient ischemia; nonspecific cerebrovascular disorders; seizures; headaches; disequilibrium (vertigo); major chest procedures; other respiratory system operating room procedures; pulmonary embolism; respiratory infections and inflammations; respiratory neoplasms; pulmonary edema and respiratory failure; chronic obstructive pulmonary disease; simple pneumonia and pleurisy; bronchitis and asthma; respiratory signs and symptoms; respiratory system diagnosis with ventilator support; deep venous thrombosis, chronic heart failure; cardiac valve and other major cardiothoracic procedures; cardiac defibrillator implant with cardiac catheterization with acute myocardial infarction/heart failure/shock; cardiac defibrillator implant without cardiac catheterization with major coronary bypass with percutaneous transluminal coronary angioplasty; major cardiovascular procedures or thoracic aorta; permanent cardiac pacemaker implant; percutaneous cardiovascular procedure with drug-eluting stent or other circulatory system operating room procedures; other vascular procedures; cardiac pacemaker revision except device replacement; other circulatory system operating room procedures; acute myocardial infarction; circulatory disorders except acute myocardial infarction, with cardiac catheterization; heart failure and shock; deep vein thrombophlebitis; dehydration; diversion diagnosis; end-stage disease states; external feeding; New York Heart Association class IV heart failure; severe/symptomatic valvular heart disease in individuals who are poor surgical candidates; Global Initiative for Chronic Obstructive Lung Disease classification stage IV COPD/respiratory failure; malignant disease; failure to thrive of senescence; acquired immunodeficiency syndrome (AIDS); cirrhosis in individuals who are not candidates for transplantation; amyotrophic lateral sclerosis; peripheral vascular disorders; atherosclerosis; hypertension; cardiac arrhythmia and conduction disorders; syncope and collapse; chest pain; other circulatory system diagnoses; diverticulitis; post-operative central nervous system procedures; strokes; neoplasms and lymphoma-related hospitalizations; chronic infections; stomach, esophageal and duodenal procedures; major small and large bowel procedures with other digestive system operating room procedures; peritoneal adhesiolysis; hernia procedures except inguinal and femoral; other digestive system operating room procedures; major gastrointestinal disorders and peritoneal infection; digestive malignancy; gastrointestinal hemorrhage; complicated peptic ulcer; gastrointestinal obstruction; esophagitis, gastroenterological and miscellaneous digestive disorders; other digestive system diagnoses; laparoscopic cholecystectomy without common bile duct exploration; cirrhosis and alcoholic hepatitis; malignancy of hepatobiliary system or pancreas; disorders of pancreas except malignancy; disorders of liver except malignancy, cirrhosis, alcoholic hepatitis; disorders of the biliary tract; spinal fusion except cervical; wound debridement and skin graft except hand, for musculoskeletal-connective tissue; revision of hip or knee replacement; major joint replacement or reattachment of lower extremity; hip and femur procedures except major joint; knee procedures with past diagnosis of infection; back and neck procedures except spinal fusion or disctectomy; lower extremety and humer procedures except hip, foot, femur; other musculoskeletal system and connective tissue operating room procedures; fractures of hip and pelvis; osteomyelitis; pathological fractures and musculoskeletal and connective tissue fractures, sprains, strains and dislocations except femur, hip, pelvis and skin graft and/or debridement for skin ulceration or cellulitis; connective tissue disorders; medical back problems; tendonitis, myositis and bursitis; fracture, sprain, strain and dislocation except femur, hip, and pelvis; skin graft and/or debridement for skin ulcer or cellulitis; other skin, subcutaneous tissue and breast procedures; skin ulcers; cellulitis; skin grafts and wound debridement for endocrine, nutrition, and metabolic disorders; other endocrine, nutrition and metabolic operating room procedures; diabetes; nutritional and miscellaneous metabolic disorders; endocrine disorders; major bladder procedures; kidney and ureter procedures for neoplasm; transurethral procedures; other kidney and urinary tract procedures; renal failure; kidney and urinary tract infections; urinary stones without electronic shockwave lithotripsy; other kidney and urinary tract diagnoses; major male pelvic procedures, conization, laparoscopy and tubal interruption; other female reproductive system operating procedures; lymphoma and non-acute leukemia; infectious and parasitic diseases with operating room procedure; postoperative or post-traumatic infections within operating rooms; fever; viral illness; septicemia or severe sepsis without mechanical ventilation for at least 96 hours; poisoning and toxic effects of drugs; complications of treatment; signs and symptoms; HIV with major related conditions; extensive operating room procedure unrelated to principal disorder; and non-extensive operating room procedure unrelated to principal disorder.

In some embodiments, the subject resides at a private home, a nursing home, an apartment, a hotel, or an assisted-living facility.

In some embodiments, the self-administered healthcare procedure comprises one or more of a medical regimen, an exercise regimen, a nutritional regimen, a mental aptitude regimen, a morale regimen, a hygiene regimen, a self-injection of an injectable material, a self-medication with a medication, a self-application of a topical material, a self-examination, a use of a device, a physical therapy, or a collection of a bodily fluid, a tissue or a discharge.

In some embodiments, the healthcare comprises one or more of a medical regimen, an exercise regimen, a nutritional regimen, a mental aptitude regimen, a morale regimen, a hygiene regimen, a self-injection of an injectable material, a self-medication with a medication, a self-application of a topical material, a self-examination, a use of a device, a physical therapy, or a collection of a bodily fluid, a tissue or a discharge.

In some embodiments, the communication system comprises written, audio, visual, monitoring, or audio/visual communication, or a combination thereof.

In some embodiments, the healthcare practitioner diagnoses, evaluates, or assesses the subject via the communication system.

In some embodiments, the care pathway protocol comprises:

a) subject qualification and admission, comprising: 1) stability criteria, comprising: subject status, psychological and social safety status; and 2) assessments, comprising: subject's health, home assessments, and nutritional needs;

b) orders, treatments, and activities, comprising: medications, subject activities, nutritional supplementation, procedures, durable medical equipment, and optionally interventions effective to improve the health of the subject;

c) monitoring, comprising: biometric and telecommunication equipment to monitor the subject;

d) education and care coordination/integration, comprising: instructive exercises and literature; and

e) advanced medical directive, comprising: discussions regarding one or both of chronic care management and advanced care planning

In some embodiments, the method further comprises providing at-home healthcare to the subject, wherein the subject is in need or want of at-home healthcare.

In some embodiments, the subject requests at-home healthcare.

In some embodiments, a device alerts the central station to a need for at-home healthcare.

In some embodiments, the method further comprises: a) providing a dispatch system staffed by at least one healthcare provider; b) dispatching the healthcare provider from the dispatch system to the subject, wherein the subject is in need or want of healthcare; and c) administering a healthcare service to the subject via the healthcare provider according to a care pathway protocol.

In some embodiments, the de-centralized healthcare system further comprises: a dispatch system staffed by at least one healthcare provider, wherein the dispatch system dispatches the healthcare provider to the subject, whereby the healthcare provider provides healthcare to the subject according to a care pathway protocol.

In some embodiments, the healthcare provider is a physician, a clinician, a nurse, a paramedic, a therapist, a pharmacist, or a veterinarian.

In some embodiments, the physician is a doctor of medicine, osteopathy, chiropractic, podiatry, optometry, or dentistry.

In some embodiments, the invention contemplates a method of providing at-home healthcare, the method comprising: a) providing a central station for the management of a de-centralized healthcare system; b) providing a dispatch system staffed by at least one healthcare provider; c) registering a subject in the de-centralized healthcare system, wherein the subject comprises a designated health condition; d) providing a communication system comprising audio/visual communication between the subject and the central station, optionally whereby the subject requests healthcare; e) optionally providing one or more devices and durable medical equipment to the home of the subject, wherein the devices are effective to alert the central station to a need for at-home healthcare; f) dispatching the healthcare provider from the dispatch system to the subject, wherein the subject is in need or want of healthcare; and g) administering a healthcare service to the subject via the healthcare provider according to a care pathway protocol.

In some embodiments, the invention contemplates a method of selecting a subject for a de-centralized healthcare system, the method comprising: a) providing a subject comprising a condition; b) providing a list comprising a plurality of designated health conditions; c) diagnosing the condition of the subject to provide a diagnosed condition; d) comparing the diagnosed condition to the list comprising a plurality of designated health conditions; e) selecting a subject based on the results of step d) to provide a selected subject; and f) registering the selected subject in the de-centralized healthcare system to provide a registered subject, wherein the de-centralized healthcare system comprises: 1) a central station staffed by at least one healthcare practitioner; 2) a plurality of care pathway protocols, wherein each care pathway protocol comprises a protocol for providing healthcare for a selected designated health condition; and 3) a communication system for communication between the registered subject and the healthcare practitioner, wherein the registered subject resides at a location remote from the central station.

In some embodiments, the designated health condition comprises one or more of asthma/bronchitis, cellulitis, chronic heart failure (CHF), chronic obstructive pulmonary disease (COPD), deep venous thrombosis, diverticulitis, disequilibrium (vertigo), gastroenteritis/esophagitis, kidney and urinary tract infection, pneumonia, dehydration, viral illness, diversion diagnosis, post-op central nervous system (CNS) procedures, post-op cardiac bypass, post-op orthopedic procedures, medical back problems, strokes, seizures, neoplasms and lymphoma related hospitalizations, chronic infections, major cardiac surgery, coronary bypass surgery, permanent cardiac pacemaker, vascular procedures, post-myocardial infarction (MI), gastrointestinal (GI) procedures (small and large bowel), pulmonary embolism, respiratory failure with vent support, specific end-stage disease states, ventilator care, external feeding care, New York Heart Association (NYHA) class IV heart failure, severe/symptomatic valvular heart disease in individuals who are poor surgical candidates, Global Initiative for Chronic Obstructive Lung Disease (GOLD) classification stage IV chronic obstructive pulmonary disease (COPD)/respiratory failure, malignant disease, failure to thrive of senescence, acquired immunodeficiency syndrome (AIDS), cirrhosis in individuals who are not candidates for transplantation, and amyotrophic lateral sclerosis.

In some embodiments, the designated health condition comprises one or more of: heart transplant or implant of heart assist system; craniotomy and endovascular intracranial procedures; extracranial procedures; multiple sclerosis and cerebellar ataxia; intracranial hemorrhage or cerebral infarction; transient ischemia; nonspecific cerebrovascular disorders; seizures; headaches; disequilibrium (vertigo); major chest procedures; other respiratory system operating room procedures; pulmonary embolism; respiratory infections and inflammations; respiratory neoplasms; pulmonary edema and respiratory failure; chronic obstructive pulmonary disease; simple pneumonia and pleurisy; bronchitis and asthma; respiratory signs and symptoms; respiratory system diagnosis with ventilator support; deep venous thrombosis, chronic heart failure; cardiac valve and other major cardiothoracic procedures; cardiac defibrillator implant with cardiac catheterization with acute myocardial infarction/heart failure/shock; cardiac defibrillator implant without cardiac catheterization with major coronary bypass with percutaneous transluminal coronary angioplasty; major cardiovascular procedures or thoracic aorta; permanent cardiac pacemaker implant; percutaneous cardiovascular procedure with drug-eluting stent or other circulatory system operating room procedures; other vascular procedures; cardiac pacemaker revision except device replacement; other circulatory system operating room procedures; acute myocardial infarction; circulatory disorders except acute myocardial infarction, with cardiac catheterization; heart failure and shock; deep vein thrombophlebitis; dehydration; diversion diagnosis; end-stage disease states; external feeding; New York Heart Association class IV heart failure; severe/symptomatic valvular heart disease in individuals who are poor surgical candidates; Global Initiative for Chronic Obstructive Lung Disease classification stage IV COPD/respiratory failure; malignant disease; failure to thrive of senescence; acquired immunodeficiency syndrome (AIDS); cirrhosis in individuals who are not candidates for transplantation; amyotrophic lateral sclerosis; peripheral vascular disorders; atherosclerosis; hypertension; cardiac arrhythmia and conduction disorders; syncope and collapse; chest pain; other circulatory system diagnoses; diverticulitis; post-operative central nervous system procedures; strokes; neoplasms and lymphoma-related hospitalizations; chronic infections; stomach, esophageal and duodenal procedures; major small and large bowel procedures with other digestive system operating room procedures; peritoneal adhesiolysis; hernia procedures except inguinal and femoral; other digestive system operating room procedures; major gastrointestinal disorders and peritoneal infection; digestive malignancy; gastrointestinal hemorrhage; complicated peptic ulcer; gastrointestinal obstruction; esophagitis, gastroenterological and miscellaneous digestive disorders; other digestive system diagnoses; laparoscopic cholecystectomy without common bile duct exploration; cirrhosis and alcoholic hepatitis; malignancy of hepatobiliary system or pancreas; disorders of pancreas except malignancy; disorders of liver except malignancy, cirrhosis, alcoholic hepatitis; disorders of the biliary tract; spinal fusion except cervical; wound debridement and skin graft except hand, for musculoskeletal-connective tissue; revision of hip or knee replacement; major joint replacement or reattachment of lower extremity; hip and femur procedures except major joint; knee procedures with past diagnosis of infection; back and neck procedures except spinal fusion or disctectomy; lower extremety and humer procedures except hip, foot, femur; other musculoskeletal system and connective tissue operating room procedures; fractures of hip and pelvis; osteomyelitis; pathological fractures and musculoskeletal and connective tissue fractures, sprains, strains and dislocations except femur, hip, pelvis and skin graft and/or debridement for skin ulceration or cellulitis; connective tissue disorders; medical back problems; tendonitis, myositis and bursitis; fracture, sprain, strain and dislocation except femur, hip, and pelvis; skin graft and/or debridement for skin ulcer or cellulitis; other skin, subcutaneous tissue and breast procedures; skin ulcers; cellulitis; skin grafts and wound debridement for endocrine, nutrition, and metabolic disorders; other endocrine, nutrition and metabolic operating room procedures; diabetes; nutritional and miscellaneous metabolic disorders; endocrine disorders; major bladder procedures; kidney and ureter procedures for neoplasm; transurethral procedures; other kidney and urinary tract procedures; renal failure; kidney and urinary tract infections; urinary stones without electronic shockwave lithotripsy; other kidney and urinary tract diagnoses; major male pelvic procedures, conization, laparoscopy and tubal interruption; other female reproductive system operating procedures; lymphoma and non-acute leukemia; infectious and parasitic diseases with operating room procedure; postoperative or post-traumatic infections within operating rooms; fever; viral illness; septicemia or severe sepsis without mechanical ventilation for at least 96 hours; poisoning and toxic effects of drugs; complications of treatment; signs and symptoms; HIV with major related conditions; extensive operating room procedure unrelated to principal disorder; and non-extensive operating room procedure unrelated to principal disorder.

In some embodiments, the invention contemplates a method of selecting a subject for a de-centralized healthcare system, the method comprising: a) providing a subject comprising a condition; b) diagnosing the condition of the subject to provide a diagnosed condition; c) performing a cost/benefit analysis for caring for the subject in a hospital system; d) performing a cost/benefit analysis for caring for the subject in the de-centralized healthcare system; e) comparing the results of steps c) and d); f) selecting a subject based on the results of step e) to provide a selected subject; and g) registering the selected subject in the de-centralized healthcare system to provide a registered subject, wherein, the de-centralized healthcare system comprises: 1) a central station staffed by at least one healthcare practitioner; 2) a plurality of care pathway protocols, wherein each care pathway protocol comprises a protocol for providing healthcare for a selected designated health condition; and 3) a communication system for communication between the registered subject and the healthcare practitioner, wherein the registered subject resides at a location remote from the central station.

In some embodiments, the invention contemplates a care pathway protocol, comprising:

a) subject qualification and admission, comprising: 1) stability criteria, comprising: subject status, psychological and social safety status; and 2) assessments, comprising: subject's health, home assessments, and nutritional needs;

b) orders, treatments, and activities, comprising: medications, subject activities, nutritional supplementation, procedures, durable medical equipment, and optionally interventions effective to improve the health of the subject;

c) monitoring, comprising: biometric and telecommunication equipment to monitor the subject;

d) education and care coordination/integration, comprising: instructive exercises and literature; and

e) advanced medical directive, comprising: discussions regarding one or both of chronic care management and advanced care planning

In some embodiments, the invention contemplates a method of providing acute healthcare to a subject, the method comprising:

a) qualifying and admitting a subject, comprising: 1) evaluating the subject's stability criteria, comprising the subject's status, and psychological and social safety status; and 2) assessing the subject's health, home, and nutritional needs;

b) issuing orders, treatments, and activities for the subject, comprising: medications, subject activities, nutritional supplementation, procedures, durable medical equipment, and optionally interventions effective to improve the health of the subject;

c) monitoring the subject, comprising: providing biometric and telecommunication equipment to monitor the subject and monitoring the subject with the biometric and telecommunication equipment;

d) educating the subject on care coordination/integration, the educating comprising: instructive exercises and literature; and

e) providing an advanced medical directive, comprising: discussing one or both of chronic care management and advanced care planning

In some embodiments, the invention contemplates a de-centralized healthcare system consisting essentially of: a) a central station staffed by at least one healthcare practitioner; b) a plurality of care pathway protocols, wherein each care pathway protocol comprises a protocol for providing healthcare for a selected designated health condition to a subject in need or want thereof; and c) a communication system for communication between the subject and the healthcare practitioner, wherein the subject resides at a location remote from the central station and is registered with the de-centralized healthcare system.

In some embodiments, the invention contemplates a de-centralized healthcare system consisting essentially of: a) a central station staffed by at least one healthcare practitioner; b) a plurality of care pathway protocols, wherein each care pathway protocol comprises a protocol for providing healthcare for a selected designated health condition to a subject in need or want thereof; c) a communication system for communication between the subject and the healthcare practitioner; and d) a dispatch system, wherein the subject resides at a location remote from the central station and is registered with the de-centralized healthcare system.

In some embodiments, the invention contemplates a de-centralized healthcare system consisting essentially of: a) a central station staffed by at least one healthcare practitioner; b) a plurality of care pathway protocols, wherein each care pathway protocol comprises a protocol for providing healthcare for a selected designated health condition to a subject in need or want thereof; c) a communication system for communication between the subject and the healthcare practitioner; d) a dispatch system; and e) a laboratory, wherein the subject resides at a location remote from the central station and is registered with the de-centralized healthcare system.

In some embodiments, the invention contemplates a de-centralized healthcare system consisting essentially of: a) a central station staffed by at least one healthcare practitioner; b) a plurality of care pathway protocols, wherein each care pathway protocol comprises a protocol for providing healthcare for a selected designated health condition to a subject in need or want thereof; c) a communication system for communication between the subject and the healthcare practitioner; d) a dispatch system; e) a laboratory; and f) a plurality of kits, wherein the subject resides at a location remote from the central station and is registered with the de-centralized healthcare system.

In some embodiments, the invention contemplates a de-centralized healthcare system consisting essentially of: a) a central station staffed by at least one healthcare practitioner; b) a plurality of care pathway protocols, wherein each care pathway protocol comprises a protocol for providing healthcare for a selected designated health condition to a subject in need or want thereof; c) a communication system for communication between the subject and the healthcare practitioner; d) a dispatch system; e) a laboratory; f) a hygiene system; and g) a food delivery system, wherein the subject resides at a location remote from the central station and is registered with the de-centralized healthcare system.

In some embodiments, the invention contemplates a de-centralized healthcare system consisting essentially of: a) a central station staffed by at least one healthcare practitioner; b) a plurality of care pathway protocols, wherein each care pathway protocol comprises a protocol for providing healthcare for a selected designated health condition to a subject in need or want thereof; c) a communication system for communication between the subject and the healthcare practitioner; d) a dispatch system; e) a laboratory; f) a plurality of kits; g) a hygiene system; and h) a food delivery system, wherein the subject resides at a location remote from the central station and is registered with the de-centralized healthcare system.

In some embodiments, the invention contemplates a use of a de-centralized healthcare system to provide healthcare, the de-centralized healthcare system comprising: a) a central station staffed by at least one healthcare practitioner; b) a plurality of care pathway protocols, wherein each care pathway protocol comprises a protocol for providing healthcare for a selected designated health condition to a subject in need or want thereof; and c) a communication system for communication between the subject and the healthcare practitioner, wherein the subject resides at a location remote from the central station and is registered with the de-centralized healthcare system.

In some embodiments, the invention contemplates a de-centralized healthcare system for use in the treatment of a designated health condition, the de-centralized healthcare system comprising: a) a central station staffed by at least one healthcare practitioner; b) a plurality of care pathway protocols, wherein each care pathway protocol comprises a protocol for providing healthcare for a selected designated health condition to a subject in need or want thereof; and c) a communication system for communication between the subject and the healthcare practitioner, wherein the subject resides at a location remote from the central station and is registered with the de-centralized healthcare system.

In some embodiments, the invention contemplates a de-centralized healthcare system for use in the treatment of a designated health condition, the de-centralized healthcare system comprising: a) a central station staffed by at least one healthcare practitioner; b) a plurality of care pathway protocols, wherein each care pathway protocol comprises a protocol for providing healthcare for a selected designated health condition to a subject in need or want thereof; and c) a communication system for communication between the subject and the healthcare practitioner, wherein the subject resides at a location remote from the central station and is registered with the de-centralized healthcare system, and wherein the designated health condition is asthma/bronchitis, cellulitis, chronic heart failure (CHF), chronic obstructive pulmonary disease (COPD), deep venous thrombosis, diverticulitis, disequilibrium (vertigo), gastroenteritis/esophagitis, kidney and urinary tract infection, pneumonia, dehydration, viral illness, diversion diagnosis, post-op central nervous system (CNS) procedures, post-op cardiac bypass, post-op orthopedic procedures, medical back problems, strokes, seizures, neoplasms and lymphoma related hospitalizations, chronic infections, major cardiac surgery, coronary bypass surgery, permanent cardiac pacemaker, vascular procedures, post-myocardial infarction (MI), gastrointestinal (GI) procedures (small and large bowel), pulmonary embolism, respiratory failure with vent support, specific end-stage disease states, ventilator care, external feeding care, New York Heart Association (NYHA) class IV heart failure, severe/symptomatic valvular heart disease in individuals who are poor surgical candidates, Global Initiative for Chronic Obstructive Lung Disease (GOLD) classification stage IV chronic obstructive pulmonary disease (COPD)/respiratory failure, malignant disease, failure to thrive of senescence, acquired immunodeficiency syndrome (AIDS), cirrhosis in individuals who are not candidates for transplantation, or amyotrophic lateral sclerosis.

In some embodiments, the invention contemplates a de-centralized healthcare system for use in the treatment of a designated health condition, the de-centralized healthcare system comprising: a) a central station staffed by at least one healthcare practitioner; b) a plurality of care pathway protocols, wherein each care pathway protocol comprises a protocol for providing healthcare for a selected designated health condition to a subject in need or want thereof; and c) a communication system for communication between the subject and the healthcare practitioner, wherein the subject resides at a location remote from the central station and is registered with the de-centralized healthcare system, and wherein the designated health condition is one of: heart transplant or implant of heart assist system; craniotomy and endovascular intracranial procedures; extracranial procedures; multiple sclerosis and cerebellar ataxia; intracranial hemorrhage or cerebral infarction; transient ischemia; nonspecific cerebrovascular disorders; seizures; headaches; disequilibrium (vertigo); major chest procedures; other respiratory system operating room procedures; pulmonary embolism; respiratory infections and inflammations; respiratory neoplasms; pulmonary edema and respiratory failure; chronic obstructive pulmonary disease; simple pneumonia and pleurisy; bronchitis and asthma; respiratory signs and symptoms; respiratory system diagnosis with ventilator support; deep venous thrombosis, chronic heart failure; cardiac valve and other major cardiothoracic procedures; cardiac defibrillator implant with cardiac catheterization with acute myocardial infarction/heart failure/shock; cardiac defibrillator implant without cardiac catheterization with major coronary bypass with percutaneous transluminal coronary angioplasty; major cardiovascular procedures or thoracic aorta; permanent cardiac pacemaker implant; percutaneous cardiovascular procedure with drug-eluting stent or other circulatory system operating room procedures; other vascular procedures; cardiac pacemaker revision except device replacement; other circulatory system operating room procedures; acute myocardial infarction; circulatory disorders except acute myocardial infarction, with cardiac catheterization; heart failure and shock; deep vein thrombophlebitis; dehydration; diversion diagnosis; end-stage disease states; external feeding; New York Heart Association class IV heart failure; severe/symptomatic valvular heart disease in individuals who are poor surgical candidates; Global Initiative for Chronic Obstructive Lung Disease classification stage IV COPD/respiratory failure; malignant disease; failure to thrive of senescence; acquired immunodeficiency syndrome (AIDS); cirrhosis in individuals who are not candidates for transplantation; amyotrophic lateral sclerosis; peripheral vascular disorders; atherosclerosis; hypertension; cardiac arrhythmia and conduction disorders; syncope and collapse; chest pain; other circulatory system diagnoses; diverticulitis; post-operative central nervous system procedures; strokes; neoplasms and lymphoma-related hospitalizations; chronic infections; stomach, esophageal and duodenal procedures; major small and large bowel procedures with other digestive system operating room procedures; peritoneal adhesiolysis; hernia procedures except inguinal and femoral; other digestive system operating room procedures; major gastrointestinal disorders and peritoneal infection; digestive malignancy; gastrointestinal hemorrhage; complicated peptic ulcer; gastrointestinal obstruction; esophagitis, gastroenterological and miscellaneous digestive disorders; other digestive system diagnoses; laparoscopic cholecystectomy without common bile duct exploration; cirrhosis and alcoholic hepatitis; malignancy of hepatobiliary system or pancreas; disorders of pancreas except malignancy; disorders of liver except malignancy, cirrhosis, alcoholic hepatitis; disorders of the biliary tract; spinal fusion except cervical; wound debridement and skin graft except hand, for musculoskeletal-connective tissue; revision of hip or knee replacement; major joint replacement or reattachment of lower extremity; hip and femur procedures except major joint; knee procedures with past diagnosis of infection; back and neck procedures except spinal fusion or disctectomy; lower extremety and humer procedures except hip, foot, femur; other musculoskeletal system and connective tissue operating room procedures; fractures of hip and pelvis; osteomyelitis; pathological fractures and musculoskeletal and connective tissue fractures, sprains, strains and dislocations except femur, hip, pelvis and skin graft and/or debridement for skin ulceration or cellulitis; connective tissue disorders; medical back problems; tendonitis, myositis and bursitis; fracture, sprain, strain and dislocation except femur, hip, and pelvis; skin graft and/or debridement for skin ulcer or cellulitis; other skin, subcutaneous tissue and breast procedures; skin ulcers; cellulitis; skin grafts and wound debridement for endocrine, nutrition, and metabolic disorders; other endocrine, nutrition and metabolic operating room procedures; diabetes; nutritional and miscellaneous metabolic disorders; endocrine disorders; major bladder procedures; kidney and ureter procedures for neoplasm; transurethral procedures; other kidney and urinary tract procedures; renal failure; kidney and urinary tract infections; urinary stones without electronic shockwave lithotripsy; other kidney and urinary tract diagnoses; major male pelvic procedures, conization, laparoscopy and tubal interruption; other female reproductive system operating procedures; lymphoma and non-acute leukemia; infectious and parasitic diseases with operating room procedure; postoperative or post-traumatic infections within operating rooms; fever; viral illness; septicemia or severe sepsis without mechanical ventilation for at least 96 hours; poisoning and toxic effects of drugs; complications of treatment; signs and symptoms; HIV with major related conditions; extensive operating room procedure unrelated to principal disorder; and non-extensive operating room procedure unrelated to principal disorder.

In some embodiments, the invention contemplates a use of a care pathway protocol to provide healthcare to a subject, the care pathway protocol comprising:

a) subject qualification and admission, comprising: 1) stability criteria, comprising: subject status, psychological and social safety status; and 2) assessments, comprising: subject's health, home assessments, and nutritional needs;

b) orders, treatments, and activities, comprising: medications, subject activities, nutritional supplementation, procedures, durable medical equipment, and optionally interventions effective to improve the health of the subject;

c) monitoring, comprising: biometric and telecommunication equipment to monitor the subject;

d) education and care coordination/integration, comprising: instructive exercises and literature; and

e) advanced medical directive, comprising: discussions regarding one or both of chronic care management and advanced care planning

In some embodiments, the invention contemplates a use of a care pathway protocol to provide healthcare to a subject, the care pathway protocol comprising:

a) subject qualification and admission, comprising: 1) stability criteria, comprising: subject status, psychological and social safety status; and 2) assessments, comprising: subject's health, home assessments, and nutritional needs;

b) orders, treatments, and activities, comprising: medications, subject activities, nutritional supplementation, procedures, durable medical equipment, and optionally interventions effective to improve the health of the subject;

c) monitoring, comprising: biometric and telecommunication equipment to monitor the subject;

d) education and care coordination/integration, comprising: instructive exercises and literature; and

e) advanced medical directive, comprising: discussions regarding one or both of chronic care management and advanced care planning,

wherein the designated health condition is asthma/bronchitis, cellulitis, chronic heart failure (CHF), chronic obstructive pulmonary disease (COPD), deep venous thrombosis, diverticulitis, disequilibrium (vertigo), gastroenteritis/esophagitis, kidney and urinary tract infection, pneumonia, dehydration, viral illness, diversion diagnosis, post-op central nervous system (CNS) procedures, post-op cardiac bypass, post-op orthopedic procedures, medical back problems, strokes, seizures, neoplasms and lymphoma related hospitalizations, chronic infections, major cardiac surgery, coronary bypass surgery, permanent cardiac pacemaker, vascular procedures, post-myocardial infarction (MI), gastrointestinal (GI) procedures (small and large bowel), pulmonary embolism, respiratory failure with vent support, specific end-stage disease states, ventilator care, external feeding care, New York Heart Association (NYHA) class IV heart failure, severe/symptomatic valvular heart disease in individuals who are poor surgical candidates, Global Initiative for Chronic Obstructive Lung Disease (GOLD) classification stage IV chronic obstructive pulmonary disease (COPD)/respiratory failure, malignant disease, failure to thrive of senescence, acquired immunodeficiency syndrome (AIDS), cirrhosis in individuals who are not candidates for transplantation, or amyotrophic lateral sclerosis.

In some embodiments, the invention contemplates a use of a care pathway protocol to provide healthcare to a subject, the care pathway protocol comprising:

a) subject qualification and admission, comprising: 1) stability criteria, comprising: subject status, psychological and social safety status; and 2) assessments, comprising: subject's health, home assessments, and nutritional needs;

b) orders, treatments, and activities, comprising: medications, subject activities, nutritional supplementation, procedures, durable medical equipment, and optionally interventions effective to improve the health of the subject;

c) monitoring, comprising: biometric and telecommunication equipment to monitor the subject;

d) education and care coordination/integration, comprising: instructive exercises and literature; and

e) advanced medical directive, comprising: discussions regarding one or both of chronic care management and advanced care planning,

wherein the designated health condition is one of: heart transplant or implant of heart assist system; craniotomy and endovascular intracranial procedures; extracranial procedures; multiple sclerosis and cerebellar ataxia; intracranial hemorrhage or cerebral infarction; transient ischemia; nonspecific cerebrovascular disorders; seizures; headaches; disequilibrium (vertigo); major chest procedures; other respiratory system operating room procedures; pulmonary embolism; respiratory infections and inflammations; respiratory neoplasms; pulmonary edema and respiratory failure; chronic obstructive pulmonary disease; simple pneumonia and pleurisy; bronchitis and asthma; respiratory signs and symptoms; respiratory system diagnosis with ventilator support; deep venous thrombosis, chronic heart failure; cardiac valve and other major cardiothoracic procedures; cardiac defibrillator implant with cardiac catheterization with acute myocardial infarction/heart failure/shock; cardiac defibrillator implant without cardiac catheterization with major coronary bypass with percutaneous transluminal coronary angioplasty; major cardiovascular procedures or thoracic aorta; permanent cardiac pacemaker implant; percutaneous cardiovascular procedure with drug-eluting stent or other circulatory system operating room procedures; other vascular procedures; cardiac pacemaker revision except device replacement; other circulatory system operating room procedures; acute myocardial infarction; circulatory disorders except acute myocardial infarction, with cardiac catheterization; heart failure and shock; deep vein thrombophlebitis; dehydration; diversion diagnosis; end-stage disease states; external feeding; New York Heart Association class IV heart failure; severe/symptomatic valvular heart disease in individuals who are poor surgical candidates; Global Initiative for Chronic Obstructive Lung Disease classification stage IV COPD/respiratory failure; malignant disease; failure to thrive of senescence; acquired immunodeficiency syndrome (AIDS); cirrhosis in individuals who are not candidates for transplantation; amyotrophic lateral sclerosis; peripheral vascular disorders; atherosclerosis; hypertension; cardiac arrhythmia and conduction disorders; syncope and collapse; chest pain; other circulatory system diagnoses; diverticulitis; post-operative central nervous system procedures; strokes; neoplasms and lymphoma-related hospitalizations; chronic infections; stomach, esophageal and duodenal procedures; major small and large bowel procedures with other digestive system operating room procedures; peritoneal adhesiolysis; hernia procedures except inguinal and femoral; other digestive system operating room procedures; major gastrointestinal disorders and peritoneal infection; digestive malignancy; gastrointestinal hemorrhage; complicated peptic ulcer; gastrointestinal obstruction; esophagitis, gastroenterological and miscellaneous digestive disorders; other digestive system diagnoses; laparoscopic cholecystectomy without common bile duct exploration; cirrhosis and alcoholic hepatitis; malignancy of hepatobiliary system or pancreas; disorders of pancreas except malignancy; disorders of liver except malignancy, cirrhosis, alcoholic hepatitis; disorders of the biliary tract; spinal fusion except cervical; wound debridement and skin graft except hand, for musculoskeletal-connective tissue; revision of hip or knee replacement; major joint replacement or reattachment of lower extremity; hip and femur procedures except major joint; knee procedures with past diagnosis of infection; back and neck procedures except spinal fusion or disctectomy; lower extremety and humer procedures except hip, foot, femur; other musculoskeletal system and connective tissue operating room procedures; fractures of hip and pelvis; osteomyelitis; pathological fractures and musculoskeletal and connective tissue fractures, sprains, strains and dislocations except femur, hip, pelvis and skin graft and/or debridement for skin ulceration or cellulitis; connective tissue disorders; medical back problems; tendonitis, myositis and bursitis; fracture, sprain, strain and dislocation except femur, hip, and pelvis; skin graft and/or debridement for skin ulcer or cellulitis; other skin, subcutaneous tissue and breast procedures; skin ulcers; cellulitis; skin grafts and wound debridement for endocrine, nutrition, and metabolic disorders; other endocrine, nutrition and metabolic operating room procedures; diabetes; nutritional and miscellaneous metabolic disorders; endocrine disorders; major bladder procedures; kidney and ureter procedures for neoplasm; transurethral procedures; other kidney and urinary tract procedures; renal failure; kidney and urinary tract infections; urinary stones without electronic shockwave lithotripsy; other kidney and urinary tract diagnoses; major male pelvic procedures, conization, laparoscopy and tubal interruption; other female reproductive system operating procedures; lymphoma and non-acute leukemia; infectious and parasitic diseases with operating room procedure; postoperative or post-traumatic infections within operating rooms; fever; viral illness; septicemia or severe sepsis without mechanical ventilation for at least 96 hours; poisoning and toxic effects of drugs; complications of treatment; signs and symptoms; HIV with major related conditions; extensive operating room procedure unrelated to principal disorder; and non-extensive operating room procedure unrelated to principal disorder.

In some embodiments, the invention contemplates a care pathway protocol for use in providing healthcare to a subject, the care pathway protocol comprising:

a) subject qualification and admission, comprising: 1) stability criteria, comprising: subject status, psychological and social safety status; and 2) assessments, comprising: subject's health, home assessments, and nutritional needs;

b) orders, treatments, and activities, comprising: medications, subject activities, nutritional supplementation, procedures, durable medical equipment, and optionally interventions effective to improve the health of the subject;

c) monitoring, comprising: biometric and telecommunication equipment to monitor the subject;

d) education and care coordination/integration, comprising: instructive exercises and literature; and

e) advanced medical directive, comprising: discussions regarding one or both of chronic care management and advanced care planning

In some embodiments, the invention contemplates a care pathway protocol for use in providing healthcare to a subject, the care pathway protocol comprising:

a) subject qualification and admission, comprising: 1) stability criteria, comprising: subject status, psychological and social safety status; and 2) assessments, comprising: subject's health, home assessments, and nutritional needs;

b) orders, treatments, and activities, comprising: medications, subject activities, nutritional supplementation, procedures, durable medical equipment, and optionally interventions effective to improve the health of the subject;

c) monitoring, comprising: biometric and telecommunication equipment to monitor the subject;

d) education and care coordination/integration, comprising: instructive exercises and literature; and

e) advanced medical directive, comprising: discussions regarding one or both of chronic care management and advanced care planning,

wherein the designated health condition is asthma/bronchitis, cellulitis, chronic heart failure (CHF), chronic obstructive pulmonary disease (COPD), deep venous thrombosis, diverticulitis, disequilibrium (vertigo), gastroenteritis/esophagitis, kidney and urinary tract infection, pneumonia, dehydration, viral illness, diversion diagnosis, post-op central nervous system (CNS) procedures, post-op cardiac bypass, post-op orthopedic procedures, medical back problems, strokes, seizures, neoplasms and lymphoma related hospitalizations, chronic infections, major cardiac surgery, coronary bypass surgery, permanent cardiac pacemaker, vascular procedures, post-myocardial infarction (MI), gastrointestinal (GI) procedures (small and large bowel), pulmonary embolism, respiratory failure with vent support, specific end-stage disease states, ventilator care, external feeding care, New York Heart Association (NYHA) class IV heart failure, severe/symptomatic valvular heart disease in individuals who are poor surgical candidates, Global Initiative for Chronic Obstructive Lung Disease (GOLD) classification stage IV chronic obstructive pulmonary disease (COPD)/respiratory failure, malignant disease, failure to thrive of senescence, acquired immunodeficiency syndrome (AIDS), cirrhosis in individuals who are not candidates for transplantation, or amyotrophic lateral sclerosis.

In some embodiments, the invention contemplates a care pathway protocol for use in providing healthcare to a subject, the care pathway protocol comprising:

a) subject qualification and admission, comprising: 1) stability criteria, comprising: subject status, psychological and social safety status; and 2) assessments, comprising: subject's health, home assessments, and nutritional needs;

b) orders, treatments, and activities, comprising: medications, subject activities, nutritional supplementation, procedures, durable medical equipment, and optionally interventions effective to improve the health of the subject;

c) monitoring, comprising: biometric and telecommunication equipment to monitor the subject;

d) education and care coordination/integration, comprising: instructive exercises and literature; and

e) advanced medical directive, comprising: discussions regarding one or both of chronic care management and advanced care planning,

wherein the designated health condition is one of: heart transplant or implant of heart assist system; craniotomy and endovascular intracranial procedures; extracranial procedures; multiple sclerosis and cerebellar ataxia; intracranial hemorrhage or cerebral infarction; transient ischemia; nonspecific cerebrovascular disorders; seizures; headaches; disequilibrium (vertigo); major chest procedures; other respiratory system operating room procedures; pulmonary embolism; respiratory infections and inflammations; respiratory neoplasms; pulmonary edema and respiratory failure; chronic obstructive pulmonary disease; simple pneumonia and pleurisy; bronchitis and asthma; respiratory signs and symptoms; respiratory system diagnosis with ventilator support; deep venous thrombosis, chronic heart failure; cardiac valve and other major cardiothoracic procedures; cardiac defibrillator implant with cardiac catheterization with acute myocardial infarction/heart failure/shock; cardiac defibrillator implant without cardiac catheterization with major coronary bypass with percutaneous transluminal coronary angioplasty; major cardiovascular procedures or thoracic aorta; permanent cardiac pacemaker implant; percutaneous cardiovascular procedure with drug-eluting stent or other circulatory system operating room procedures; other vascular procedures; cardiac pacemaker revision except device replacement; other circulatory system operating room procedures; acute myocardial infarction; circulatory disorders except acute myocardial infarction, with cardiac catheterization; heart failure and shock; deep vein thrombophlebitis; dehydration; diversion diagnosis; end-stage disease states; external feeding; New York Heart Association class IV heart failure; severe/symptomatic valvular heart disease in individuals who are poor surgical candidates; Global Initiative for Chronic Obstructive Lung Disease classification stage IV COPD/respiratory failure; malignant disease; failure to thrive of senescence; acquired immunodeficiency syndrome (AIDS); cirrhosis in individuals who are not candidates for transplantation; amyotrophic lateral sclerosis; peripheral vascular disorders; atherosclerosis; hypertension; cardiac arrhythmia and conduction disorders; syncope and collapse; chest pain; other circulatory system diagnoses; diverticulitis; post-operative central nervous system procedures; strokes; neoplasms and lymphoma-related hospitalizations; chronic infections; stomach, esophageal and duodenal procedures; major small and large bowel procedures with other digestive system operating room procedures; peritoneal adhesiolysis; hernia procedures except inguinal and femoral; other digestive system operating room procedures; major gastrointestinal disorders and peritoneal infection; digestive malignancy; gastrointestinal hemorrhage; complicated peptic ulcer; gastrointestinal obstruction; esophagitis, gastroenterological and miscellaneous digestive disorders; other digestive system diagnoses; laparoscopic cholecystectomy without common bile duct exploration; cirrhosis and alcoholic hepatitis; malignancy of hepatobiliary system or pancreas; disorders of pancreas except malignancy; disorders of liver except malignancy, cirrhosis, alcoholic hepatitis; disorders of the biliary tract; spinal fusion except cervical; wound debridement and skin graft except hand, for musculoskeletal-connective tissue; revision of hip or knee replacement; major joint replacement or reattachment of lower extremity; hip and femur procedures except major joint; knee procedures with past diagnosis of infection; back and neck procedures except spinal fusion or disctectomy; lower extremety and humer procedures except hip, foot, femur; other musculoskeletal system and connective tissue operating room procedures; fractures of hip and pelvis; osteomyelitis; pathological fractures and musculoskeletal and connective tissue fractures, sprains, strains and dislocations except femur, hip, pelvis and skin graft and/or debridement for skin ulceration or cellulitis; connective tissue disorders; medical back problems; tendonitis, myositis and bursitis; fracture, sprain, strain and dislocation except femur, hip, and pelvis; skin graft and/or debridement for skin ulcer or cellulitis; other skin, subcutaneous tissue and breast procedures; skin ulcers; cellulitis; skin grafts and wound debridement for endocrine, nutrition, and metabolic disorders; other endocrine, nutrition and metabolic operating room procedures; diabetes; nutritional and miscellaneous metabolic disorders; endocrine disorders; major bladder procedures; kidney and ureter procedures for neoplasm; transurethral procedures; other kidney and urinary tract procedures; renal failure; kidney and urinary tract infections; urinary stones without electronic shockwave lithotripsy; other kidney and urinary tract diagnoses; major male pelvic procedures, conization, laparoscopy and tubal interruption; other female reproductive system operating procedures; lymphoma and non-acute leukemia; infectious and parasitic diseases with operating room procedure; postoperative or post-traumatic infections within operating rooms; fever; viral illness; septicemia or severe sepsis without mechanical ventilation for at least 96 hours; poisoning and toxic effects of drugs; complications of treatment; signs and symptoms; HIV with major related conditions; extensive operating room procedure unrelated to principal disorder; and non-extensive operating room procedure unrelated to principal disorder.

### Example 1

### A General Care Pathway Protocol

A subject is provided healthcare by the following protocol.

### Designated Health Condition:

### I. Subject Qualification and Admission.

### A. Stability Criteria.

1. Subject status: To be determined by physician.
2. Psychological and Social Safety Status: appropriate; cooperative; anxious; agitated; or depressed.

### B. Assessments.

1. Subject's Health: To be determined by physician (subject dependent).
2. Home Assessments: To be determined upon inspection (subject dependent).
3. Nutritional Needs: To be determined by physician (subject dependent).

### II. Orders, Treatments, and Activities.

A. Medications: To be determined by physician.

B. Subject Activities: To be determined by physician.

C. Nutritional Supplementation: To be determined by physician.

D. Procedures: To be determined by physician.

E. Durable Medical Equipment: adjustable beds, bed wedges, oxygen concentrator, wheelchair, walker, cane, bedpan, spacers, thermometer, pedal cycle, stretch band, pill boxes, handrails, shower seat, nebulizer, infusion equipment, IV delivery devices, and/or portable oxygen unit.

F. Interventions: To be determined by physician.

### III. Monitoring.

A. Biometric Equipment: glucose meter, pulse oximeter, weight scale, blood pressure cuff, EKG device, inclinometer, stethoscope, motion detectors, bed sensor, ADL monitor, biometric skin sensor, opthalmoscope, otoscope, ultrasound/echocardiogram device, impedance monitor, and/or temperature-reading device.

B. Telecommunication Equipment: two-way audio/video device, wireless hub, personal emergency response unit, and/or computer.

### IV. Education and Care Coordination/Integration.

A. Instructive Exercises: To be determined by physician.

B. Literature: To be determined by physician.

### V. Advanced Medical Directive.

A. Chronic Care Management: Physician discusses with subject if appropriate.

B. Advanced Care Planning: Physician discusses with subject if appropriate.

The Designated Health Condition describes the indication that the care pathway protocol is effective to treat.

Subject Qualification and Admission describe the subject's eligibility to become registered with the remote healthcare system. These factors can vary among subjects, and are evaluated by a physician.

Stability criteria describes the subject's eligibility to register and ability to remain receptive and responsive to healthcare services and healthcare practitioners and providers after registration with the de-centralized healthcare system. Subject status describes the health, marital, and residential status of the subject. Psychological and Social safety status describes the ability of the subject to interact constructively with healthcare practitioners and providers, to focus on health improvement, and to avoid self-destructive behaviors. Assessments include evaluations of the subject's health, home, and nutritional needs. The subject's health is evaluated to determine the suitability of the subject for registration with the remote healthcare system, and to determine the types, frequency, and aggressiveness of the healthcare services that the subject requires. The subject's home is inspected for suitability and adaptivity to protocols of the remote healthcare system and for safety considerations including: visibility, maneuverability (for example, absence of encumbering obstacles or steep stairways), proper utilities, handicap accessibility if required, and proximity to neighbors. The subject's nutritional needs are investigated to determine if the subject would benefit from a nutritional regimen, for example, a low-salt diet, a high fluid intake, nutritional supplements, a vitamin regimen, or a controlled caloric intake.

Orders, treatments, and activities describe the medical instructions that the subject is prescribed to follow based on the designated health condition and the subject's overall state of health. The subject can be prescribed one or more medications. The subject can be prescribed one or more activities, for example, physical exercise, physical therapy, mental aptitude exercises, hygiene techniques, rest, journal-keeping, and health status reporting. The subject can be prescribed one or more nutritional supplements based on the subject's nutritional needs. The subject can be prescribed one or more procedures that can be performed or assisted by a healthcare practitioner or provider. The subject's home can be supplemented with durable medical equipment. The subject can be offered interventions as appropriate.

Monitoring of the subject is accomplished with biometric and telecommunications equipment.

Education and care coordination/integration describes methods of teaching and acclimating the subject to the protocols of the de-centralized healthcare system. Instructive exercises and literature provide training for the subject on, for example, the concept of at-home healthcare, the self-administration of healthcare service, the use of medications, devices and procedures, emergency procedures, information specific to a designated health condition, recordation procedures, and advice on the enjoyment of life in a regulated healthcare environment.

Advanced medical directives describe discussions relating to long-term care or end-of-life care in the at-home environment. The discussions can include, for example, chronic care management and advanced care planning

### Example 2

### A Care Pathway Protocol for Asthma/Bronchitis

A subject is provided healthcare for Asthma/Bronchitis by the protocol of EXAMPLE 1 with the following modifications.

### Designated Health Condition: Asthma/Bronchitis.

### II. Orders, Treatments, and Activities.

A. Medications: Albuterol; Ipratropium; Levalbuterol; Fluticasone and Salmeterol Oral Inhalation; Prednisone; Azithromycin; Ceftriaxone; Phenergan with Codeine; Acetylcysteine; Enoxaparin; Ibuprofen; Alendronate; Nicotine patch; Bupropion; and/or Varenicline.

B. Patient Activities: daily body weight; and/or exercise as tolerated.

C. Nutritional Supplementation: Calcium citrate; and/or Psyllium.

D. Procedures: take menthol/thymol/methyl salicylate/eucalyptol formulation orally; periodic respiratory rate; peak flow measurement before and after bronchodilator treatment; blood pressure and pulse; Saline infusion; Assisted breathing; and/or Smoking cessation.

### Example 3

### A Care Pathway Protocol for Chronic Heart Failure (CHF)

A subject is provided healthcare for CHF by the protocol of EXAMPLE 1 with the following modifications.

### Designated Health Condition: CHF

### II. Orders, Treatments, and Activities.

A. Medications: Furosemide; Torsemide; Zaroxolyn; Hydrochlorothiazide; Enalapril; Lisinopril; Valsortan; Losortan; Asprin; Carvedilol; Metoprolol; Spironolactone; Digoxin; Heparin; Enoxaparin; Isosorbide mononitrate; and/or Hydralazine.

B. Patient Activities: low-impact exercise; and/or daily body weight.

C. Nutritional Supplementation: low-salt diet; and/or fluid restrictions.

D. Procedures: prothrombin time/international normalized ratio (PT/INR) lab; complete blood count; Basic metabolic panel (BMP) and magnesium lab; Brain natriuretic peptide lab; and/or Digoxin level.

### Example 4

### A Care Pathway Protocol for Chronic Obstructive Pulmonary Disease (COPD)

A subject is provided healthcare for COPD by the protocol of EXAMPLE 1 with the following modifications.

### Designated Health Condition: COPD

### II. Orders, Treatments, and Activities.

A. Medications: Albuterol; Ipratropium; Tiotropium; Prednisone; Zithromax; Ceftriaxone; Phenergan with Codeine; and/or Acetylcysteine.

D. Procedures: assisted breathing by oxygen tank/mask.

### Example 5

### A Care Pathway Protocol for Diverticulitis

A subject is provided healthcare for Diverticulitis by the protocol of EXAMPLE 1 with the following modifications.

### Designated Health Condition: Diverticulitis.

### II. Orders, Treatments, and Activities.

A. Medications: Lovenox; and/or Coumadin.

B. Patient Activities: Elevate affected extremity; and/or Measure affected extremity girth daily.

D. Procedures: prothrombin time/international normalized ratio (PT/INR) lab; and/or complete blood count.

### Example 6

### A Care Pathway Protocol for Pneumonia

A subject is provided healthcare for Pneumonia by the protocol of EXAMPLE 1 with the following modifications.

### Designated Health Condition: Pneumonia.

### II. Orders, Treatments, and Activities.

A. Medications: Ceftriaxone; Azithromycin; Moxifloxacin; Phenergan with Codeine; and/or Guaifenesin.

D. Procedures: take menthol/thymol/methyl salicylate/eucalyptol formulation orally.

### Example 7

### Treatment of a Subject with Asthma by a Care Pathway Protocol for Asthma/Bronchitis

A subject candidate having asthma was identified as a candidate for at-home healthcare via review of the candidate's electronic medical records. The candidate was interviewed to determine the status of the asthma episode, the candidate's history of asthma, and the candidate's medical, surgical, and symptom history. The candidate was evaluated for psychological and safety status, and was designated appropriate. The assessment indicated that the candidate was a suitable subject for at-home healthcare, and the candidate was registered as a subject with the de-centralized healthcare system.

A physician investigated the subject's state of health to determine the appropriate orders and nutritional needs. In addition to asthma, the subject was further diagnosed with diabetes mellitus, dyslipidemia, hypertension, and hypokalemia.

The subject's home was inspected for safety and to determine the suitability of the home for adaptation to the device and durable medical equipment requirements of the care pathway protocol. The home was found to be acceptable. The home was equipped with a personal emergency response unit, a two-way audio/video unit, a wireless hub, a weight scale, a blood pressure cuff, and a pulse oximeter.

The subject was prescribed the following medication orders: methylprednisolone sodium succinate (120 mg daily by IV); albuterol (0.083 mg daily by nebulizer); amlodipine (10 mg daily); hydrochlorothiazide/triamterene (25 mg/37.5 mg daily); insulin aspartate (as needed); insulin glargine (12 mg daily); metformin (500 mg daily); montelukast (10 mg daily); potassium chloride (20 mEq daily); simvastatin (20 mg daily); dextromethorphan (liquid formulation as needed); fluticasone propionate/salmeterol (500 mcg/50 mcg by nebulizer as needed); azithromycin/prednisone (30, 20, 10, or 5 mg as needed).

The subject was allowed to follow an ordinary diet and to exercise as tolerated.

The subject spent 3 days in the acute phase, 3 days in the post-acute phase, and 28 days in the episode prevention phase. During this time, the subject was monitored by the central station biometrically and telecommunicatively. The central station made a total of 10 calls to the subject's home to coordinate healthcare procedures and to provide contact with a healthcare practitioner. The dispatch system made one visit to the subject's home to allow a nurse practitioner to make in-person observations of the subject. The subject's blood was periodically analyzed by standard laboratory chemistries to monitor the subject's basic metabolic panel.

The subject did not require food or hygiene services.

After the 34-day period of at-home healthcare, the subject was determined to be in a substantially-improved state of health, and was discharged from the remote-healthcare system. The subject had been fully compliant with the protocol and orders.

### Example 8

### Treatment of a Subject with Chronic Heart Failure (CHF) by a Care Pathway Protocol for Chronic Heart Failure

A subject candidate having CHF was identified as a candidate for at-home healthcare via review of the candidate's electronic medical records. The candidate was interviewed to determine the status of the CHF episode, the candidate's history of CHF, and the candidate's medical, surgical, and symptom history. The candidate was evaluated for psychological and safety status, and was designated appropriate. The assessment indicated that the candidate was a suitable subject for at-home healthcare, and the candidate was registered as a subject with the de-centralized healthcare system.

A physician investigated the subject's state of health to determine the appropriate orders and nutritional needs. In addition to CHF, the subject was further diagnosed with diabetes mellitus, renal insufficiency, osteoarthritis, transient ischemic attack, hyperlipidemia, hypertension, obesity, conjunctivitis, urinary frequency, neuropathy, and hypoxic sleep apnea.

The subject's home was inspected for safety and to determine the suitability of the home for adaptation to the device and durable medical equipment requirements of the care pathway protocol. The home was found to be acceptable. The home was equipped with a personal emergency response unit, a two-way audio/video unit, a wireless hub, a weight scale, a blood pressure cuff, and a pulse oximeter.

The home had been under the care of an external hygiene service prior to inspection. The quality of the hygiene service was found to be acceptable, and further hygiene protocols were not considered.

The subject was prescribed the following medication orders: furosemide (40 mg daily); metolazone (5 mg daily); lisinopril (40 mg daily); carvedilol (3.125 mg daily); clopidogrel (75 mg daily); gabapentin (300 mg daily); glyparamide (2 mg daily); simvastatin (40 mg daily); metozolone (5 mg daily); oxybutynin (5 mg daily); solifenacin (10 mg daily); and amlodipine (10 mg daily).

The subject was ordered to follow a nutritional regimen including potassium (20 mg daily); a low salt-diet; a no-added-sugar/diabetic diet; and a low-fat/low-cholesterol diet. The subject was ordered to undergo an exercise regimen of mobility exercises, pedal cycle exercise, and a progressive exercise program.

The subject spent 8 days in the acute phase, 2 days in the post-acute phase, and 24 days in the episode prevention phase. During this time, the subject was monitored by the central station biometrically and telecommunicatively. The central station made a total of 18 calls to the subject's home to coordinate healthcare procedures and to provide contact with a healthcare practitioner. The dispatch system made a total of 3 visits to the subject's home to allow a nurse practitioner to make in-person observations of the subject. The subject's blood was periodically analyzed by standard laboratory chemistries to monitor the subject's basic metabolic panel and magnesium.

A total of 8 meals were delivered to the subject's home. No hygiene visits were made.

After the 34-day period of at-home healthcare, the subject was determined to be in a substantially-improved state of health, and was discharged from the de-centralized healthcare system. The subject had been fully compliant with the protocol and orders.

### Example 9

### Treatment of a Subject with Chronic Obstructive Pulmonary Disease (COPD) by a Care Pathway Protocol for Chronic Obstructive Pulmonary Disease

A subject candidate having COPD was identified as a candidate for at-home healthcare via review of the candidate's electronic medical records. The candidate was interviewed to determine the status of the COPD episode, the candidate's history of COPD, and the candidate's medical, surgical, and symptom history. The candidate was evaluated for psychological and safety status, and was designated appropriate. The assessment indicated that the candidate was a suitable subject for at-home healthcare, and the candidate was registered as a subject with the de-centralized healthcare system.

A physician investigated the subject's state of health to determine the appropriate orders and nutritional needs. In addition to COPD, the subject was further diagnosed with diabetes mellitus, hyperlipidemia, hypertension, paroxysmal atrial fibrillation, gastroesophageal reflux disease, and h/o pleural effusion.

The subject's home was inspected for safety and to determine the suitability of the home for adaptation to the device and durable medical equipment requirements of the care pathway protocol. The home was found to be acceptable. The home was equipped with a personal emergency response unit, a two-way audio/video unit, a wireless hub, a weight scale, a blood pressure cuff, and a pulse oximeter.

The subject was prescribed the following medication orders: doxycycline (100 mg daily); ipratropium bromide and albuterol sulfate (as needed); albuterol (as needed by nebulizer); prednisone (30, 20, or 10 mg as needed); tiotropium (18 μg as needed by nebulizer); fluticasone propionate/salmeterol (500 mcg/50 mcg by nebulizer as needed); lisinopril (10 mg daily); pravastatin (40 mg daily); metformin (500 mg daily); and promethazine with codeine (liquid formulation as needed).

The subject was ordered to follow a nutritional regimen including a low salt-diet. The subject was ordered to undergo an exercise regimen of pedal cycle exercise 3-times daily, and a progressive exercise program.

The subject spent 4 days in the acute phase, 3 days in the post-acute phase, and 27 days in the episode prevention phase. During this time, the subject was monitored by the central station biometrically and telecommunicatively. The central station made a total of 17 calls to the subject's home to coordinate healthcare procedures and to provide contact with a healthcare practitioner. The dispatch system made one visit to the subject's home to allow a nurse practitioner to make in-person observations of the subject. The subject did not undergo laboratory tests.

The subject did not require food or hygiene services.

After the 34-day period of at-home healthcare, the subject was determined to be in a substantially-improved state of health, and was discharged from the de-centralized healthcare system. The subject had been fully compliant with the protocol and orders.

### Example 10

### Treatment of a Subject with Diverticulitis (DVT) by a Care Pathway Protocol for Diverticulitis

A subject candidate having DVT was identified as a candidate for at-home healthcare via review of the candidate's electronic medical records. The candidate was interviewed to determine the status of the DVT episode, the candidate's history of DVT, and the candidate's medical, surgical, and symptom history. The candidate was evaluated for psychological and safety status, and was designated appropriate. The assessment indicated that the candidate was a suitable subject for at-home healthcare, and the candidate was registered as a subject with the de-centralized healthcare system.

A physician investigated the subject's state of health to determine the appropriate orders and nutritional needs. In addition to DVT, the subject was further diagnosed with dyslipidemia, cellulitis, depression, hypertension, arthritis, obstructive sleep apnea, chronic back pain, and obsesity.

The subject's home was inspected for safety and to determine the suitability of the home for adaptation to the device and durable medical equipment requirements of the care pathway protocol. The home was found to be acceptable. The home was equipped with a personal emergency response unit, a two-way audio/video unit, a wireless hub, a weight scale, a blood pressure cuff, and a pulse oximeter.

The subject was prescribed the following medication orders: enoxaparin (120 mg subcutaneously daily); warfarin (15 mg daily); duloxetine (30 mg daily); clopidogrel (75 mg daily); potassium (20 mEq daily); topiramate (10 mg daily); hydrocodone bitartrate/acetaminophen (10 mg/325 mg daily); tramadol (50 mg daily); clindamycin (150 mg daily); docusate sodium (150 mg daily); lisinopril (40 mg daily); and valsartan (320 mg daily).

The subject was ordered to follow a nutritional regimen including a no sugar added/diabetic diet. The subject was ordered to undergo an exercise regimen of light exercise as tolerated. The subject was educated on the use of coumadin, proper exercise, and smoking cessation techniques.

The subject spent 4 days in the acute phase, 6 days in the post-acute phase, and 24 days in the episode prevention phase. During this time, the subject was monitored by the central station biometrically and telecommunicatively. The central station made a total of 23 calls to the subject's home to coordinate healthcare procedures and to provide contact with a healthcare practitioner. The dispatch system made 2 visits to the subject's home to allow a nurse practitioner to make in-person observations of the subject. The subject's blood was periodically analyzed by standard laboratory chemistries to monitor the subject's prothrombin time/international normalized ratio (PT/INR) and white blood cell count.

The subject did not require food or hygiene services.

After the 34-day period of at-home healthcare, the subject was determined to be in a substantially-improved state of health, and was discharged from the de-centralized healthcare system. The subject had been fully compliant with the protocol and orders.

### Example 11

### Treatment of a Subject with Pneumonia by a Care Pathway Protocol for Pneumonia

A subject candidate having pneumonia was identified as a candidate for at-home healthcare via review of the candidate's electronic medical records. The candidate was interviewed to determine the status of the pneumonia episode, the candidate's history of pneumonia, and the candidate's medical, surgical, and symptom history. The candidate was evaluated for psychological and safety status, and was designated appropriate. The assessment indicated that the candidate was a suitable subject for at-home healthcare, and the candidate was registered as a subject with the de-centralized healthcare system.

A physician investigated the subject's state of health to determine the appropriate orders and nutritional needs. In addition to pneumonia, the subject was further diagnosed with bronchospasm.

The subject's home was inspected for safety and to determine the suitability of the home for adaptation to the device and durable medical equipment requirements of the care pathway protocol. The home was found to be acceptable. The home was equipped with a personal emergency response unit, a two-way audio/video unit, a wireless hub, a weight scale, a blood pressure cuff, and a pulse oximeter.

The subject was prescribed the following medication orders: ceftriaxone (1 g daily by injection); azithromycin (500 mg daily); albuterol 0.083% (2.5 mg as needed by nebulizer); prednisone (10 mg daily); and acetaminophen (325 mg as needed).

The subject was allowed to follow an ordinary diet, and ambulation was encouraged.

The subject spent 3 days in the acute phase, 0 days in the post-acute phase, and 31 days in the episode prevention phase. During this time, the subject was monitored by the central station biometrically and telecommunicatively. The central station made a total of 10 calls to the subject's home to coordinate healthcare procedures and to provide contact with a healthcare practitioner. The dispatch system made 4 visits to the subject's home to allow a nurse practitioner to make in-person observations of the subject. The subject's blood was periodically analyzed by standard laboratory chemistries to monitor the subject's complete blood count with differential.

The subject did not require food or hygiene services.

After the 34-day period of at-home healthcare, the subject was determined to be in a substantially-improved state of health, and was discharged from the de-centralized healthcare system. The subject had been fully compliant with the protocol and orders.

What is claimed is:

1. A de-centralized healthcare system comprising:
a) a central station staffed by at least one healthcare practitioner;
b) a plurality of care pathway protocols, wherein each care pathway protocol comprises a protocol for providing healthcare for a selected designated health condition to a subject in need or want thereof; and
c) a communication system for communication between the subject and the healthcare practitioner,
wherein the subject resides at a location remote from the central station and is registered with the de-centralized healthcare system.

2. The de-centralized healthcare system of claim 1, wherein the central station is effective to monitor the health, status, and location of the subject.

3. The de-centralized healthcare system of claim 1, wherein the central station further comprises a record-keeping system, a computing system, and a library of reference materials, wherein the record-keeping system comprises a record for each subject registered with the de-centralized healthcare system.

4. The de-centralized healthcare system of claim 1, wherein the designated health condition comprises one or more of: heart transplant or implant of heart assist system; craniotomy and endovascular intracranial procedures; extracranial procedures; multiple sclerosis and cerebellar ataxia; intracranial hemorrhage or cerebral infarction; transient ischemia; nonspecific cerebrovascular disorders; seizures; headaches; disequilibrium (vertigo); major chest procedures; other respiratory system operating room procedures; pulmonary embolism; respiratory infections and inflammations; respiratory neoplasms; pulmonary edema and respiratory failure; chronic obstructive pulmonary disease; simple pneumonia and pleurisy; bronchitis and asthma; respiratory signs and symptoms; respiratory system diagnosis with ventilator support; deep venous thrombosis, chronic heart failure; cardiac valve and other major cardiothoracic procedures; cardiac defibrillator implant with cardiac catheterization with acute myocardial infarction/heart failure/shock; cardiac defibrillator implant without cardiac catheterization with major coronary bypass with percutaneous transluminal coronary angioplasty; major cardiovascular procedures or thoracic aorta; permanent cardiac pacemaker implant; percutaneous cardiovascular procedure with drug-eluting stent or other circulatory system operating room procedures; other vascular procedures; cardiac pacemaker revision except device replacement; other circulatory system operating room procedures; acute myocardial infarction; circulatory disorders except acute myocardial infarction, with cardiac catheterization; heart failure and shock; deep vein thrombophlebitis; dehydration; diversion diagnosis; end-stage disease states; external feeding; New York Heart Association class IV heart failure; severe/symptomatic valvular heart disease in individuals who are poor surgical candidates; Global Initiative for Chronic Obstructive Lung Disease classification stage IV COPD/respiratory failure; malignant disease; failure to thrive of senescence; acquired immunodeficiency syndrome (AIDS); cirrhosis in individuals who are not candidates for transplantation; amyotrophic lateral sclerosis; peripheral vascular disorders; atherosclerosis; hypertension; cardiac arrhythmia and conduction disorders; syncope and collapse; chest pain; other circulatory system diagnoses; diverticulitis; post-operative central nervous system procedures; strokes; neoplasms and lymphoma-related hospitalizations; chronic infections; stomach, esophageal and duodenal procedures; major small and large bowel procedures with other digestive system operating room procedures; peritoneal adhesiolysis; hernia procedures except inguinal and femoral; other digestive system operating room procedures; major gastrointestinal disorders and peritoneal infection; digestive malignancy; gastrointestinal hemorrhage; complicated peptic ulcer; gastrointestinal obstruction; esophagitis, gastroenterological and miscellaneous digestive disorders; other digestive system diagnoses; laparoscopic cholecystectomy without common bile duct exploration; cirrhosis and alcoholic hepatitis; malignancy of hepatobiliary system or pancreas; disorders of pancreas except malignancy; disorders of liver except malignancy, cirrhosis, alcoholic hepatitis; disorders of the biliary tract; spinal fusion except cervical; wound debridement and skin graft except hand, for musculoskeletal-connective tissue; revision of hip or knee replacement; major joint replacement or reattachment of lower extremity; hip and femur procedures except major joint; knee procedures with past diagnosis of infection; back and neck procedures except spinal fusion or disctectomy; lower extremety and humer procedures except hip, foot, femur; other musculoskeletal system and connective tissue operating room procedures; fractures of hip and pelvis; osteomyelitis; pathological fractures and musculoskeletal and connective tissue fractures, sprains, strains and dislocations except femur, hip, pelvis and skin graft and/or debridement for skin ulceration or cellulitis; connective tissue disorders; medical back problems; tendonitis, myositis and bursitis; fracture, sprain, strain and dislocation except femur, hip, and pelvis; skin graft and/or debridement for skin ulcer or cellulitis; other skin, subcutaneous tissue and breast procedures; skin ulcers; cellulitis; skin grafts and wound debridement for endocrine, nutrition, and metabolic disorders; other endocrine, nutrition and metabolic operating room procedures; diabetes; nutritional and miscellaneous metabolic disorders; endocrine disorders; major bladder procedures; kidney and ureter procedures for neoplasm; transurethral procedures; other kidney and urinary tract procedures; renal failure; kidney and urinary tract infections; urinary stones without electronic shockwave lithotripsy; other kidney and urinary tract diagnoses; major male pelvic procedures, conization, laparoscopy and tubal interruption; other female reproductive system operating procedures; lymphoma and non-acute leukemia; infectious and parasitic diseases with operating room procedure; postoperative or post-traumatic infections within operating rooms; fever; viral illness; septicemia or severe sepsis without mechanical ventilation for at least 96 hours; poisoning and toxic effects of drugs; complications of treatment; signs and symptoms; HIV with major related conditions; extensive operating room procedure unrelated to principal disorder; and non-extensive operating room procedure unrelated to principal disorder.

5. The de-centralized healthcare system of claim 1, wherein the de-centralized healthcare system affiliates, collaborates, communicates, or cooperates with at least one conventional healthcare facility.

6. The de-centralized healthcare system of claim 1, wherein each care pathway protocol comprises:
a) subject qualification and admission, comprising:
1) stability criteria, comprising: subject status, psychological and social safety status; and
2) assessments, comprising: subject's health, home assessments, and nutritional needs;
b) orders, treatments, and activities, comprising: medications, subject activities, nutritional supplementation, procedures, durable medical equipment, and optionally interventions effective to improve the health of the subject;
c) monitoring, comprising: biometric and telecommunication equipment to monitor the subject;
d) education and care coordination/integration, comprising: instructive exercises and literature; and
e) advanced medical directive, comprising: discussions regarding one or both of chronic care management and advanced care planning.

7. The de-centralized healthcare system of claim 1, wherein the central station is staffed by at least two healthcare practitioners.

8. The de-centralized healthcare system of claim 1, wherein the healthcare practitioner is a doctor of medicine, a doctor of osteopathy, or a nurse practitioner.

9. The de-centralized healthcare system of claim 1, wherein the healthcare practitioner is available by appointment, on-call, or for emergencies.

10. The de-centralized healthcare system of claim 1, wherein the healthcare practitioner provides a healthcare service to the subject.

11. The de-centralized healthcare system of claim 1, wherein the subject comprises a designated health condition.

12. The de-centralized healthcare system of claim 1, wherein the healthcare practitioner is a veterinarian.

13. The de-centralized healthcare system of claim 12, wherein the subject is the owner of an animal.

14. The de-centralized healthcare system of claim 13, wherein the animal is in need of healthcare or the owner is in of want of healthcare for the animal.

15. The de-centralized healthcare system of claim 14, wherein the animal is a pet, a guardian, a livestock, a service animal, a show animal, a working animal, a racing animal, or a breeding animal.

16. The de-centralized healthcare system of claim 14, wherein the animal is a mammal, a reptile, a bird, an amphibian, or an aquatic animal.

17. The de-centralized healthcare system of claim 1, wherein the subject resides at a private home, a nursing home, an apartment, a hotel, or an assisted-living facility.

18. The de-centralized healthcare system of claim 1, wherein the subject does not leave home to receive healthcare service.

19. The de-centralized healthcare system of claim 1, wherein the subject requests a healthcare service via the communication system.

20. The de-centralized healthcare system of claim 19, wherein the healthcare service comprises an advice, an instruction, or a recommendation.

21. The de-centralized healthcare system of claim 1, wherein the subject is educated in self-administration of a healthcare procedure, wherein the healthcare procedure comprises a self-injection of an injectable material, a self-medication with a medication, a self-application of a topical material, a self-examination, a use of a device, a physical therapy, or a collection of a bodily fluid, a tissue or a discharge.

22. The de-centralized healthcare system of claim 21, wherein the healthcare procedure comprises the use of a kit provided by the de-centralized healthcare system.

23. The de-centralized healthcare system of claim 1, wherein the subject is at or near the end-of-life.

24. The de-centralized healthcare system of claim 1, wherein each care pathway protocol of the plurality of care pathway protocols is effective to provide healthcare to the subject for one or more designated health conditions.

25. The de-centralized healthcare system of claim 24, wherein each designated health condition has at least one care pathway protocol.

26. The de-centralized healthcare system of claim 24, wherein each of the care pathway protocols is used independently.

27. The de-centralized healthcare system of claim 1, wherein the healthcare practitioner performs at least one of the care pathway protocols.

28. The de-centralized healthcare system of claim 1, wherein more than one care pathway protocols are used synergistically.

29. The de-centralized healthcare system of claim 1, wherein the healthcare practitioner diagnoses, evaluates, or assesses the subject via the communication system.

30. The de-centralized healthcare system of claim 1, wherein the communication system comprises one or more of written, audio, visual, monitoring, and audio/visual communication.

31. The de-centralized healthcare system of claim 1, wherein the healthcare practitioner provides a healthcare service to the subject via the communication system.

32. The de-centralized healthcare system of claim 31, wherein the healthcare service comprises one or more of an advice, an instruction, a recommendation, an inquiry, or a prescription.

33. The de-centralized healthcare system of claim 1, further comprising a dispatch system.

34. The de-centralized healthcare system of claim 33, wherein the dispatch system is located at the same facility as the central station.

35. The de-centralized healthcare system of claim 33, wherein the dispatch system is located at a different facility than the central station.

36. The de-centralized healthcare system of claim 33, wherein the dispatch system is staffed by at least one healthcare provider.

37. The de-centralized healthcare system of claim 36, wherein the healthcare provider is a physician, a clinician, a nurse, a paramedic, a therapist, a pharmacist, or a veterinarian.

38. The de-centralized healthcare system of claim 37, wherein the physician is a doctor of medicine, osteopathy, chiropractic, podiatry, optometry, or dentistry.

39. The de-centralized healthcare system of claim 36, wherein the dispatch system dispatches the healthcare provider to the subject, wherein the subject is in need or want of healthcare.

40. The de-centralized healthcare system of claim 39, wherein the healthcare provider provides a healthcare service to the subject according to a care pathway protocol.

41. The de-centralized healthcare system of claim 39, wherein the subject requests that the dispatch system dispatch the healthcare provider to the subject.

42. The de-centralized healthcare system of claim 41, wherein the subject requests an emergency healthcare service.

43. The de-centralized healthcare system of claim 39, wherein the subject had not previously requested that the dispatch system dispatch the healthcare provider to the subject.

44. The de-centralized healthcare system of claim 43, wherein a monitoring device alerts the central station to a need for the emergency healthcare service.

45. The de-centralized healthcare system of claim 33, further comprising a plurality of kits, wherein each kit independently comprises materials effective for self-administration of a healthcare service.

46. The de-centralized healthcare system of claim 45, further comprising a laboratory.

47. The de-centralized healthcare system of claim 46, wherein the laboratory comprises facilities for performing an analysis of a sample provided by the subject.

48. The de-centralized healthcare system of claim 46, further comprising a hygiene system.

49. The de-centralized healthcare system of claim 48, wherein the hygiene system is staffed by at least one hygienist, wherein the hygienist enters the residence of the subject to perform one or more hygiene duties.

50. The de-centralized healthcare system of claim 49, wherein the subject is incapable of performing the hygiene duties.

51. The de-centralized healthcare system of claim 49, wherein the hygienist enters the residence of the subject daily, semi-weekly, weekly, biweekly, or monthly.

52. The de-centralized healthcare system of claim 49, wherein the hygiene duties comprise laundry, cleaning bed sheets, sanitizing, and disinfecting.

53. The de-centralized healthcare system of claim 48, further comprising a food delivery system.

54. The de-centralized healthcare system of claim 53, wherein the subject is incapable of providing food for the subject.

55. The de-centralized healthcare system of claim 53, wherein the food delivery system provides daily, semi-weekly, weekly, bi-weekly, or monthly delivery of a food supply.

56. The de-centralized healthcare system of claim 55, wherein the food supply comprises one or more of prepared meals, packaged foods, frozen foods, groceries, nutritional supplements, and foods comprising a nutritional regimen.

57. A method of providing healthcare, the method comprising:
a) providing a central station for the management of a de-centralized healthcare system, wherein the central station is staffed by at least one healthcare practitioner;
b) registering a subject in the de-centralized healthcare system, wherein the subject comprises a designated health condition;
c) providing the subject with means for a self-administered healthcare procedure; and
d) providing a communication system comprising communication between the subject and the central station, whereby the healthcare practitioner advises the subject on performing the self-administered healthcare procedure according to a care pathway protocol,
wherein the subject resides at a location remote from the central station.

58. The method of claim 57, wherein the designated health condition comprises one or more of: heart transplant or implant of heart assist system; craniotomy and endovascular intracranial procedures; extracranial procedures; multiple sclerosis and cerebellar ataxia; intracranial hemorrhage or cerebral infarction; transient ischemia; nonspecific cerebrovascular disorders; seizures; headaches; disequilibrium (vertigo); major chest procedures; other respiratory system operating room procedures; pulmonary embolism; respiratory infections and inflammations; respiratory neoplasms; pulmonary edema and respiratory failure; chronic obstructive pulmonary disease; simple pneumonia and pleurisy; bronchitis and asthma; respiratory signs and symptoms; respiratory system diagnosis with ventilator support; deep venous thrombosis, chronic heart failure; cardiac valve and other major cardiothoracic procedures; cardiac defibrillator implant with cardiac catheterization with acute myocardial infarction/heart failure/shock; cardiac defibrillator implant without cardiac catheterization with major coronary bypass with percutaneous transluminal coronary angioplasty; major cardiovascular procedures or thoracic aorta; permanent cardiac pacemaker implant; percutaneous cardiovascular procedure with drug-eluting stent or other circulatory system operating room procedures; other vascular procedures; cardiac pacemaker revision except device replacement; other circulatory system operating room procedures; acute myocardial infarction; circulatory disorders except acute myocardial infarction, with cardiac catheterization; heart failure and shock; deep vein thrombophlebitis; dehydration; diversion diagnosis; end-stage disease states; external feeding; New York Heart Association class IV heart failure; severe/symptomatic valvular heart disease in individuals who are poor surgical candidates; Global Initiative for Chronic Obstructive Lung Disease classification stage IV COPD/respiratory failure; malignant disease; failure to thrive of senescence; acquired immunodeficiency syndrome (AIDS); cirrhosis in individuals who are not candidates for transplantation; amyotrophic lateral sclerosis; peripheral vascular disorders; atherosclerosis; hypertension; cardiac arrhythmia and conduction disorders; syncope and collapse; chest pain; other circulatory system diagnoses; diverticulitis; post-operative central nervous system procedures; strokes; neoplasms and lymphoma-related hospitalizations; chronic infections; stomach, esophageal and duodenal procedures; major small and large bowel procedures with other digestive system operating room procedures; peritoneal adhesiolysis; hernia procedures except inguinal and femoral; other digestive system operating room procedures; major gastrointestinal disorders and peritoneal infection; digestive malignancy; gastrointestinal hemorrhage; complicated peptic ulcer; gastrointestinal obstruction; esophagitis, gastroenterological and miscellaneous digestive disorders; other digestive system diagnoses; laparoscopic cholecystectomy without common bile duct exploration; cirrhosis and alcoholic hepatitis; malignancy of hepatobiliary system or pancreas; disorders of pancreas except malignancy; disorders of liver except malignancy, cirrhosis, alcoholic hepatitis; disorders of the biliary tract; spinal fusion except cervical; wound debridement and skin graft except hand, for musculoskeletal-connective tissue; revision of hip or knee replacement; major joint replacement or reattachment of lower extremity; hip and femur procedures except major joint; knee procedures with past diagnosis of infection; back and neck procedures except spinal fusion or disctectomy; lower extremety and humer procedures except hip, foot, femur; other musculoskeletal system and connective tissue operating room procedures; fractures of hip and pelvis; osteomyelitis; pathological fractures and musculoskeletal and connective tissue fractures, sprains, strains and dislocations except femur, hip, pelvis and skin graft and/or debridement for skin ulceration or cellulitis; connective tissue disorders; medical back problems; tendonitis, myositis and bursitis; fracture, sprain, strain and dislocation except femur, hip, and pelvis; skin graft and/or debridement for skin ulcer or cellulitis; other skin, subcutaneous tissue and breast procedures; skin ulcers; cellulitis; skin grafts and wound debridement for endocrine, nutrition, and metabolic disorders; other endocrine, nutrition and metabolic operating room procedures; diabetes; nutritional and miscellaneous metabolic disorders; endocrine disorders; major bladder procedures; kidney and ureter procedures for neoplasm; transurethral procedures; other kidney and urinary tract procedures; renal failure; kidney and urinary tract infections; urinary stones without electronic shockwave lithotripsy; other kidney and urinary tract diagnoses; major male pelvic procedures, conization, laparoscopy and tubal interruption; other female reproductive system operating procedures; lymphoma and non-acute leukemia; infectious and parasitic diseases with operating room procedure; postoperative or post-traumatic infections within operating rooms; fever; viral illness; septicemia or severe sepsis without mechanical ventilation for at least 96 hours; poisoning and toxic effects of drugs; complications of treatment; signs and symptoms; HIV with major related conditions; extensive operating room procedure unrelated to principal disorder; and non-extensive operating room procedure unrelated to principal disorder.

59. The method of claim 57, wherein the subject resides at a private home, a nursing home, an apartment, a hotel, or an assisted-living facility.

60. The method of claim 57, wherein the self-administered healthcare procedure comprises one or more of a medical regimen, an exercise regimen, a nutritional regimen, a mental aptitude regimen, a morale regimen, a hygiene regimen, a self-injection of an injectable material, a self-medication with a medication, a self-application of a topical material, a self-examination, a use of a device, a physical therapy, or a collection of a bodily fluid, a tissue or a discharge.

61. The method of claim 57, wherein the communication system comprises written, audio, visual, monitoring, or audio/visual communication, or a combination thereof.

62. The method of claim 57, wherein the healthcare practitioner diagnoses, evaluates, or assesses the subject via the communication system.

63. The method of claim 57, wherein the care pathway protocol comprises:
a) subject qualification and admission, comprising:
1) stability criteria, comprising: subject status, psychological and social safety status; and
2) assessments, comprising: subject's health, home assessments, and nutritional needs;
b) orders, treatments, and activities, comprising: medications, subject activities, nutritional supplementation, procedures, durable medical equipment, and optionally interventions effective to improve the health of the subject;
c) monitoring, comprising: biometric and telecommunication equipment to monitor the subject;
d) education and care coordination/integration, comprising: instructive exercises and literature; and
e) advanced medical directive, comprising: discussions regarding one or both of chronic care management and advanced care planning.

64. The method of claim 57, further comprising providing at-home healthcare to the subject wherein the subject is in need or want of at-home healthcare.

65. The method of claim 64, wherein the subject requests at-home healthcare.

66. The method of claim 64, wherein a device alerts the central station to a need for at-home healthcare.

67. The method of claim 57, further comprising:
a) providing a dispatch system staffed by at least one healthcare provider;
b) dispatching the healthcare provider from the dispatch system to the subject, wherein the subject is in need or want of healthcare; and
c) administering a healthcare service to the subject via the healthcare provider according to a care pathway protocol.

68. The method of claim 67, wherein the healthcare provider is a physician, a clinician, a nurse, a paramedic, a therapist, a pharmacist, or a veterinarian.

69. The method of claim 68, wherein the physician is a doctor of medicine, osteopathy, chiropractic, podiatry, optometry, or dentistry.

70. A method of providing at-home healthcare, the method comprising:
a) providing a central station for the management of a de-centralized healthcare system;
b) providing a dispatch system staffed by at least one healthcare provider;
c) registering a subject in the de-centralized healthcare system, wherein the subject comprises a designated health condition;
d) providing a communication system comprising audio/visual communication between the subject and the central station, optionally whereby the subject requests healthcare;
e) optionally providing one or more devices and durable medical equipment to the home of the subject, wherein the devices are effective to alert the central station to a need for at-home healthcare;
f) dispatching the healthcare provider from the dispatch system to the subject, wherein the subject is in need or want of healthcare; and
g) administering a healthcare service to the subject via the healthcare provider according to a care pathway protocol.

71. A method of selecting a subject for a de-centralized healthcare system, the method comprising:
a) providing a subject comprising a condition;
b) providing a list comprising a plurality of designated health conditions;
c) diagnosing the condition of the subject to provide a diagnosed condition;
d) comparing the diagnosed condition to the list comprising a plurality of designated health conditions;
e) selecting a subject based on the results of step d) to provide a selected subject; and
f) registering the selected subject in the de-centralized healthcare system to provide a registered subject,
wherein the de-centralized healthcare system comprises:
1) a central station staffed by at least one healthcare practitioner;
2) a plurality of care pathway protocols, wherein each care pathway protocol comprises a protocol for providing healthcare for a selected designated health condition; and
3) a communication system for communication between the registered subject and the healthcare practitioner,
wherein the registered subject resides at a location remote from the central station.

72. The method of claim 71, wherein the designated health condition comprises one or more of: heart transplant or implant of heart assist system; craniotomy and endovascular intracranial procedures; extracranial procedures; multiple sclerosis and cerebellar ataxia; intracranial hemorrhage or cerebral infarction; transient ischemia; nonspecific cerebrovascular disorders; seizures; headaches; disequilibrium (vertigo); major chest procedures; other respiratory system operating room procedures; pulmonary embolism; respiratory infections and inflammations; respiratory neoplasms; pulmonary edema and respiratory failure; chronic obstructive pulmonary disease; simple pneumonia and pleurisy; bronchitis and asthma; respiratory signs and symptoms; respiratory system diagnosis with ventilator support; deep venous thrombosis, chronic heart failure; cardiac valve and other major cardiothoracic procedures; cardiac defibrillator implant with cardiac catheterization with acute myocardial infarction/heart failure/shock; cardiac defibrillator implant without cardiac catheterization with major coronary bypass with percutaneous transluminal coronary angioplasty; major cardiovascular procedures or thoracic aorta; permanent cardiac pacemaker implant; percutaneous cardiovascular procedure with drug-eluting stent or other circulatory system operating room procedures; other vascular procedures; cardiac pacemaker revision except device replacement; other circulatory system operating room procedures; acute myocardial infarction; circulatory disorders except acute myocardial infarction, with cardiac catheterization; heart failure and shock; deep vein thrombophlebitis; dehydration; diversion diagnosis; end-stage disease states; external feeding; New York Heart Association class IV heart failure; severe/symptomatic valvular heart disease in individuals who are poor surgical candidates; Global Initiative for Chronic Obstructive Lung Disease classification stage IV COPD/respiratory failure; malignant disease; failure to thrive of senescence; acquired immunodeficiency syndrome (AIDS); cirrhosis in individuals who are not candidates for transplantation; amyotrophic lateral sclerosis; peripheral vascular disorders; atherosclerosis; hypertension; cardiac arrhythmia and conduction disorders; syncope and collapse; chest pain; other circulatory system diagnoses; diverticulitis; post-operative central nervous system procedures; strokes; neoplasms and lymphoma-related hospitalizations; chronic infections; stomach, esophageal and duodenal procedures; major small and large bowel procedures with other digestive system operating room procedures; peritoneal adhesiolysis; hernia procedures except inguinal and femoral; other digestive system operating room procedures; major gastrointestinal disorders and peritoneal infection; digestive malignancy; gastrointestinal hemorrhage; complicated peptic ulcer; gastrointestinal obstruction; esophagitis, gastroenterological and miscellaneous digestive disorders; other digestive system diagnoses; laparoscopic cholecystectomy without common bile duct exploration; cirrhosis and alcoholic hepatitis; malignancy of hepatobiliary system or pancreas; disorders of pancreas except malignancy; disorders of liver except malignancy, cirrhosis, alcoholic hepatitis; disorders of the biliary tract; spinal fusion except cervical; wound debridement and skin graft except hand, for musculoskeletal-connective tissue; revision of hip or knee replacement; major joint replacement or reattachment of lower extremity; hip and femur procedures except major joint; knee procedures with past diagnosis of infection; back and neck procedures except spinal fusion or disctectomy; lower extremety and humer procedures except hip, foot, femur; other musculoskeletal system and connective tissue operating room procedures; fractures of hip and pelvis; osteomyelitis; pathological fractures and musculoskeletal and connective tissue fractures, sprains, strains and dislocations except femur, hip, pelvis and skin graft and/or debridement for skin ulceration or cellulitis; connective tissue disorders; medical back problems; tendonitis, myositis and bursitis; fracture, sprain, strain and dislocation except femur, hip, and pelvis; skin graft and/or debridement for skin ulcer or cellulitis; other skin, subcutaneous tissue and breast procedures; skin ulcers; cellulitis; skin grafts and wound debridement for endocrine, nutrition, and metabolic disorders; other endocrine, nutrition and metabolic operating room procedures; diabetes; nutritional and miscellaneous metabolic disorders; endocrine disorders; major bladder procedures; kidney and ureter procedures for neoplasm; transurethral procedures; other kidney and urinary tract procedures; renal failure; kidney and urinary tract infections; urinary stones without electronic shockwave lithotripsy; other kidney and urinary tract diagnoses; major male pelvic procedures, conization, laparoscopy and tubal interruption; other female reproductive system operating procedures; lymphoma and non-acute leukemia; infectious and parasitic diseases with operating room procedure; postoperative or post-traumatic infections within operating rooms; fever; viral illness; septicemia or severe sepsis without mechanical ventilation for at least 96 hours; poisoning and toxic effects of drugs; complications of treatment; signs and symptoms; HIV with major related conditions; extensive operating room procedure unrelated to principal disorder; and non-extensive operating room procedure unrelated to principal disorder.

73. A method of selecting a subject for a de-centralized healthcare system, the method comprising:
a) providing a subject comprising a condition;
b) diagnosing the condition of the subject to provide a diagnosed condition;
c) performing a cost/benefit analysis for caring for the subject in a hospital system;
d) performing a cost/benefit analysis for caring for the subject in the de-centralized healthcare system;
e) comparing the results of steps c) and d);
f) selecting a subject based on the results of step e) to provide a selected subject; and
g) registering the selected subject in the de-centralized healthcare system to provide a registered subject,
wherein the de-centralized healthcare system comprises:
1) a central station staffed by at least one healthcare practitioner;
2) a plurality of care pathway protocols, wherein each care pathway protocol comprises a protocol for providing healthcare for a selected designated health condition; and
3) a communication system for communication between the registered subject and the healthcare practitioner,
wherein the registered subject resides at a location remote from the central station.

74. A care pathway protocol, comprising:
a) subject qualification and admission, comprising:
1) stability criteria, comprising: subject status, psychological and social safety status; and
2) assessments, comprising: subject's health, home assessments, and nutritional needs;
b) orders, treatments, and activities, comprising: medications, subject activities, nutritional supplementation, procedures, durable medical equipment, and optionally interventions effective to improve the health of the subject;
c) monitoring, comprising: biometric and telecommunication equipment to monitor the subject;
d) education and care coordination/integration, comprising: instructive exercises and literature; and
e) advanced medical directive, comprising: discussions regarding one or both of chronic care management and advanced care planning.

75. The care pathway protocol of claim 74, wherein the care pathway protocol is effective to provide therapy for one or more of: heart transplant or implant of heart assist system; craniotomy and endovascular intracranial procedures; extracranial procedures; multiple sclerosis and cerebellar ataxia; intracranial hemorrhage or cerebral infarction; transient ischemia; nonspecific cerebrovascular disorders; seizures; headaches; disequilibrium (vertigo); major chest procedures; other respiratory system operating room procedures; pulmonary embolism; respiratory infections and inflammations; respiratory neoplasms; pulmonary edema and respiratory failure; chronic obstructive pulmonary disease; simple pneumonia and pleurisy; bronchitis and asthma; respiratory signs and symptoms; respiratory system diagnosis with ventilator support; deep venous thrombosis, chronic heart failure; cardiac valve and other major cardiothoracic procedures; cardiac defibrillator implant with cardiac catheterization with acute myocardial infarction/heart failure/shock; cardiac defibrillator implant without cardiac catheterization with major coronary bypass with percutaneous transluminal coronary angioplasty; major cardiovascular procedures or thoracic aorta; permanent cardiac pacemaker implant; percutaneous cardiovascular procedure with drug-eluting stent or other circulatory system operating room procedures; other vascular procedures; cardiac pacemaker revision except device replacement; other circulatory system operating room procedures; acute myocardial infarction; circulatory disorders except acute myocardial infarction, with cardiac catheterization; heart failure and shock; deep vein thrombophlebitis; dehydration; diversion diagnosis; end-stage disease states; external feeding; New York Heart Association class IV heart failure; severe/symptomatic valvular heart disease in individuals who are poor surgical candidates; Global Initiative for Chronic Obstructive Lung Disease classification stage IV COPD/respiratory failure; malignant disease; failure to thrive of senescence; acquired immunodeficiency syndrome (AIDS); cirrhosis in individuals who are not candidates for transplantation; amyotrophic lateral sclerosis; peripheral vascular disorders; atherosclerosis; hypertension; cardiac arrhythmia and conduction disorders; syncope and collapse; chest pain; other circulatory system diagnoses; diverticulitis; post-operative central nervous system procedures; strokes; neoplasms and lymphoma-related hospitalizations; chronic infections; stomach, esophageal and duodenal procedures; major small and large bowel procedures with other digestive system operating room procedures; peritoneal adhesiolysis; hernia procedures except inguinal and femoral; other digestive system operating room procedures; major gastrointestinal disorders and peritoneal infection; digestive malignancy; gastrointestinal hemorrhage; complicated peptic ulcer; gastrointestinal obstruction; esophagitis, gastroenterological and miscellaneous digestive disorders; other digestive system diagnoses; laparoscopic cholecystectomy without common bile duct exploration; cirrhosis and alcoholic hepatitis; malignancy of hepatobiliary system or pancreas; disorders of pancreas except malignancy; disorders of liver except malignancy, cirrhosis, alcoholic hepatitis; disorders of the biliary tract; spinal fusion except cervical; wound debridement and skin graft except hand, for musculoskeletal-connective tissue; revision of hip or knee replacement; major joint replacement or reattachment of lower extremity; hip and femur procedures except major joint; knee procedures with past diagnosis of infection; back and neck procedures except spinal fusion or disctectomy; lower extremety and humer procedures except hip, foot, femur; other musculoskeletal system and connective tissue operating room procedures; fractures of hip and pelvis; osteomyelitis; pathological fractures and musculoskeletal and connective tissue fractures, sprains, strains and dislocations except femur, hip, pelvis and skin graft and/or debridement for skin ulceration or cellulitis; connective tissue disorders; medical back problems; tendonitis, myositis and bursitis; fracture, sprain, strain and dislocation except femur, hip, and pelvis; skin graft and/or debridement for skin ulcer or cellulitis; other skin, subcutaneous tissue and breast procedures; skin ulcers; cellulitis; skin grafts and wound debridement for endocrine, nutrition, and metabolic disorders; other endocrine, nutrition and metabolic operating room procedures; diabetes; nutritional and miscellaneous metabolic disorders; endocrine disorders; major bladder procedures; kidney and ureter procedures for neoplasm; transurethral procedures; other kidney and urinary tract procedures; renal failure; kidney and urinary tract infections; urinary stones without electronic shockwave lithotripsy; other kidney and urinary tract diagnoses; major male pelvic procedures, conization, laparoscopy and tubal interruption; other female reproductive system operating procedures; lymphoma and non-acute leukemia; infectious and parasitic diseases with operating room procedure; postoperative or post-traumatic infections within operating rooms; fever; viral illness; septicemia or severe sepsis without mechanical ventilation for at least 96 hours; poisoning and toxic effects of drugs; complications of treatment; signs and symptoms; HIV with major related conditions; extensive operating room procedure unrelated to principal disorder; and non-extensive operating room procedure unrelated to principal disorder.

76. A method of providing acute healthcare, the method comprising:
a) qualifying and admitting a subject, comprising:
1) evaluating the subject's stability criteria, comprising the subject's status, and psychological and social safety status; and
2) assessing the subject's health, home, and nutritional needs;
b) issuing orders, treatments, and activities for the subject, comprising: medications, subject activities, nutritional supplementation, procedures, durable medical equipment, and optionally interventions effective to improve the health of the subject;
c) monitoring the subject, comprising: providing biometric and telecommunication equipment to monitor the subject and monitoring the subject with the biometric and telecommunication equipment;
d) educating the subject on care coordination/integration, the educating comprising: instructive exercises and literature; and
e) providing an advanced medical directive, comprising: discussing one or both of chronic care management and advanced care planning.

77. The method of claim 76, wherein the subject is in need or want of therapy for one or more of: heart transplant or implant of heart assist system; craniotomy and endovascular intracranial procedures; extracranial procedures; multiple sclerosis and cerebellar ataxia; intracranial hemorrhage or cerebral infarction; transient ischemia; nonspecific cerebrovascular disorders; seizures; headaches; disequilibrium (vertigo); major chest procedures; other respiratory system operating room procedures; pulmonary embolism; respiratory infections and inflammations; respiratory neoplasms; pulmonary edema and respiratory failure; chronic obstructive pulmonary disease; simple pneumonia and pleurisy; bronchitis and asthma; respiratory signs and symptoms; respiratory system diagnosis with ventilator support; deep venous thrombosis, chronic heart failure; cardiac valve and other major cardiothoracic procedures; cardiac defibrillator implant with cardiac catheterization with acute myocardial infarction/heart failure/shock; cardiac defibrillator implant without cardiac catheterization with major coronary bypass with percutaneous transluminal coronary angioplasty; major cardiovascular procedures or thoracic aorta; permanent cardiac pacemaker implant; percutaneous cardiovascular procedure with drug-eluting stent or other circulatory system operating room procedures; other vascular procedures; cardiac pacemaker revision except device replacement; other circulatory system operating room procedures; acute myocardial infarction; circulatory disorders except acute myocardial infarction, with cardiac catheterization; heart failure and shock; deep vein thrombophlebitis; dehydration; diversion diagnosis; end-stage disease states; external feeding; New York Heart Association class IV heart failure; severe/symptomatic valvular heart disease in individuals who are poor surgical candidates; Global Initiative for Chronic Obstructive Lung Disease classification stage IV COPD/respiratory failure; malignant disease; failure to thrive of senescence; acquired immunodeficiency syndrome (AIDS); cirrhosis in individuals who are not candidates for transplantation; amyotrophic lateral sclerosis; peripheral vascular disorders; atherosclerosis; hypertension; cardiac arrhythmia and conduction disorders; syncope and collapse; chest pain; other circulatory system diagnoses; diverticulitis; post-operative central nervous system procedures; strokes; neoplasms and lymphoma-related hospitalizations; chronic infections; stomach, esophageal and duodenal procedures; major small and large bowel procedures with other digestive system operating room procedures; peritoneal adhesiolysis; hernia procedures except inguinal and femoral; other digestive system operating room procedures; major gastrointestinal disorders and peritoneal infection; digestive malignancy; gastrointestinal hemorrhage; complicated peptic ulcer; gastrointestinal obstruction; esophagitis, gastroenterological and miscellaneous digestive disorders; other digestive system diagnoses; laparoscopic cholecystectomy without common bile duct exploration; cirrhosis and alcoholic hepatitis; malignancy of hepatobiliary system or pancreas; disorders of pancreas except malignancy; disorders of liver except malignancy, cirrhosis, alcoholic hepatitis; disorders of the biliary tract; spinal fusion except cervical; wound debridement and skin graft except hand, for musculoskeletal-connective tissue; revision of hip or knee replacement; major joint replacement or reattachment of lower extremity; hip and femur procedures except major joint; knee procedures with past diagnosis of infection; back and neck procedures except spinal fusion or disctectomy; lower extremety and humer procedures except hip, foot, femur; other musculoskeletal system and connective tissue operating room procedures; fractures of hip and pelvis; osteomyelitis; pathological fractures and musculoskeletal and connective tissue fractures, sprains, strains and dislocations except femur, hip, pelvis and skin graft and/or debridement for skin ulceration or cellulitis; connective tissue disorders; medical back problems; tendonitis, myositis and bursitis; fracture, sprain, strain and dislocation except femur, hip, and pelvis; skin graft and/or debridement for skin ulcer or cellulitis; other skin, subcutaneous tissue and breast procedures; skin ulcers; cellulitis; skin grafts and wound debridement for endocrine, nutrition, and metabolic disorders; other endocrine, nutrition and metabolic operating room procedures; diabetes; nutritional and miscellaneous metabolic disorders; endocrine disorders; major bladder procedures; kidney and ureter procedures for neoplasm; transurethral procedures; other kidney and urinary tract procedures; renal failure; kidney and urinary tract infections; urinary stones without electronic shockwave lithotripsy; other kidney and urinary tract diagnoses; major male pelvic procedures, conization, laparoscopy and tubal interruption; other female reproductive system operating procedures; lymphoma and non-acute leukemia; infectious and parasitic diseases with operating room procedure; postoperative or post-traumatic infections within operating rooms; fever; viral illness; septicemia or severe sepsis without mechanical ventilation for at least 96 hours; poisoning and toxic effects of drugs; complications of treatment; signs and symptoms; HIV with major related conditions; extensive operating room procedure unrelated to principal disorder; and non-extensive operating room procedure unrelated to principal disorder.
